SEQUENCE LISTING (212 sequences)

SEQ ID NO: 1 - protein, 127 aa, synthetic construct
  REGION 1..127: note = synthetic
EVKLVESGGGLVQPGGSLSLSCAASGFAFTDYYMNWVRQPPGKALEWLALIRNKAAGYTIYYSASVKGRFTISRDNSQSILYLQMTALRPEDSATYYCVRDMTIYDGYYVDYALDYWGQGTSVTVSS

SEQ ID NO: 2 - protein, 107 aa, synthetic construct
  REGION 1..107: note = synthetic
DIQMNQSPSSLSASLGDTITITCRASQNINIWLSWYQQKPGNIPKLFIYKASNLHTGVPSRFSGSGSGTDFTLTISSLQPEDIATYYCQQSQSYPLTFGAGTKLELK

SEQ ID NO: 3 - protein, 5 aa, synthetic construct
  REGION 1..5: note = synthetic
DYYMN

SEQ ID NO: 4 - protein, 19 aa, synthetic construct
  REGION 1..19: note = synthetic
LIRNKAAGYTIYYSASVKG

SEQ ID NO: 5 - protein, 16 aa, synthetic construct
  REGION 1..16: note = synthetic
DMTIYDGYYVDYALDY

SEQ ID NO: 6 - protein, 11 aa, synthetic construct
  REGION 1..11: note = synthetic
RASQNINIWLS

SEQ ID NO: 7 - protein, 7 aa, synthetic construct
  REGION 1..7: note = synthetic
KASNLHT

SEQ ID NO: 8 - protein, 9 aa, synthetic construct
  REGION 1..9: note = synthetic
QQSQSYPLT

SEQ ID NO: 9-16 - skipped (8 entries)

SEQ ID NO: 17 - protein, 119 aa, synthetic construct
  REGION 1..119: note = synthetic
QVQLQQSGPGLMAPSQSLSITCTVSGFSLTSYDVSWIRQSPGKGLEWLGVIWTGGGTDYNSAFMSRLSITKDNSKSQVFLKMSSLQTDDTAIYYCVRVYYDPYYAMDYWGQGTSVTVSS

SEQ ID NO: 18 - protein, 108 aa, synthetic construct
  REGION 1..108: note = synthetic
DIVLTQSPAIMSASPGEKVTMTCSASSSVSSRYLHWYQQKSGASPKLWIYGTSNLASGVPARFSGSGSGTSYSLTISSVEAEDAATYYCQQYHSDPLTFGAGTKLELK

SEQ ID NO: 19 - protein, 5 aa, synthetic construct
  REGION 1..5: note = synthetic
SYDVS

SEQ ID NO: 20 - protein, 16 aa, synthetic construct
  REGION 1..16: note = synthetic
VIWTGGGTDYNSAFMS

SEQ ID NO: 21 - protein, 11 aa, synthetic construct
  REGION 1..11: note = synthetic
VYYDPYYAMDY

SEQ ID NO: 22 - protein, 12 aa, synthetic construct
  REGION 1..12: note = synthetic
SASSSVSSRYLH

SEQ ID NO: 23 - protein, 7 aa, synthetic construct
  REGION 1..7: note = synthetic
GTSNLAS

SEQ ID NO: 24 - protein, 9 aa, synthetic construct
  REGION 1..9: note = synthetic
QQYHSDPLT

SEQ ID NO: 25-30 - skipped (6 entries)

SEQ ID NO: 31 - protein, 117 aa, synthetic construct
  REGION 1..117: note = synthetic
EVQLQQSGPELVKPGASVKMSCKASGYTFTDYHMNWVKRSPGKSLEWIGRINPDYGDARYDQKFKGKATLTVDKSLSTAYMQLNRLTYEDSAVYYCARDGYYVFDYWGQGSTLTVSS

SEQ ID NO: 32 - protein, 112 aa, synthetic construct
  REGION 1..112: note = synthetic
DIVMTQAAFSNPVSLGTSASISCRSSKSLLHSDGITYLSWYLQRPGQSPQLLIYRMSNLVSGVPDRFSGSGSGTDFTLRISRVEAEDVGVYYCAQTLEFPLTFGAGTKLELK

SEQ ID NO: 33 - protein, 5 aa, synthetic construct
  REGION 1..5: note = synthetic
DYHMN

SEQ ID NO: 34 - protein, 17 aa, synthetic construct
  REGION 1..17: note = synthetic
RINPDYGDARYDQKFKG

SEQ ID NO: 35 - protein, 8 aa, synthetic construct
  REGION 1..8: note = synthetic
DGYYVFDY

SEQ ID NO: 36 - protein, 16 aa, synthetic construct
  REGION 1..16: note = synthetic
RSSKSLLHSDGITYLS

SEQ ID NO: 37 - protein, 7 aa, synthetic construct
  REGION 1..7: note = synthetic
RMSNLVS

SEQ ID NO: 38 - protein, 9 aa, synthetic construct
  REGION 1..9: note = synthetic
AQTLEFPLT

SEQ ID NO: 39-45 - skipped (7 entries)

SEQ ID NO: 46 - protein, 116 aa, synthetic construct
  REGION 1..116: note = synthetic
QVQLQQPGTELVKPGASVKLSCKASGYTFTRYWMHWVRQRPGQGLEWIGNINPNIGRANYNEKFKNKATLTVDKSSSTAYMQLSSLTSEDSAVYYCARPGETTDYWGQGTTLTVSS

SEQ ID NO: 47 - protein, 112 aa, synthetic construct
  REGION 1..112: note = synthetic
DVVMTQTPLILSVTIGQPASISCKSSQSLLYTDGKTYLNWLLQRPGQSPKRLIYLVSKLDSGVPDRFSGSGSGTDFTLKISRVEAVDLGVYYCLQSTHYPFTFGTGTKLEIK

SEQ ID NO: 48 - protein, 5 aa, synthetic construct
  REGION 1..5: note = synthetic
RYWMH

SEQ ID NO: 49 - protein, 17 aa, synthetic construct
  REGION 1..17: note = synthetic
NINPNIGRANYNEKFKN

SEQ ID NO: 50 - protein, 7 aa, synthetic construct
  REGION 1..7: note = synthetic
PGETTDY

SEQ ID NO: 51 - protein, 16 aa, synthetic construct
  REGION 1..16: note = synthetic
KSSQSLLYTDGKTYLN

SEQ ID NO: 52 - protein, 7 aa, synthetic construct
  REGION 1..7: note = synthetic
LVSKLDS

SEQ ID NO: 53 - protein, 9 aa, synthetic construct
  REGION 1..9: note = synthetic
LQSTHYPFT

SEQ ID NO: 54-61 - skipped (8 entries)

SEQ ID NO: 62 - protein, 125 aa, synthetic construct
  REGION 1..125: note = synthetic
QVQLQQSGAELVKPGASVRLSCKASGYTFTEFTIHWVKQRSGQGLEWIGWFYPGSDFIKYNEKFKDKATLTADKSSNTVYMELLRLTSEDSAVYFCARHEDRLSDYSYEWYFDVWGAGTAVTVSS

SEQ ID NO: 63 - protein, 107 aa, synthetic construct
  REGION 1..107: note = synthetic
DIQMTQSPASLSASVGETVTITCRASENIDSYLAWYQQKQGKSPQLLVYGATLLADGVPSRFSGSGSGTQYSLKINSLQSEDVAEYFCQHYYSIPLTFGAGTKLELK

SEQ ID NO: 64 - protein, 5 aa, synthetic construct
  REGION 1..5: note = synthetic
EFTIH

SEQ ID NO: 65 - protein, 17 aa, synthetic construct
  REGION 1..17: note = synthetic
WFYPGSDFIKYNEKFKD

SEQ ID NO: 66 - protein, 16 aa, synthetic construct
  REGION 1..16: note = synthetic
HEDRLSDYSYEWYFDV

SEQ ID NO: 67 - protein, 11 aa, synthetic construct
  REGION 1..11: note = synthetic
RASENIDSYLA

SEQ ID NO: 68 - protein, 7 aa, synthetic construct
  REGION 1..7: note = synthetic
GATLLAD

SEQ ID NO: 69 - protein, 9 aa, synthetic construct
  REGION 1..9: note = synthetic
QHYYSIPLT

SEQ ID NO: 70-76 - skipped (7 entries)

SEQ ID NO: 77 - protein, 121 aa, synthetic construct
  REGION 1..121: note = synthetic
QVQLQQSGPELVKPGASVKISCKASGYAFSHSWMNWVKQRPGKGLEWIGWIHLGDGDTYYNGKFKGKATLTADKSSSTAYMQLSSLTSEDSAVYFCARSDGNYVPYAMDYWGQGTSVTVSS

SEQ ID NO: 78 - protein, 107 aa, synthetic construct
  REGION 1..107: note = synthetic
DIVITQTTSSLSASLGDRVTISCRASQDISNYLSWYQQKPDGTVKLLIYYTSRLHSGVPSRFSGSGSGTDYSLTISSLEQEDIATYFCQQDSEHPWTFGGGTELDVR

SEQ ID NO: 79 - protein, 5 aa, synthetic construct
  REGION 1..5: note = synthetic
HSWMN

SEQ ID NO: 80 - protein, 17 aa, synthetic construct
  REGION 1..17: note = synthetic
WIHLGDGDTYYNGKFKG

SEQ ID NO: 81 - protein, 12 aa, synthetic construct
  REGION 1..12: note = synthetic
SDGNYVPYAMDY

SEQ ID NO: 82 - protein, 11 aa, synthetic construct
  REGION 1..11: note = synthetic
RASQDISNYLS

SEQ ID NO: 83 - protein, 7 aa, synthetic construct
  REGION 1..7: note = synthetic
YTSRLHS

SEQ ID NO: 84 - protein, 9 aa, synthetic construct
  REGION 1..9: note = synthetic
QQDSEHPWT

SEQ ID NO: 85-91 - skipped (7 entries)

SEQ ID NO: 92 - protein, 116 aa, synthetic construct
  REGION 1..116: note = synthetic
EVQVQQSGTELVKPGASVKLSCKASGYTFTNYWLHWVKQRPGQGLEWIGNINPSNGGTNYNEKFKTKATLTVDKSSSTAYMQLSSLTSEDSAVYYCARYSTGFAYWGQGTLVTVSA

SEQ ID NO: 93 - protein, 111 aa, synthetic construct
  REGION 1..111: note = synthetic
DIVLTQTTASLAVSLGQRATISCRASESVDTYGASFMHWYQQKPGQPPKLLIYRASNLESGIPARFSGSGSRTDFTLTVNPVEADDVATYYCQQSNEDPRTFGGGTKLEIK

SEQ ID NO: 94 - protein, 5 aa, synthetic construct
  REGION 1..5: note = synthetic
NYWLH

SEQ ID NO: 95 - protein, 17 aa, synthetic construct
  REGION 1..17: note = synthetic
NINPSNGGTNYNEKFKT

SEQ ID NO: 96 - protein, 7 aa, synthetic construct
  REGION 1..7: note = synthetic
YSTGFAY

SEQ ID NO: 97 - protein, 15 aa, synthetic construct
  REGION 1..15: note = synthetic
RASESVDTYGASFMH

SEQ ID NO: 98 - protein, 7 aa, synthetic construct
  REGION 1..7: note = synthetic
RASNLES

SEQ ID NO: 99 - protein, 9 aa, synthetic construct
  REGION 1..9: note = synthetic
QQSNEDPRT

SEQ ID NO: 100-106 - skipped (7 entries)

SEQ ID NO: 107 - protein, 117 aa, synthetic construct
  REGION 1..117: note = synthetic
EVQLQQSGPELVKPGDSVKISCKASGYSFTGYFMNWVMQSHGKSLEWIGRINPYNGDTFYNQKFKGKATLTVDKSSSTAHMELRSLTSEDSAVYYCAGLGHWYFDVWGTGTTVTVSS

SEQ ID NO: 108 - protein, 112 aa, synthetic construct
  REGION 1..112: note = synthetic
DIVMTQSTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 109 - protein, 5 aa, synthetic construct
  REGION 1..5: note = synthetic
GYFMN

SEQ ID NO: 110 - protein, 17 aa, synthetic construct
  REGION 1..17: note = synthetic
RINPYNGDTFYNQKFKG

SEQ ID NO: 111 - protein, 8 aa, synthetic construct
  REGION 1..8: note = synthetic
LGHWYFDV

SEQ ID NO: 112 - protein, 16 aa, synthetic construct
  REGION 1..16: note = synthetic
RSSQSIVHSNGNTYLE

SEQ ID NO: 113 - protein, 7 aa, synthetic construct
  REGION 1..7: note = synthetic
KVSNRFS

SEQ ID NO: 114 - protein, 9 aa, synthetic construct
  REGION 1..9: note = synthetic
FQGSHVPWT

SEQ ID NO: 115-121 - skipped (7 entries)

SEQ ID NO: 122 - protein, 120 aa, synthetic construct
  REGION 1..120: note = synthetic
QVQLEQSGGDLVKPGGSLKLSCAASGFTFSSYGMSWVRQTPDKRLEWVATISSYGSYTSYPDTVKGRFTISRDNAKNTLYLQMSSLKSEDTAMYYCARPDYYVSTGFDYWGQGTTLTVSS

SEQ ID NO: 123 - protein, 107 aa, synthetic construct
  REGION 1..107: note = synthetic
DIVITQSTATLSVTPGDRVSLSCRASQSISDYLHWYQQKSHESPRLLIKYASQSISGIPSRFSGSGSGSDFTLSINSVEPEDVGVYYCQNGHSFPFTFGSGTKLEIK

SEQ ID NO: 124 - protein, 5 aa, synthetic construct
  REGION 1..5: note = synthetic
SYGMS

SEQ ID NO: 125 - protein, 17 aa, synthetic construct
  REGION 1..17: note = synthetic
TISSYGSYTSYPDTVKG

SEQ ID NO: 126 - protein, 11 aa, synthetic construct
  REGION 1..11: note = synthetic
PDYYVSTGFDY

SEQ ID NO: 127 - protein, 11 aa, synthetic construct
  REGION 1..11: note = synthetic
RASQSISDYLH

SEQ ID NO: 128 - protein, 7 aa, synthetic construct
  REGION 1..7: note = synthetic
YASQSIS

SEQ ID NO: 129 - protein, 9 aa, synthetic construct
  REGION 1..9: note = synthetic
QNGHSFPFT

SEQ ID NO: 130-136 - skipped (7 entries)

SEQ ID NO: 137 - protein, 118 aa, synthetic construct
  REGION 1..118: note = synthetic
QVQAQQSGAVLVQPGASVKLSCKASGYTFTDYTIHWIKQRSGQGLEWIGWFYPGSGGLKYHEKFKDKATLTADKSSSTVYMELSRLTSEDSAVYFCTRHGNYDGFAYWGQGTLVTVSA

SEQ ID NO: 138 - protein, 112 aa, synthetic construct
  REGION 1..112: note = synthetic
DIVLTQTPLSLPVSLGDQASISYRSSQSLVHSNGNTYLHWYLQKPGQSPKLLIYKVSNRFSGVPDRFSGSGSGTDFTLKISRVEAEDLGVYFCSQSTHVPFTFGSGTKLEIK

SEQ ID NO: 139 - protein, 5 aa, synthetic construct
  REGION 1..5: note = synthetic
DYTIH

SEQ ID NO: 140 - protein, 17 aa, synthetic construct
  REGION 1..17: note = synthetic
WFYPGSGGLKYHEKFKD

SEQ ID NO: 141 - protein, 9 aa, synthetic construct
  REGION 1..9: note = synthetic
HGNYDGFAY

SEQ ID NO: 142 - protein, 16 aa, synthetic construct
  REGION 1..16: note = synthetic
RSSQSLVHSNGNTYLH

SEQ ID NO: 143 - protein, 7 aa, synthetic construct
  REGION 1..7: note = synthetic
KVSNRFS

SEQ ID NO: 144 - protein, 9 aa, synthetic construct
  REGION 1..9: note = synthetic
SQSTHVPFT

SEQ ID NO: 145-148 - skipped (4 entries)

SEQ ID NO: 149 - protein, 127 aa, synthetic construct
  REGION 1..127: note = synthetic
EVQLVESGGGLVKPGGSLRLSCAASGFAFGDYYMNWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 150 - protein, 107 aa, synthetic construct
  REGION 1..107: note = synthetic
DIVMTQSPSSVSASVGDRVTITCRASRNINIWLSWYQQKPGKAPKLFIYKASNLHTGVPSRFSGSGSGTDFTLTISSLQPEDFATYYCLQSQSYPLTFGQGTKLEIK

SEQ ID NO: 151 - protein, 19 aa, synthetic construct
  REGION 1..19: note = synthetic
LIRNKAAGYTIYYSAPVKG

SEQ ID NO: 152 - protein, 11 aa, synthetic construct
  REGION 1..11: note = synthetic
RASRNINIWLS

SEQ ID NO: 153 - protein, 9 aa, synthetic construct
  REGION 1..9: note = synthetic
LQSQSYPLT

SEQ ID NO: 154 - protein, 30 aa, synthetic construct
  REGION 1..30: note = synthetic
EVQLVESGGGLVKPGGSLRLSCAASGFAFG

SEQ ID NO: 155 - protein, 14 aa, synthetic construct
  REGION 1..14: note = synthetic
WVRQAPGKGLEWVA

SEQ ID NO: 156 - protein, 32 aa, synthetic construct
  REGION 1..32: note = synthetic
RFTISRDDSKSTLYLQMNSLKTEDTAVYYCVR

SEQ ID NO: 157 - protein, 11 aa, synthetic construct
  REGION 1..11: note = synthetic
WGQGTLVTVSS

SEQ ID NO: 158 - protein, 23 aa, synthetic construct
  REGION 1..23: note = synthetic
DIVMTQSPSSVSASVGDRVTITC

SEQ ID NO: 159 - protein, 15 aa, synthetic construct
  REGION 1..15: note = synthetic
WYQQKPGKAPKLFIY

SEQ ID NO: 160 - protein, 32 aa, synthetic construct
  REGION 1..32: note = synthetic
GVPSRFSGSGSGTDFTLTISSLQPEDFATYYC

SEQ ID NO: 161 - protein, 10 aa, synthetic construct
  REGION 1..10: note = synthetic
FGQGTKLEIK

SEQ ID NO: 162 - protein, 127 aa, synthetic construct
  REGION 1..127: note = synthetic
QVQLQESGGGLVKPGGSLRLSCAASGFAFTDYYMNWIRQAPGKGLEWVSLIRNKAAGYTIYYADSVKGRFTISRDNAKSSLYLQMNSLRAEDTAVYYCVRDMTIYDGYYVDYALDYWGQGTLVTVSS

SEQ ID NO: 163 - protein, 107 aa, synthetic construct
  REGION 1..107: note = synthetic
EIVMTQSPATLSVSPGERATLSCRASQNINIWLSWYQQKPGQAPRLFIYKASNLHTGVPARFSGSGSGTEFTLTISSLQSEDFAVYYCLQSQSYPLTFGQGTKLEIK

SEQ ID NO: 164 - protein, 19 aa, synthetic construct
  REGION 1..19: note = synthetic
LIRNKAAGYTIYYADSVKG

SEQ ID NO: 165 - protein, 30 aa, synthetic construct
  REGION 1..30: note = synthetic
QVQLQESGGGLVKPGGSLRLSCAASGFAFT

SEQ ID NO: 166 - protein, 14 aa, synthetic construct
  REGION 1..14: note = synthetic
WIRQAPGKGLEWVS

SEQ ID NO: 167 - protein, 32 aa, synthetic construct
  REGION 1..32: note = synthetic
RFTISRDNAKSSLYLQMNSLRAEDTAVYYCVR

SEQ ID NO: 168 - protein, 23 aa, synthetic construct
  REGION 1..23: note = synthetic
EIVMTQSPATLSVSPGERATLSC

SEQ ID NO: 169 - protein, 15 aa, synthetic construct
  REGION 1..15: note = synthetic
WYQQKPGQAPRLFIY

SEQ ID NO: 170 - protein, 32 aa, synthetic construct
  REGION 1..32: note = synthetic
GVPARFSGSGSGTEFTLTISSLQSEDFAVYYC

SEQ ID NO: 171 - protein, 127 aa, synthetic construct
  REGION 1..127: note = synthetic
QVQLVESGGGLVKPGGSLRLSCAASGFAFTDYYMNWIRQAPGKGLEWVALIRNKAAGYTIYYSDSVKGRFTISRDNAKSSLYLQMNSLRAEDTAVYYCVRDMTIYDGYYVDYALDYWGQGTLVTVSS

SEQ ID NO: 172 - protein, 107 aa, synthetic construct
  REGION 1..107: note = synthetic
DIVMTQSPSSVSASVGDRVTITCRASQNINIWLSWYQQKPGKAPKLFIYKASNLHTGVPSRFSGSGSGTDFTLTISSLQPEDFATYYCLQSQSYPLTFGQGTKLEIK

SEQ ID NO: 173 - protein, 19 aa, synthetic construct
  REGION 1..19: note = synthetic
LIRNKAAGYTIYYSDSVKG

SEQ ID NO: 174 - protein, 30 aa, synthetic construct
  REGION 1..30: note = synthetic
QVQLVESGGGLVKPGGSLRLSCAASGFAFT

SEQ ID NO: 175 - protein, 14 aa, synthetic construct
  REGION 1..14: note = synthetic
WIRQAPGKGLEWVA

SEQ ID NO: 176 - protein, 127 aa, synthetic construct
  REGION 1..127: note = synthetic
EVQLVESGGGLVKPGGSLRLSCAASGFAFTDYYMNWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 177 - protein, 107 aa, synthetic construct; identical to SEQ ID NO: 172
  REGION 1..107: note = synthetic

SEQ ID NO: 178 - protein, 30 aa, synthetic construct
  REGION 1..30: note = synthetic
EVQLVESGGGLVKPGGSLRLSCAASGFAFT

SEQ ID NO: 179 - protein, 127 aa, synthetic construct
  REGION 1..127: note = synthetic
EVQLVESGGGLVKPGGSLRLSCAASGFDFTDYYMHWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 180 - protein, 5 aa, synthetic construct
  REGION 1..5: note = synthetic
DYYMH

SEQ ID NO: 181 - protein, 30 aa, synthetic construct
  REGION 1..30: note = synthetic
EVQLVESGGGLVKPGGSLRLSCAASGFDFT

SEQ ID NO: 182 - protein, 127 aa, synthetic construct
  REGION 1..127: note = synthetic
EVQLVESGGGLVKPGGSLRLSCAASGFDFTDYYMNWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 183 - protein, 127 aa, synthetic construct
  REGION 1..127: note = synthetic
EVQLVESGGGLVKPGGSLRLSCAASGFAFTDYYLRWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 184 - protein, 5 aa, synthetic construct
  REGION 1..5: note = synthetic
DYYLR

SEQ ID NO: 185 - protein, 127 aa, synthetic construct
  REGION 1..127: note = synthetic
EVQLVESGGGLVKPGGSLRLSCAASGFNFTDYYMNWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 186 - protein, 30 aa, synthetic construct
  REGION 1..30: note = synthetic
EVQLVESGGGLVKPGGSLRLSCAASGFNFT

SEQ ID NO: 187 - protein, 127 aa, synthetic construct
  REGION 1..127: note = synthetic
EVQLVESGGGLVKPGGSLRLSCAASGFAFTDYYLSWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 188 - protein, 5 aa, synthetic construct
  REGION 1..5: note = synthetic
DYYLS

SEQ ID NO: 189 - protein, 127 aa, synthetic construct
  REGION 1..127: note = synthetic
EVQLVESGGGLVKPGGSLRLSCAASGYAFTDYYMNWVRQAPGKGLEWVALIRNKAAGYTIYYSAPVKGRFTISRDDSKSTLYLQMNSLKTEDTAVYYCVRDMTIYDGYYVDYALDYWGQGTLVTVSS

SEQ ID NO: 190 - protein, 30 aa, synthetic construct
  REGION 1..30: note = synthetic
EVQLVESGGGLVKPGGSLRLSCAASGYAFT

SEQ ID NO: 191 - skipped

SEQ ID NO: 192 - protein, 19 aa, synthetic construct
  REGION 1..19: note = synthetic
  SITE 14: note = misc_feature - Xaa can be S or A
  SITE 15: note = misc_feature - Xaa can be A or D
  SITE 16: note = misc_feature - Xaa can be S or P
LIRNKAAGYTIYYXXXVKG

SEQ ID NO: 193 - protein, 11 aa, synthetic construct
  REGION 1..11: note = synthetic
  SITE 4: note = misc_feature - Xaa can be Q or R
RASXNINIWLS

SEQ ID NO: 194 - protein, 9 aa, synthetic construct
  REGION 1..9: note = synthetic
  SITE 1: note = misc_feature - Xaa can be Q or L
XQSQSYPLT

SEQ ID NO: 195 - protein, 30 aa, synthetic construct
  REGION 1..30: note = synthetic
  SITE 1: note = misc_feature - Xaa can be Q or E
  SITE 5: note = misc_feature - Xaa can be Q or V
  SITE 27: note = misc_feature - Xaa can be F or Y
  SITE 28: note = misc_feature - Xaa can be A, D or N
  SITE 30: note = misc_feature - Xaa can be T or G
XVQLXESGGGLVKPGGSLRLSCAASGXXFX

SEQ ID NO: 196 - protein, 14 aa, synthetic construct
  REGION 1..14: note = synthetic
  SITE 2: note = misc_feature - Xaa can be I or V
  SITE 14: note = misc_feature - Xaa can be S or A
WXRQAPGKGLEWVX

SEQ ID NO: 197 - protein, 32 aa, synthetic construct
  REGION 1..32: note = synthetic
  SITE 8: note = misc_feature - Xaa can be N or D
  SITE 9: note = misc_feature - Xaa can be A or S
  SITE 12: note = misc_feature - Xaa can be S or T
  SITE 21: note = misc_feature - Xaa can be R or K
  SITE 22: note = misc_feature - Xaa can be A or T
RFTISRDXXKSXLYLQMNSLXXEDTAVYYCVR

SEQ ID NO: 198 - protein, 23 aa, synthetic construct
  REGION 1..23: note = synthetic
  SITE 1: note = misc_feature - Xaa can be D or E
  SITE 9: note = misc_feature - Xaa can be S or A
  SITE 10: note = misc_feature - Xaa can be S or T
  SITE 11: note = misc_feature - Xaa can be L or V
  SITE 13: note = misc_feature - Xaa can be A or V
  SITE 15: note = misc_feature - Xaa can be V or P
  SITE 17: note = misc_feature - Xaa can be D or E
  SITE 19: note = misc_feature - Xaa can be V or A
  SITE 21: note = misc_feature - Xaa can be I or L
  SITE 22: note = misc_feature - Xaa can be T or S
XIVMTQSPXXXSXSXGXRXTXXC

SEQ ID NO: 199 - protein, 15 aa, synthetic construct
  REGION 1..15: note = synthetic
  SITE 8: note = misc_feature - Xaa can be Q or K
  SITE 11: note = misc_feature - Xaa can be K or R
WYQQKPGXAPXLFIY

SEQ ID NO: 200 - protein, 32 aa, synthetic construct
  REGION 1..32: note = synthetic
  SITE 4: note = misc_feature - Xaa can be S or A
  SITE 14: note = misc_feature - Xaa can be D or E
  SITE 24: note = misc_feature - Xaa can be S or P
  SITE 29: note = misc_feature - Xaa can be T or V
GVPXRFSGSGSGTXFTLTISSLQXEDFAXYYC

SEQ ID NO: 201 - protein, 327 aa, synthetic construct
  REGION 1..327: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - protein, 107 aa, synthetic construct [sequence omitted]

SEQ ID NO: 203 - protein, 327 aa, synthetic construct
  REGION 1..327: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - protein, 327 aa, synthetic construct
  REGION 1..327: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 205 - protein, 119 aa, synthetic construct [sequence omitted]

SEQ ID NO: 206 - protein, 108 aa, synthetic construct [sequence omitted]

SEQ ID NO: 207 - protein, 120 aa, synthetic construct
  REGION 1..120: note = synthetic
QVQLVQSGAEVKKPGASVKVSCKASGYTFTNYWMNWVRQAPRQGLEWMGMFHPTGDVTRLNQKFKDRVTMTRDTSTSTVYMELSSLRSEDTAVYYCARTTSMIIGGFAYWGQGTLVTVSS

SEQ ID NO: 208 - protein, 112 aa, synthetic construct
  REGION 1..112: note = synthetic
DIVMTQTPLSLSVTPGQPASISCRSSKSLLHRNAITYFYWYLHKPGQPPQLLIYQMSNLASGVPDRFSGSGSGTDFTLKISRVEAEDVGVYYCAQNLELPLTFGGGTKVEIK

SEQ ID NO: 209 - protein, 118 aa, synthetic construct
  REGION 1..118: note = synthetic
EVQLVESGGDLVQPGRSLRLSCAASGFTFDDYAMHWVRQAPGKGLEWVSVISWNSDVIAYSDSVKGRFTISRDNAKNSLYLQMNSLRTEDTALYYCTKGHKWSFFDYWGQGTLVTVSS

SEQ ID NO: 210 - protein, 107 aa, synthetic construct
  REGION 1..107: note = synthetic
EIVLTQSPATLSLSPGERATLSCRASQSISSYLAWYQQKPGQAPRLLIFNVANRATDIPARFSGSGSGTDFTLTISSLEPEDFAVYYCQQRSNWPLTFGGGTKVEIK

SEQ ID NO: 211 - protein, 575 aa, synthetic construct
  REGION 1..575: note = synthetic
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 212 - protein, 570 aa, synthetic construct
  REGION 1..570: note = synthetic
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

(19) United States
(12) Patent Application Publication
(10) Pub. No.: US 2025/0002593 A1
(43) Pub. Date: Jan. 2, 2025

(54) NOVEL ANTI-IL-36R ANTIBODIES

(71) Applicant: INMAGENE PTE. LTD., Singapore (SG)

(72) Inventors: Pengcheng FAN, Zhejiang, Hangzhou (CN); Run LEI, Shanghai (CN); Chongtian GUO, Shanghai (CN); Lihua FAN, Zhejiang, Hangzhou (CN); Qiang SUN, Zhejiang, Hangzhou (CN); Zhihao XU, Zhejiang, Hangzhou (CN)

(21) Appl. No.: 18/705,557

(22) PCT Filed: Oct. 27, 2022
(86) PCT No.: PCT/CN2022/127898

§ 371 Date: Apr. 28, 2024

(30) Foreign Application Priority Data
Oct. 29, 2021 (WO) PCT/CN2021/127544
Sep. 13, 2022 (WO) PCT/CN2022/118446

(51) Int. Cl. C07K 16/28; A61K 39/00; C07K 14/725; G01N 33/68

(52) U.S. Cl. CPC C07K 16/2866; A61K 39/4633; A61K 39/46433; C07K 14/7051; G01N 33/6869; A61K 2039/54; A61K 2039/542; A61K 2039/543; C07K 2317/24; C07K 2317/31; C07K 2317/35; C07K 2317/569; C07K 2317/622; C07K 2317/92; G01N 2333/7155

(57) ABSTRACT

Provided are anti-IL-36R antibodies or antigen-binding fragments thereof, isolated polynucleotides encoding the same, pharmaceutical compositions comprising the same and the uses thereof.

### FIELD OF THE INVENTION

The present disclosure generally relates to novel anti-IL-36R antibodies.

### BACKGROUND

Interleukin 36 receptor (IL-36R) is expressed on cell surface and belongs to the IL1R family. There are three agonists of IL-36R (namely, IL-36α, IL-36β, and IL-36γ), and two antagonists (namely, IL-36Ra (IL36RN) and IL-38). Ligands of IL-36R are mainly from keratinocytes, epithelial cells, T/B lymphocytes, monocytes, dendritic cells, macrophages etc. IL-36R is usually expressed on keratinocytes, fibroblasts, dendritic cells, endothelial cells, monocytes, macrophages, Langerhans cells, CD4+ T cells etc., and functions by signaling through NFκB or MARK pathway. Key functions of IL-36R in a cell is to induce proinflammatory cytokines and chemokines, and to promote cell activation and proliferation.

However, IL-36R-related dermatologic diseases have high unmet medical needs. Therefore, needs remain for novel anti-IL-36R antibodies.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides an antibody or an antigen-binding fragment thereof capable of specifically binding to human IL-36R, comprising heavy chain complementary determining region 1 (HCDR1), HCDR2 and HCDR3, wherein the HCDR1 comprises an amino acid sequence of DYYX₁X₂ (SEQ ID NO: 191), SEQ ID NO: 19, 33, 48, 64, 79, 94, 109, 124 or 139; the HCDR2 comprises an amino acid sequence of LIRNKAAGYTIYYX₃X₄X₅VKG (SEQ ID NO: 192), SEQ ID NO: 20, 34, 49, 65, 80, 95, 110, 125 or 140; and the HCDR3 comprises an amino acid sequence of SEQ ID NO: 5, 21, 35, 50, 66, 81, 96, 111, 126 or 141; wherein, X₁ is M or L; X₂ is N, H, S or R; X₃ is S or A; X₄ is A or D; X₅ is S or P.

In some embodiments, the antibody or an antigen-binding fragment thereof comprises light chain complementary determining region 1 (LCDR1), LCDR2 and LCDR3, wherein the LCDR1 comprises an amino acid sequence of RASX₁₈NINIWLS (SEQ ID NO: 193), SEQ ID NO: 22, 36, 51, 67, 82, 97, 112, 127 or 142; the LCDR2 comprises an amino acid sequence of SEQ ID NO: 7, 23, 37, 52, 68, 83, 98, 113, 128 or 113; and the LCDR3 comprises an amino acid sequence of X₁₉QSQSYPLT (SEQ ID NO: 194), SEQ ID NO: 24, 38, 53, 69, 84, 99, 114, 129 or 143; wherein, X₁₈ is Q or R; X₁₉ is Q or L.

In some embodiments, the antibody or an antigen-binding fragment thereof comprises a heavy chain variable region comprising HCDR1, HCDR2 and HCDR3, and/or a light chain variable region comprising LCDR1, LCDR2 and LCDR3, wherein (a) the HCDR1 comprises an amino acid sequence of DYYX₁X₂ (SEQ ID NO: 191), the HCDR2 comprises an amino acid sequence of LIRNKAAGYTIYYX₃X₄X₅VKG (SEQ ID NO: 192), the HCDR3 comprises an amino acid sequence of SEQ ID NO: 5, the LCDR1 comprises an amino acid sequence of RASX₁₈NINIWLS (SEQ ID NO: 193), the LCDR2 comprises an amino acid sequence of SEQ ID NO: 7, and the LCDR3 comprises an amino acid sequence of X₁₉QSQSYPLT (SEQ ID NO: 194), wherein, X₁ is M or L; X₂ is N, H, S or R; X₃ is S or A; X₄ is A or D; X₅ is S or P; X₁₈ is Q or R; X₁₉ is Q or L; (b) the HCDR1 comprises an amino acid sequence of SEQ ID NO: 19, the HCDR2 comprises an amino acid sequence of SEQ ID NO: 20, the HCDR3 comprises an amino acid sequence of SEQ ID NO: 21, the LCDR1 comprises an amino acid sequence of SEQ ID NO: 22, the LCDR2 comprises an amino acid sequence of SEQ ID NO: 23, and the LCDR3 comprises an amino acid sequence of SEQ ID NO: 24; (c) the HCDR1 comprises an amino acid sequence of SEQ ID NO: 33, the HCDR2 comprises an amino acid sequence of SEQ ID NO: 34, the HCDR3 comprises an amino acid sequence of SEQ ID NO: 35, the LCDR1 comprises an amino acid sequence of SEQ ID NO: 36, the LCDR2 comprises an amino acid sequence of SEQ ID NO: 37, and the LCDR3 comprises an amino acid sequence of SEQ ID NO: 38; (d) the HCDR1 comprises an amino acid sequence of SEQ ID NO: 48, the HCDR2 comprises an amino acid sequence of SEQ ID NO: 49, the HCDR3 comprises an amino acid sequence of SEQ ID NO: 50, the LCDR1 comprises an amino acid sequence of SEQ ID NO: 51, the LCDR2 comprises an amino acid sequence of SEQ ID NO: 52, and the LCDR3 comprises an amino acid sequence of SEQ ID NO: 53, (e) the HCDR1 comprises an amino acid sequence of SEQ ID NO: 64, the HCDR2 comprises an amino acid sequence of SEQ ID NO: 65, the HCDR3 comprises an amino acid sequence of SEQ ID NO: 66, the LCDR1 comprises an amino acid sequence of SEQ ID NO: 67, the LCDR2 comprises an amino acid sequence of SEQ ID NO: 68, and the LCDR3 comprises an amino acid sequence of SEQ ID NO: 69; (f) the HCDR1 comprises an amino acid sequence of SEQ ID NO: 79, the HCDR2 comprises an amino acid sequence of SEQ ID NO: 80, the HCDR3 comprises an amino acid sequence of SEQ ID NO: 81, the LCDR1 comprises an amino acid sequence of SEQ ID NO: 82, the LCDR2 comprises an amino acid sequence of SEQ ID NO: 83, and the LCDR3 comprises an amino acid sequence of SEQ ID NO: 84; (g) the HCDR1 comprises an amino acid sequence of SEQ ID NO: 94, the HCDR2 comprises an amino acid sequence of SEQ ID NO: 95, the HCDR3 comprises an amino acid sequence of SEQ ID NO: 96, the LCDR1 comprises an amino acid sequence of SEQ ID NO: 97, the LCDR2 comprises an amino acid sequence of SEQ ID NO: 98, and the LCDR3 comprises an amino acid sequence of SEQ ID NO: 99; (h) the HCDR1 comprises an amino acid sequence of SEQ ID NO: 109, the HCDR2 comprises an amino acid sequence of SEQ ID NO: 110, the HCDR3 comprises an amino acid sequence of SEQ ID NO: 111, the LCDR1 comprises an amino acid sequence of SEQ ID NO: 112, the LCDR2 comprises an amino acid sequence of SEQ ID NO: 113, and the LCDR3 comprises an amino acid sequence of SEQ ID NO: 114; (i) the HCDR1 comprises an amino acid sequence of SEQ ID NO: 124, the HCDR2 comprises an amino acid sequence of SEQ ID NO: 125, the HCDR3 comprises an amino acid sequence of SEQ ID NO: 126, the LCDR1 comprises an amino acid sequence of SEQ ID NO: 127, the LCDR2 comprises an amino acid sequence of SEQ ID NO: 128, and the LCDR3 comprises an amino acid sequence of SEQ ID NO: 129; or (j) the HCDR1 comprises an amino acid sequence of SEQ ID NO: 139, the HCDR2 comprises an amino acid sequence of SEQ ID NO: 140, the HCDR3 comprises an amino acid sequence of SEQ ID NO: 141, the LCDR1 comprises an amino acid sequence of SEQ ID NO: 142, the LCDR2 comprises an amino acid sequence of SEQ ID NO: 113, and the LCDR3 comprises an amino acid sequence of SEQ ID NO: 143.

In some embodiments, the HCDR1 comprises an amino acid sequence of SEQ ID NO: 3, 180, 184 or 188, the HCDR2 comprises an amino acid sequence of SEQ ID NO: 4, 151, 164 or 173, the HCDR3 comprises an amino acid sequence of SEQ ID NO: 5, the LCDR1 comprises an amino acid sequence of SEQ ID NO: 6 or 152, the LCDR2 comprises an amino acid sequence of SEQ ID NO: 7, and the LCDR3 comprises an amino acid sequence of SEQ ID NO: 8 or 153.

In some embodiments, (a) the HCDR1 comprises an amino acid sequence of SEQ ID NO: 3; the HCDR2 comprises an amino acid sequence of SEQ ID NO: 4; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 8; (b) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153; (c) the HCDR1 comprises an amino acid sequence of SEQ ID NO: 3; the HCDR2 comprises an amino acid sequence of SEQ ID NO: 164; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 153; (d) the HCDR1 comprises an amino acid sequence of SEQ ID NO: 3; the HCDR2 comprises an amino acid sequence of SEQ ID NO: 173; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 153; (e) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 153; (f) the HCDR1 comprises an amino acid sequence of SEQ ID NO: 180; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153; (g) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153; (h) the HCDR1 comprises an amino acid sequence of SEQ ID NO: 184; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153; (i) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153; (j) the HCDR1 comprises an amino acid sequence of SEQ ID NO: 188; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153; or (k) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153.

In some embodiments, the antibody or an antigen-binding fragment thereof provided herein further comprises one or more of heavy chain framework region 1 (HFR1), HFR2, HFR3 and HFR4, and/or one or more of light chain framework region 1 (LFR1), LFR2, LFR3 and LFR4, wherein the HFR1 comprises an amino acid sequence of X₆VQLX₇ESGGGLVKPGGSLRLSCAASGX₈X₉FX₁₀ (SEQ ID NO: 195), or a homologous sequence of at least 85% sequence identity thereof, the HFR2 comprises an amino acid sequence of WX₁₁RQAPGKGLEWVX₁₂ (SEQ ID NO: 196), or a homologous sequence of at least 85% sequence identity thereof, the HFR3 comprises an amino acid sequence of RFTISRDX₁₃X₁₄KSX₁₅LYLQMNSLX₁₆X₁₇EDTAVYYCVR (SEQ ID NO: 197), or a homologous sequence of at least 85% sequence identity thereof, the HFR4 comprises an amino acid sequence of SEQ ID NO: 157, or a homologous sequence of at least 85% sequence identity thereof, the LFR1 comprises an amino acid sequence of X₂₀IVMTQSPX₂₁X₂₂X₂₃SX₂₄SX₂₅GX₂₆RX₂₇TX₂₈X₂₉C (SEQ ID NO: 198), or a homologous sequence of at least 85% sequence identity thereof, the LFR2 comprises an amino acid sequence of WYQQKPGX₃₀APX₃₁LFIY (SEQ ID NO: 199), or a homologous sequence of at least 85% sequence identity thereof, the LFR3 comprises an amino acid sequence of GVPX₃₂RFSGSGSGTX₃₃FTLTISSLQX₃₄EDFAX₃₅YYC (SEQ ID NO: 200), or a homologous sequence of at least 85% sequence identity thereof, and the LFR4 comprises an amino acid sequence of SEQ ID NO: 161, or a homologous sequence of at least 85% sequence identity thereof, wherein, X₆ is Q or E; X₇ is Q or V; X₈ is F or Y; X₉ is A, D or N; X₁₀ is T or G; X₁₁ is I or V; X₁₂ is S or A; X₁₃ is N or D; X₁₄ is A or S; X₁₅ is S or T; X₁₆ is R or K; X₁₇ is A or T; X₂₀ is D or E; X₂₁ is S or A; X₂₂ is S or T; X₂₃ is L or V; X₂₄ is A or V; X₂₅ is V or P; X₂₆ is D or E; X₂₇ is V or A; X₂₈ is I or L; X₂₉ is T or S, X₃₀ is Q or K; X₃₁ is K or R; X₃₂ is S or A; X₃₃ is D or E; X₃₄ is S or P; X₃₅ is T or V.

In some embodiments, the HFR1 comprises a sequence selected from the group consisting of SEQ ID NOs: 9, 25, 39, 54, 70, 85, 100, 115, 130, 144, 154, 165, 174, 178, 181, 186, 190 and 195, the HFR2 comprises a sequence selected from the group consisting of SEQ ID NOs: 10, 26, 40, 55, 71, 86, 101, 116, 131, 145, 155, 166, 175, and 196, the HFR3 comprises the sequence selected from the group consisting of SEQ ID NOs: 11, 27, 41, 56, 72, 87, 56, 117, 132, 146, 156, 167, 167, 156, 156, 156, 156, 156, 156, 156 and 197, the HFR4 comprises the sequence selected from the group consisting of SEQ ID NOs: 12, 42, 57, 73, 102, 118, and 157, the LFR1 comprises the sequence from the group consisting of SEQ ID NOs: 13, 28, 43, 58, 74, 88, 103, 119, 133, 147, 158, 168 and 198, the LFR2 comprises the sequence selected from the group consisting of SEQ ID NOs: 14, 29, 44, 59, 75, 89, 104, 120, 134, 159, 169 and 199, the LFR3 comprises a sequence selected from the group consisting of SEQ ID NOs: 15, 30, 45, 60, 76, 90, 105, 121, 135, 148, 160, 170 and 200, and the LFR4 comprises a sequence selected from the group consisting of SEQ ID NOs: 16, 61, 91, 106, 136, 161.

In some embodiments, the heavy chain variable region of the antibody or an antigen-binding fragment thereof provided herein comprises the sequence selected from the group consisting of SEQ ID NOs: 1, 17, 31, 46, 62, 77, 92, 107, 122, 137, 149, 162, 171, 176, 179, 182, 183, 185, 187 and 189, and a homologous sequence thereof having at least 80% sequence identity yet retaining specific binding affinity to human IL-36R.

In some embodiments, the light chain variable region of the antibody or an antigen-binding fragment thereof provided herein comprises the sequence selected from the group consisting of SEQ ID NOs: 2, 18, 32, 47, 63, 78, 93, 108, 123, 138, 150, 163, 172 and 177, and a homologous sequence thereof having at least 80% sequence identity yet retaining specific binding affinity to human IL-36R.

In some embodiments, in the antibody or an antigen-binding fragment thereof provided herein, the heavy chain variable region comprises the sequence of SEQ ID NO: 1 and the light chain variable region comprises the sequence of SEQ ID NO: 2; or the heavy chain variable region comprises the sequence of SEQ ID NO: 17 and the light chain variable region comprises the sequence of SEQ ID NO: 18; or the heavy chain variable region comprises the sequence of SEQ ID NO: 31 and the light chain variable region comprises the sequence of SEQ ID NO: 32; or the heavy chain variable region comprises the sequence of SEQ ID NO: 46 and the light chain variable region comprises the sequence of SEQ ID NO: 47; or the heavy chain variable region comprises the sequence of SEQ ID NO: 62 and the light chain variable region comprises the sequence of SEQ ID NO: 63; or the heavy chain variable region comprises the sequence of SEQ ID NO: 77 and the light chain variable region comprises the sequence of SEQ ID NO: 78; or the heavy chain variable region comprises the sequence of SEQ ID NO: 92 and the light chain variable region comprises the sequence of SEQ ID NO: 93; or the heavy chain variable region comprises the sequence of SEQ ID NO: 107 and the light chain variable region comprises the sequence of SEQ ID NO: 108; or the heavy chain variable region comprises the sequence of SEQ ID NO: 122 and the light chain variable region comprises the sequence of SEQ ID NO: 123; or the heavy chain variable region comprises the sequence of SEQ ID NO: 137 and the light chain variable region comprises the sequence of SEQ ID NO: 138; or the heavy chain variable region comprises the sequence of SEQ ID NO: 149 and the light chain variable region comprises the sequence of SEQ ID NO: 150; or the heavy chain variable region comprises the sequence of SEQ ID NO: 162 and the light chain variable region comprises the sequence of SEQ ID NO: 163; or the heavy chain variable region comprises the sequence of SEQ ID NO: 171 and the light chain variable region comprises the sequence of SEQ ID NO: 172; or the heavy chain variable region comprises the sequence of SEQ ID NO: 176 and the light chain variable region comprises the sequence of SEQ ID NO: 177; or the heavy chain variable region comprises the sequence of SEQ ID NO: 179 and the light chain variable region comprises the sequence of SEQ ID NO: 150; or the heavy chain variable region comprises the sequence of SEQ ID NO: 182 and the light chain variable region comprises the sequence of SEQ ID NO: 150; or the heavy chain variable region comprises the sequence of SEQ ID NO: 183 and the light chain variable region comprises the sequence of SEQ ID NO: 150; or the heavy chain variable region comprises the sequence of SEQ ID NO: 185 and the light chain variable region comprises the sequence of SEQ ID NO: 150; or the heavy chain variable region comprises the sequence of SEQ ID NO: 187 and the light chain variable region comprises the sequence of SEQ ID NO: 150; or the heavy chain variable region comprises the sequence of SEQ ID NO: 189 and the light chain variable region comprises the sequence of SEQ ID NO: 150.

In some embodiments, the antibody or an antigen-binding fragment thereof provided herein further comprises one or more amino acid residue substitutions or modifications yet retains specific binding affinity to human IL-36R. In some embodiments, at least one of the substitutions or modifications is in one or more of the CDR sequences, and/or in one or more of the non-CDR sequences of the heavy chain variable region or light chain variable region. In some embodiments, at least one of the substitutions is a conservative substitution.

In some embodiments, the antibody or an antigen-binding fragment thereof provided herein further comprises an Fc region, optionally an Fc region of human immunoglobulin (Ig), or optionally an Fc region of human IgG. In some embodiments, the Fc region is derived from human IgG1, IgG2, IgG3, IgG4, IgA1, IgA2 or IgM. In some embodiments, the Fc region derived from human IgG4 comprises mutations of M252Y/S254T/T256E (YTE). In some embodiments, the Fc region derived from human IgG4 comprises mutations of T307Q/N434A (QA).

In some embodiments, the antibody or an antigen-binding fragment thereof provided herein is humanized. In some embodiments, the antibody or an antigen-binding fragment thereof provided herein is a monoclonal antibody, a bispecific antibody, a multi-specific antibody, a recombinant antibody, a chimeric antibody, a labeled antibody, a bivalent antibody, an anti-idiotypic antibody or a fusion protein.

In some embodiments, the antibody or an antigen-binding fragment thereof provided herein is a diabody, an Fab, an Fab′, a F(ab′)₂, an Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In some embodiments, the antibody or an antigen-binding fragment thereof provided herein has one or more binding properties to human IL-36R selected from the group consisting of: a) having a binding affinity to human IL-36R with a K_{d} of no more than 2E-08 M (preferably no more than 1E-08 M, for example, no more than 9E-09M, 8E-09M, 7E-09M, 6E-09M, 5E-09M, 4E-09M, 3E-09M, 2E-09M, or 1E-10 M) as measured by Biolayer interferometry (BLI) assay, b) having a binding affinity to human IL-36R with a K_{d} of no more than 1E-08 M (for example, no more than 9E-09M, 8E-09M, 7E-09M, 6E-09M, 5E-09M, 4E-09M, 3E-09M, 2E-09M, or 1E-10 M) as measured by Surface Plasmon Resonance (SPR) assay and c) specifically binding to human IL-36R with an EC₅₀ of no more than 0.1 μg/mL (for example, 0.09 μg/mL, 0.08 μg/mL, 0.07 μg/mL, 0.06 μg/mL, 0.05 μg/mL, 0.04 μg/mL, 0.03 μg/mL, 0.02 μg/mL, 0.01 μg/mL, or 0.005 μg/mL) as measured by enzyme linked immunosorbent assay (ELISA) assay; and d) having a binding affinity to membrane human IL-36R with an EC₅₀ of no more than 0.5 μg/mL (for example, 0.4 μg/mL, 0.3 μg/mL, 0.2 g/mL, 0.19 μg/mL, 0.18 μg/mL, 0.17 μg/mL, 0.16 μg/mL, 0.15 μg/mL, or 0.1 g/mL) as measured by Fluorescence activated Cell Sorting (FACS) assay.

In some embodiments, the antibody or an antigen-binding fragment thereof provided herein is capable of blocking IL-36R signaling induced by an IL-36R agonist (preferably IL-36α), as measured by IL-36R reporter assay.

In some embodiments, the antibody or an antigen-binding fragment thereof provided herein has one or more properties selected from the group consisting of: a) capable of inhibiting IL-36R agonist-induced IL-8 release in a cell, b) having the ability of inhibiting IL-36R agonist-induced IL-6 release in a cell, and d) having the ability of inhibiting IL-36R agonist-induced TNF-α release in a cell; wherein the IL-36R agonist includes IL-36α, IL-36β, and/or IL-36γ.

In another aspect, the prevent disclosure provides an anti-IL-36R antibody or an antigen-binding fragment thereof that does not compete for binding to human IL-36R with the antibody or an antigen-binding fragment thereof in the art. In some embodiments, the antibody or an antigen-binding fragment thereof does not compete for binding to human IL-36R with an antibody comprising a heavy chain variable region comprising the sequence of SEQ ID NO: 205, and a light chain variable region comprising the sequence of SEQ ID NO: 206. In some embodiments, the antibody or an antigen-binding fragment thereof does not compete for binding to human IL-36R with an antibody comprising a heavy chain variable region comprising the sequence of SEQ ID NO: 209, and a light chain variable region comprising the sequence of SEQ ID NO: 210.

In some embodiments, the anti-IL-36R antibody or an antigen-binding fragment thereof in this disclosure binds to human IL-36R with high specificity. In some embodiments, the anti-IL-36R antibody or an antigen-binding fragment thereof disclosed does not bind to cynomolgus IL-36R or mouse IL-36R. In some embodiments, the anti-IL-36R antibody or an antigen-binding fragment thereof disclosed does not bind to human IL-1R1.

In some embodiments, the antibody or an antigen-binding fragment thereof provided herein is bispecific. In some embodiments, the antibody or an antigen-binding fragment thereof provided herein is capable of specifically binding to a second antigen other than IL-36R, or a second epitope on IL-36R. In some embodiments, the second antigen other than IL-36R is selected from the group consisting of IL-17, IL-23, TNF, IL-12 and IL-1.

In another aspect, the present disclosure provides an antibody-drug conjugate, comprising an anti-IL-36R antibody or an antigen-binding fragment thereof disclosed herein, linked to one or more conjugate moieties. In some embodiments, the conjugate moiety can comprise a detectable marker, a drug, a toxin, a cytokine, a radionuclide, an enzyme, or any combinations thereof.

In some embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs.

In some embodiments, the conjugate moiety comprises a drug or a therapeutic agent.

In another aspect, the present disclosure provides a chimeric antigen receptor (CAR), comprising an antigen binding domain, a transmembrane domain, and a TCR signaling domain, wherein the antigen binding domain specifically binds to IL-36R and comprises an antigen binding fragment of the anti-IL-36R antibody provided herein. In some embodiments, the antigen binding fragment further comprises a costimulatory domain. In some embodiments, the antigen binding domain of the CAR specifically binds to human IL-36R and comprises an antigen binding fragment of the present disclosure.

In some embodiments, the antigen binding domain of the CAR specifically binds to human IL-36R and comprises a heavy chain variable region comprising HCDR1, HCDR2 and HCDR3 sequences as provided herein, and/or a light chain variable region comprising LCDR1, LCDR2 and LCDR3 sequences as provided herein. In some embodiments, the antigen binding domain of the CAR comprises the HCDR1 comprises an amino acid sequence of DYYX₁X₂(SEQ ID NO: 191), SEQ ID NO: 19, 33, 48, 64, 79, 94, 109, 124 or 139; the HCDR2 comprises an amino acid sequence of LIRNKAAGYTIYYX₃X₄X₅VKG (SEQ ID NO: 192), SEQ ID NO: 20, 34, 49, 65, 80, 95, 110, 125 or 140; and the HCDR3 comprises an amino acid sequence of SEQ ID NO: 5, 21, 35, 50, 66, 81, 96, 111, 126 or 141; wherein, X₁ is M or L; X₂ is N, H, S or R; X₃ is S or A; X₄ is A or D; X₅ is S or P, and/or the LCDR1 comprises an amino acid sequence of RASX₁₈NINIWLS (SEQ ID NO: 193), SEQ ID NO: 22, 36, 51, 67, 82, 97, 112, 127 or 142; the LCDR2 comprises an amino acid sequence of SEQ ID NO: 7, 23, 37, 52, 68, 83, 98, 113, 128 or 113; and the LCDR3 comprises an amino acid sequence of X₁₉QSQSYPLT (SEQ ID NO: 194), SEQ ID NO: 24, 38, 53, 69, 84, 99, 114, 129 or 143; wherein, X₁₈ is Q or R; X₁₉ is Q or L. In some embodiments, the HCDR1 comprises an amino acid sequence of SEQ ID NO: 3, 180, 184 or 188, the HCDR2 comprises an amino acid sequence of SEQ ID NO: 4, 151, 164 or 173, the HCDR3 comprises an amino acid sequence of SEQ ID NO: 5, the LCDR1 comprises an amino acid sequence of SEQ ID NO: 6 or 152, the LCDR2 comprises an amino acid sequence of SEQ ID NO: 7, and the LCDR3 comprises an amino acid sequence of SEQ ID NO: 8 or 153.

In some embodiments, the antigen binding domain of the CAR specifically binds to human IL-36R and comprises a pair of VH/VL selected from the group consisting of SEQ ID NOs: 1/2, 17/18, 31/32, 46/47, 62/63, 77/78, 92/93, 107/108, 122/123 and 137/138. In some embodiments, the antigen binding domain of the CAR specifically binds to human IL-36R and comprises a pair of VH/VL selected from the group consisting of SEQ ID NOs: 162/163, 171/172, 176/177, 179/150, 182/150, 149/150, 183/150, 185/150, 187/150 and 189/150.

In another aspect, the present disclosure provides a nucleic acid sequence encoding the chimeric antigen receptor (CAR) of the present application. In another aspect, the present disclosure provides a cell comprising the nucleic acid sequence of the present disclosure. In another aspect, the present disclosure provides a cell genetically modified to express the CAR of the present disclosure. In another aspect, the present disclosure provides a vector comprising the nucleic acid sequence of the present disclosure.

In another aspect, the present disclosure provides a method for stimulating a T cell-mediated immune response to an IL-36R-expressing cell or tissue in a mammal, the method comprising administering to the mammal an effective amount of a cell genetically modified to express the CAR of the present disclosure.

In another aspect, the present disclosure provides a method for treating a mammal having an IL-36R related disease or condition, comprising administering to the mammal an effective amount of a cell of the present disclosure, thereby treating the mammal. In some embodiments, the cell is an autologous T cell. In some embodiments, the mammal is a human subject. In some embodiments, the mammal is identified as having an IL-36R positive cell, or a cell with IL-36R signaling dys-regulated.

In another aspect, the present disclosure provides a pharmaceutical composition comprising the antibody or an antigen-binding fragment thereof, the polynucleotide encoding the antibody or antigen-binding fragment thereof, the antibody-drug conjugate, the CAR, or the recombinant immune cell of the present disclosure and one or more pharmaceutically acceptable carriers.

In another aspect, the present disclosure provides an isolated polynucleotide encoding the antibody or an antigen-binding fragment thereof of the present disclosure.

In another aspect, the present disclosure provides a vector comprising the isolated polynucleotide of the present disclosure.

In another aspect, the present disclosure provides a host cell comprising the vector of the present disclosure.

In another aspect, the present disclosure provides a kit comprising the antibody or an antigen-binding fragment thereof and/or the pharmaceutical composition of the present disclosure, and a second therapeutic agent.

In another aspect, the present disclosure provides a method of expressing the antibody or an antigen-binding fragment thereof of the present disclosure, comprising culturing the host cell of the present disclosure under the condition at which the vector of the present disclosure is expressed.

In another aspect, the present disclosure provides a method of treating, preventing or alleviating diseases or disorders responsive to IL-36R inhibition, comprising administering to the subject a therapeutically effective amount of the antibody or an antigen-binding fragment thereof, the polynucleotide encoding the antibody or antigen-binding fragment thereof, the antibody-drug conjugate, the recombinant immune cell, and/or the pharmaceutical composition of the present disclosure.

In some embodiments, the disease or disorder is associated with dysregulation of IL-36R mediated signaling. In some embodiments, the dysregulation of IL-36R mediated signaling includes dysregulation of IL-36 (e.g., IL-36α, IL-36β, or IL-36γ), IL-36R antagonist and/or IL-38. In some embodiments, the dysregulation of IL-36R mediated signaling includes over activation of IL-36 (e.g., IL-36α, IL-36β, or IL-36γ), or over suppression of IL-36R antagonist or over suppression of IL-38, compared with a control level (e.g., the level in a healthy subject).

In some embodiments, the disease or disorder is an inflammatory disease, an autoimmune disease, a respiratory disease, a metabolic disorder, or cancer.

In some embodiments, the inflammatory disease is selected from the group consisting of: allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, and other allergies. In some embodiments, the inflammatory disease is selected from the group consisting of allergic inflammation of the skin, lungs, and gastrointestinal tract, atopic dermatitis (also known as atopic eczema), asthma (allergic and non-allergic), and epithelial-mediated inflammation.

In some embodiments, the autoimmune disease is selected from the group consisting of:P multiple sclerosis, asthma, type 1 diabetes mellitus, rheumatoid arthritis, scleroderma, Crohn's disease, psoriasis vulgaris (commonly referred to as psoriasis), hidradenitis suppurativa, generalized pustular psoriasis (GPP), palmo-plantar pustulosis (PPP), inflammatory bowel disease, psoriatic arthritis, systemic lupus erythematosus (SLE), ulcerative colitis, ankylosing spondylitis atopic dermatitis and acne vulgaris. In some embodiments, the method is useful to treat pustular psoriasis, generalized pustular psoriasis, palmo-plantar pustulosis (PPP), psoriasis vulgaris, atopic dermatitis and Acne Vulgaris. In some embodiments, the autoimmune disease is selected from the group consisting of psoriasis vulgaris (commonly referred to as psoriasis), hidradenitis suppurativa, generalized pustular psoriasis (GPP), palmo-plantar pustulosis (PPP), inflammatory bowel disease, atopic dermatitis, acne vulgaris and Behcet's (beh-CHETS) disease (also called Behcet syndrome).

In some embodiments, the respiratory disease is selected from the group consisting of asthma, cystic fibrosis, emphysema, chronic obstructive pulmonary disease (COPD), and acute respiratory distress syndrome.

In some embodiments, the metabolic disease is selected from the group consisting of obesity, type 2 diabetes, atherosclerosis, and cardiovascular disease.

In some embodiments, the cancer can be any type of cancer known in the art, including but not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, leukemia, lymphoma, and Merkel cell carcinoma.

In some embodiments, the disease or disorder is selected from the group consisting of psoriasis, pustular psoriasis, hidradenitis suppurativa, ichthyosis, inflammatory bowel disease (IBD), atopic dermatitis, acne vulgaris, and Behcet's disease.

In another aspect, the present disclosure provides a method of modulating IL-36R activity in an IL-36R-positive cell, comprising exposing the IL-36R-positive cell to the antibody or antigen-binding fragment thereof of the present disclosure and/or the pharmaceutical composition of the present disclosure.

In another aspect, the present disclosure provides a method of detecting the presence or amount of IL-36R in a sample, comprising contacting the sample with the antibody or an antigen-binding fragment thereof of the present disclosure, and determining the presence or the amount of IL-36R in the sample.

In another aspect, the present disclosure provides a method of diagnosing an IL-36R related disease, disorder or condition in a subject, comprising: a) contacting a sample obtained from the subject with the antibody or an antigen-binding fragment thereof of the present disclosure and/or the pharmaceutical composition of the present disclosure; b) determining the presence or amount of IL-36R in the sample; and c) correlating the presence or the amount of IL-36R to existence or status of the IL-36R related disease, disorder or condition in the subject.

In certain embodiments, the antibody or an antigen-binding fragment thereof comprises the HCDR1 comprising the sequence of SEQ ID NO: 3, the HCDR2 comprising the sequence of SEQ ID NO: 151, the HCDR3 comprising the sequence of SEQ ID NO: 5, the LCDR1 comprising the sequence of SEQ ID NO: 152, the LCDR2 comprising the sequence of SEQ ID NO: 7, and the LCDR3 comprising the sequence of SEQ ID NO: 153.

In another aspect, the present disclosure provides use of the antibody or an antigen-binding fragment thereof of the present disclosure and/or the pharmaceutical composition of the present disclosure in the manufacture of a medicament for treating, preventing or alleviating diseases or disorders responsive to IL-36R inhibition in a subject.

In another aspect, the present disclosure provides use of the antibody or an antigen-binding fragment thereof of the present disclosure and/or the pharmaceutical composition of the present disclosure in the manufacture of a diagnostic reagent for diagnosing diseases or disorders responsive to IL-36R inhibition in a subject. In another aspect, the present disclosure provides a kit comprising the antibody or an antigen-binding fragment thereof of the present disclosure and/or the pharmaceutical composition of the present disclosure, useful in detecting IL-36R. In certain embodiments, the antibody or an antigen-binding fragment thereof comprises the HCDR1 comprising the sequence of SEQ ID NO: 3, the HCDR2 comprising the sequence of SEQ ID NO: 151, the HCDR3 comprising the sequence of SEQ ID NO: 5, the LCDR1 comprising the sequence of SEQ ID NO: 152, the LCDR2 comprising the sequence of SEQ ID NO: 7, and the LCDR3 comprising the sequence of SEQ ID NO: 153.

### BRIEF DESCRIPTION OF THE DRAWINGS

FIG. 1 shows binding affinity test of IL-36R antibodies.

FIG. 2 shows antibodies block IL-36R signaling (IL-36R reporter assay).

FIG. 3 shows blocking activity of humanized IL-36R antibody 5F7.

FIG. 4 shows blocking activity of 5F7 after affinity maturation.

FIG. 5 shows binding kinetics curve of antibodies to hIL-36R (tested by SPR).

FIG. 6 shows ELISA binding of 5F7-2a8 to hIL-36R-his.

FIG. 7 shows antibodies binding with membrane hIL-36R.

FIG. 8 shows antibodies binding with membrane cyno IL-36R.

FIG. 9 shows antibodies binding with membrane mouse IL-36R.

FIG. 10 shows binding test to hIL1R1.

FIG. 11 shows 5F7-2a8 block IL-36α/β/γ induced IL-8 release in A431 cells.

FIG. 12 shows 5F7-2a8 blocks IL-36α induced IL-8 release in HDF cells.

FIG. 13 shows 5F7-2a8 block IL-36α induced IL-8/IL-6/TNF-α release in HEK cells.

FIG. 14 shows 5F7-2a8 block IL-36β induced IL-8/IL-6/TNF-α release in HEK cells.

FIG. 15 shows 5F7-2a8 block IL-36γ induced IL-8/IL-6/TNF-α release in HEK cells.

FIG. 16 shows antibody binding affinity to hFcRn.

FIG. 17 shows pharmacokinetic study results of Fc variants of 5F7-2a8 in cynomolgus monkey.

FIG. 18 shows blocking activity of IL36R antibodies tested by IL36R reporter assay.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

“Single-chain Fv-Fc antibody” or “scFv-Fc” refers to an engineered antibody consisting of an scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 June 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another V_{H}-V_{L} moiety such that V_{H}'S of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate, and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two VH moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The term “affinity” as used herein refers to the strength of non-covalent interaction between an immunoglobulin molecule (i.e., antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to, surface plasmon resonance method, microscale thermophoresis method, HPLC-MS method and flow cytometry (such as FACS) method. A K_{D} value of ≤10⁻⁶ M (e.g. ≤5×10⁻⁷ M, ≤2×10⁻⁷ M, 10⁻⁷ M, ≤5×10 ⁻⁸ M, ≤2×10⁻⁸ M, ≤10⁻⁹ M, ≤5×10⁻⁹ M, ≤4×10⁻⁹M, ≤3×10⁻⁹M, ≤2×10⁻⁹ M, or ≤10⁻⁹ M) can indicate specific binding between an antibody or antigen binding fragments thereof and IL-36R (e.g. human IL-36R).

The ability to “compete for binding to human IL-36R” as used herein refers to the ability of a first antibody or antigen-binding fragment to inhibit the binding interaction between human IL-36R and a second anti-IL-36R antibody to any detectable degree. In certain embodiments, an antibody or antigen-binding fragment that compete for binding to human IL-36R inhibits the binding interaction between human IL-36R and a second anti-IL-36R antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Name of | Three-letter | Single-letter |
|---|---|---|
| Amino Acid | Code | Code |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g. Cys, Ser, Thr, Asn and Gln), among amino acid residues with acidic side chains (e.g. Asp, Glu), among amino acid residues with basic side chains (e.g. His, Lys, and Arg), or among amino acid residues with aromatic side chains (e.g. Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementary strand) or amino acid sequences that have sequence identity of at least 60% (e.g., at least 65%, 70%, 75%, 80%, 85%, 88%, 90%, 91%, 92%, 93%, 94%, 95%, 96%, 97%, 98%, 99%) to another sequence when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or an antigen-binding fragment thereof, at least one promoter (e.g., SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis” “diagnoses” or “diagnosing” refers to the identification of a pathological state, disease or condition, such as identification of an IL-36R related disease, or refer to identification of a subject with an IL-36R related disease who may benefit from a particular treatment regimen. In some embodiments, diagnosis contains the identification of abnormal amount or activity of IL-36R. In some embodiments, diagnosis refers to the identification of a cancer or an autoimmune disease in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject.

“IL-36R” as used herein, refers to interleukin-36 receptor, which is a cell surface receptor and a member of the IL1R family that is known to be involved in inflammatory responses triggered in skin and other epithelial tissues. IL-36R is a heterodimeric protein composed of a receptor subunit of IL-1Rrp2 (also known as IL-1RL2, Interleukin 1 receptor-like 2 or Interleukin 1 receptor-related protein 2) and a co-receptor subunit Interleukin-1 receptor, IL-1RAcP, an accessory protein shared with IL-1R and IL-33R. IL-36R recognizes three different agonists IL-36α, IL-36β and IL-36γ (also known as IL-1F6, IL-1F8, and IL-1F9), which signal through IL-36R/IL-1RAcP receptors to activate NF-κB and MAPK, such as p38 and JNK, and promote inflammatory response and induce the expression of inflammatory cytokines. There are also two receptor antagonists, IL-36Ra and IL-38, which bind to the IL-36 receptor and reduce the expression of inflammatory cytokines.

In certain embodiments, the IL-36R is human IL-36R, comprising human IL-1Rrp2 and human IL-1RAcP. In some embodiments, the human IL-1Rrp2 sequence is available under Genbank accession number NP_003845.2. In some embodiments, the amino acid sequence of human IL-1Rrp2 is as shown in SEQ ID NO: 211. In some embodiments, the human IL-1RAcP sequence is available under Genbank accession number NP_002173.1. In some embodiments, the amino acid sequence of human IL-1RAcP is as shown in SEQ ID NO: 212.

Amino acid sequence of human IL-1Rrp2 (SEQ ID
NO: 211):
MWSLLLCGLSIALPLSVTADGCKDIFMKNEILSASQPFAFNCTFPP
ITSGEVSVTWYKNSSKIPVSKIIQSRIHQDETWILFLPMEWGDSGVYQC
VIKGRDSCHRIHVNLTVFEKHWCDTSIGGLPNLSDEYKQILHLGKDDSL
TCHLHFPKSCVLGPIKWYKDCNEIKGERFTVLETRLLVSNVSAEDRGNY
ACQAILTHSGKQYEVLNGITVSITERAGYGGSVPKIIYPKNHSIEVQLG
TTLIVDCNVTDTKDNTNLRCWRVNNTLVDDYYDESKRIREGVETHVSFR
EHNLYTVNITFLEVKMEDYGLPFMCHAGVSTAYIILQLPAPDFRAYLIG
GLIALVAVAVSVVYIYNIFKIDIVLWYRSAFHSTETIVDGKLYDAYVLY
PKPHKESQRHAVDALVLNILPEVLERQCGYKLFIFGRDEFPGQAVANVI
DENVKLCRRLIVIVVPESLGFGLLKNLSEEQIAVYSALIQDGMKVILIE
LEKIEDYTVMPESIQYIKQKHGAIRWHGDFTEQSQCMKTKFWKTVRYHM
PPRRCRPFPPVQLLQHTPCYRTAGPELGSRRKKCTLTTG
Amino acid sequence of human IL-1RAcP (SEQ ID
NO: 212):
MTLLWCVVSLYFYGILQSDASERCDDWGLDTMRQIQVFEDEPA
RIKCPLFEHFLKFNYSTAHSAGLTLIWYWTRQDRDLEEPINFRLPENRI
SKEKDVLWFRPTLLNDTGNYTCMLRNTTYCSKVAFPLEVVQKDSCFNSP
MKLPVHKLYIEYGIQRITCPNVDGYFPSSVKPTITWYMGCYKIQNFNNV
IPEGMNLSFLIALISNNGNYTCVVTYPENGRTFHLTRTLTVKVVGSPKN
AVPPVIHSPNDHVVYEKEPGEELLIPCTVYFSFLMDSRNEVWWTIDGKK
PDDITIDVTINESISHSRTEDETRTQILSIKKVTSEDLKRSYVCHARSA
KGEVAKAAKVKQKVPAPRYTVELACGFGATVLLVVILIVVYHVYWLEMV
LFYRAHFGTDETILDGKEYDIYVSYARNAEEEEFVLLTLRGVLENEFGY
KLCIFDRDSLPGGIVTDETLSFIQKSRRLLVVLSPNYVLQGTQALLELK
AGLENMASRGNINVILVQYKAVKETKVKELKRAKTVLTVIKWKGEKSKY
PQGRFWKQLQVAMPVKKSPRRSSSDEQGLSYSSLKNV

The term “anti-IL-36R antibody” refers to an antibody that is capable of specifically binding to IL-36R (e.g., human IL-36R). The term “anti-human IL-36R antibody” refers to an antibody that is capable of specifically binding to human IL-36R.

A “IL-36R related” disease, disorder or condition as used herein refers to any disease or condition caused by, exacerbated by, or otherwise linked to increased or decreased expression or activities of IL-36R. In some embodiments, the IL-36R related disease, disorder or condition is an immune-related disorder, such as, for example, an autoimmune disease. In some embodiments, the IL-36R related disease, disorder or condition is a disorder related to excessive cell proliferation, such as, for example, cancer. In certain embodiments, the IL-36R related disease or condition is characterized in expressing or over-expressing of IL-36R gene. In certain embodiments, the IL-36R related disease or condition is characterized in over-expression of IL-36R and/or dysregulation of IL-36R mediated signaling.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “IL-36R-positive cell” as used herein refer to a cell which shows an abnormal expression level of IL-36R relative to a control cell. The abnormal expression level can be up-regulated or down-regulated relative to the level of the control cell, and can be associated with dysregulation of IL-36R mediated signaling.

The control cell can be a normal or healthy counterpart cell, which may or may not express IL-36R. In case the control cell expresses IL-36R, the abnormal expression level of the IL-36R-positive cell can be up-regulated or down-regulated. In case the control cell does not express IL-36R, the abnormal expression level of the IL-36R-positive cell can be up-regulated.

### Anti-IL-36R Antibodies

The present disclosure provides anti-IL-36R antibodies and antigen-binding fragments thereof. The anti-IL-36R antibodies and antigen-binding fragments provided herein are capable of specific binding to IL-36R.

In certain embodiments, the antibodies and the antigen-binding fragments thereof provided herein specifically bind to human IL-36R at an K_{D} value of no more than 10⁻⁷ M, no more than 8×10⁻⁸ M, no more than 5×10⁻⁸ M, no more than 2×10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, or no more than 6×10⁻¹⁰ M by Surface Plasmon Resonance (SPR) assay, see, for example, Murphy, M. et al., Current protocols in protein science, Chapter 19, unit 19.14, 2006. In certain embodiments, the K_{D} value is measured by the method as described in Example 6 of the present disclosure.

Binding of the antibodies or the antigen-binding fragments thereof provided herein to human IL-36R can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), flow cytometry assay, and other binding assays. In certain embodiments, the antibodies and the antigen-binding fragments thereof provided herein specifically bind to human IL-36R at an EC₅₀ (i.e., 50% binding concentration) of no more than 1 nM, no more than 0.9 nM, no more than 0.8 nM, no more than 0.7 nM, no more than 0.6 nM, no more than 0.5 nM, no more than 0.4 nM, no more than 0.3 nM, no more than 0.2 nM, no more than 0.1 nM, no more than 0.09 nM, no more than 0.08 nM, no more than 0.07 nM, no more than 0.06 nM or no more than 0.05 nM by enzyme linked immunosorbent assay (ELISA) assay. In certain embodiments, the antibodies and the antigen-binding fragments thereof provided herein specifically bind to human IL-36R at an EC₅₀ (i.e., 50% binding concentration) of no more than no more than 0.1 μg/mL, no more than 0.09 μg/mL, no more than 0.08 μg/mL, no more than 0.07 μg/mL, no more than 0.06 μg/mL, no more than 0.05 μg/mL, no more than 0.04 μg/mL, no more than 0.03 μg/mL, no more than 0.02 μg/mL, no more than 0.01 μg/mL, or no more than 0.005 μg/mL by ELISA assay. In certain embodiments, the EC₅₀ value is measured by the method as described in Example 7 of the present disclosure.

In certain embodiments, the antibodies and antigen-binding fragments thereof provided herein specifically bind to membrane human IL-36R as measured by Fluorescence activated Cell Sorting (FACS) assay. In certain embodiments, the antibodies and antigen-binding fragments thereof provided herein specifically bind to membrane human IL-36R at a binding affinity with an EC₅₀ of no more than 0.5 g/mL, no more than 0.4 μg/mL, no more than 0.3 μg/mL, no more than 0.2 μg/mL, no more than 0.19 μg/mL, no more than 0.18 μg/mL, no more than 0.17 μg/mL, no more than 0.16 μg/mL, no more than 0.15 μg/mL, or no more than 0.1 μg/mL as measured by FACS assay. In certain embodiments, the EC₅₀ value is measured by the method as described in Example 8 of the present disclosure.

In certain embodiments, the antibodies and antigen-binding fragments thereof provided herein specifically bind to human IL-36R as measured by Biolayer interferometry (BLI). In certain embodiments, the antibodies and antigen-binding fragments thereof provided herein having a binding affinity to recombinant human IL-36R protein with a K_{d} of no more than 2E-08 M (preferably no more than 1E-08 M, for example, no more than 9E-09M, no more than 8E-09M, no more than 7E-09M, no more than 6E-09M, no more than 5E-09M, no more than 4E-09M, no more than 3E-09M, no more than 2E-09M, or no more than 1E-10 M) as measured by BLI. In certain embodiments, the K_{d} value is measured by the method as described in Example 2 of the present disclosure.

In certain embodiments, the antibodies and antigen-binding fragments thereof provided herein exhibit no detectable binding to cynomolgus or mouse IL-36R or exhibits a binding to cynomolgus or mouse IL-36R at a level comparable to that of a negative control antibody under equivalent assay conditions. In addition, a negative control antibody can be any antibody that is known not to bind to cynomolgus or mouse IL-36R.

In certain embodiments, the antibodies and antigen-binding fragments thereof provided herein is capable of blocking IL-36R signaling induced by an IL-36R agonist (such as IL-36α, IL-36β, and/or IL-36γ, preferably IL-36α), as measured by IL-36R reporter assay. In certain embodiments, the IL-36R reporter assay is as described in Example 3 of the present disclosure.

In some embodiments, the antibody or antigen-binding fragment thereof of the present disclosure has the ability of inhibiting IL-36R agonist-induced IL-6 release in a cell, wherein the IL-36R agonist includes IL-36α, IL-36β, and/or IL-36γ. In some embodiments, the antibody or antigen-binding fragment thereof of the present disclosure has the ability of inhibiting IL-36R agonist-induced TNF-α release in a cell, wherein the IL-36R agonist includes IL-36α, IL-36β, and/or IL-36γ.

### Illustrative Anti-IL-36R Antibodies

In certain embodiments, the present disclosure provides anti-IL-36R antibodies (e.g., anti-human IL-36R antibodies) and antigen-binding fragments thereof comprising one or more (e.g., 1, 2, or 3) HCDRs comprising the sequences selected from the group consisting of DYYX₁X₂ (SEQ ID NO: 191), LIRNKAAGYTIYYX₃X₄X₅VKG (SEQ ID NO: 192), SEQ ID NOs: 19, 33, 48, 64, 79, 94, 109, 124, 139, 20, 34, 49, 65, 80, 95, 110, 125, 140, 5, 21, 35, 50, 66, 81, 96, 111, 126 and 141, wherein X₁ is M or L; X₂ is N, H, S or R; X₃ is S or A; X₄ is A or D; X₅ is S or P. In certain embodiments, the present disclosure further encompasses antibodies and antigen binding fragments thereof having no more than one, two or three amino acid residue substitutions to any of the sequences herein.

In certain embodiments, the present disclosure provides anti-IL-36R antibodies (e.g., anti-human IL-36R antibodies) and antigen-binding fragments thereof comprising one or more (e.g., 1, 2, or 3) LCDRs comprising the sequences selected from the group consisting of RASX₁₈NINIWLS (SEQ ID NO: 193), X₁₉QSQSYPLT (SEQ ID NO: 194), SEQ ID NOs: 22, 36, 51, 67, 82, 97, 112, 127, 142, 7, 23, 37, 52, 68, 83, 98, 113, 128, 113, 24, 38, 53, 69, 84, 99, 114, 129 and 143, wherein X₁₈ is Q or R; X₁₉ is Q or L. In certain embodiments, the present disclosure further encompasses antibodies and antigen binding fragments thereof having no more than one, two or three amino acid residue substitutions to any of the sequences herein.

Antibody “5F7” as used herein refers to a monoclonal antibody comprising a heavy chain variable region having the sequence of SEQ ID NO: 1, and a light chain variable region having the sequence of SEQ ID NO: 2.

Antibody “9A6” as used herein refers to a monoclonal antibody comprising a heavy chain variable region having the sequence of SEQ ID NO: 17, and a light chain variable region having the sequence of SEQ ID NO: 18.

Antibody “9C12” as used herein refers to a monoclonal antibody comprising a heavy chain variable region having the sequence of SEQ ID NO: 31, and a light chain variable region having the sequence of SEQ ID NO: 32.

Antibody “10D12” as used herein refers to a monoclonal antibody comprising a heavy chain variable region having the sequence of SEQ ID NO: 46, and a light chain variable region having the sequence of SEQ ID NO: 47.

Antibody “10F6” as used herein refers to a monoclonal antibody comprising a heavy chain variable region having the sequence of SEQ ID NO: 62, and a light chain variable region having the sequence of SEQ ID NO: 63.

Antibody “9H11” as used herein refers to a monoclonal antibody comprising a heavy chain variable region having the sequence of SEQ ID NO: 77, and a light chain variable region having the sequence of SEQ ID NO: 78.

Antibody “1C11” as used herein refers to a monoclonal antibody comprising a heavy chain variable region having the sequence of SEQ ID NO: 92, and a light chain variable region having the sequence of SEQ ID NO: 93.

Antibody “1A21” as used herein refers to a monoclonal antibody comprising a heavy chain variable region having the sequence of SEQ ID NO: 107, and a light chain variable region having the sequence of SEQ ID NO: 108.

Antibody “1J3” as used herein refers to a monoclonal antibody comprising a heavy chain variable region having the sequence of SEQ ID NO: 122, and a light chain variable region having the sequence of SEQ ID NO: 123.

Antibody “1J22” as used herein refers to a monoclonal antibody comprising a heavy chain variable region having the sequence of SEQ ID NO: 137, and a light chain variable region having the sequence of SEQ ID NO: 138.

In certain embodiments, the present disclosure provides anti-IL-36R antibodies and antigen-binding fragments thereof comprising one or more (e.g., 1, 2, 3, 4, 5, or 6) CDRs sequences of Antibody 5F7, 9A6, 9C12, 10D12, 10F6, 9H11, 1C11, 1A21, 1J3 or 1J22.

In certain embodiments, the present disclosure provides anti-IL-36R antibodies and antigen-binding fragments thereof comprising HCDR1 comprising the sequence selected from the group consisting of SEQ ID NOs: 3, 19, 33, 48, 64, 79, 94, 109, 124 and 139, HCDR2 comprising the sequence selected from the group consisting of SEQ ID NOs: 4, 20, 34, 49, 65, 80, 95, 110, 125 and 140, and HCDR3 comprising the sequence selected from the group consisting of SEQ ID NOs: 5, 21, 35, 50, 66, 81, 96, 111, 126 and 141, and/or LCDR1 comprising the sequence selected from the group consisting of SEQ ID NOs: 6, 22, 36, 51, 67, 82, 97, 112, 127 and 142, LCDR2 comprising the sequence selected from the group consisting of SEQ ID NOs: 7, 23, 37, 52, 68, 83, 98, 113, and 128, and LCDR3 comprising the sequence selected from the group consisting of SEQ ID NOs: 8, 24, 38, 53, 69, 84, 99, 114, 129 and 143.

In certain embodiments, the present disclosure provides anti-IL-36R antibodies and antigen-binding fragments thereof comprising a HCDR1 comprising the sequence of SEQ ID NO: 3, a HCDR2 comprising the sequence of SEQ ID NO: 4, a HCDR3 comprising the sequence of SEQ ID NO: 5, and/or a LCDR1 comprising the sequence of SEQ ID NO: 6, a LCDR2 comprising the sequence of SEQ ID NO: 7, and a LCDR3 comprising the sequence of SEQ ID NO: 8.

In certain embodiments, the present disclosure provides anti-IL-36R antibodies and antigen-binding fragments thereof comprising a HCDR1 comprising the sequence of SEQ ID NO: 19, a HCDR2 comprising the sequence of SEQ ID NO: 20, a HCDR3 comprising the sequence of SEQ ID NO: 21, and/or a LCDR1 comprising the sequence of SEQ ID NO: 22, a LCDR2 comprising the sequence of SEQ ID NO: 23, and a LCDR3 comprising the sequence of SEQ ID NO: 24.

In certain embodiments, the present disclosure provides anti-IL-36R antibodies and antigen-binding fragments thereof comprising a HCDR1 comprising the sequence of SEQ ID NO: 33, a HCDR2 comprising the sequence of SEQ ID NO: 34, a HCDR3 comprising the sequence of SEQ ID NO: 35, and/or a LCDR1 comprising the sequence of SEQ ID NO: 36, a LCDR2 comprising the sequence of SEQ ID NO: 37, and a LCDR3 comprising the sequence of SEQ ID NO: 38.

In certain embodiments, the present disclosure provides anti-IL-36R antibodies and antigen-binding fragments thereof comprising a HCDR1 comprising the sequence of SEQ ID NO: 48, a HCDR2 comprising the sequence of SEQ ID NO: 49, a HCDR3 comprising the sequence of SEQ ID NO: 50, and/or a LCDR1 comprising the sequence of SEQ ID NO: 51, a LCDR2 comprising the sequence of SEQ ID NO: 52, and a LCDR3 comprising the sequence of SEQ ID NO: 53.

In certain embodiments, the present disclosure provides anti-IL-36R antibodies and antigen-binding fragments thereof comprising a HCDR1 comprising the sequence of SEQ ID NO: 64, a HCDR2 comprising the sequence of SEQ ID NO: 65, a HCDR3 comprising the sequence of SEQ ID NO: 66, and/or a LCDR1 comprising the sequence of SEQ ID NO: 67, a LCDR2 comprising the sequence of SEQ ID NO: 68, and a LCDR3 comprising the sequence of SEQ ID NO: 69.

In certain embodiments, the present disclosure provides anti-IL-36R antibodies and antigen-binding fragments thereof comprising a HCDR1 comprising the sequence of SEQ ID NO: 79, a HCDR2 comprising the sequence of SEQ ID NO: 80, a HCDR3 comprising the sequence of SEQ ID NO: 81, and/or a LCDR1 comprising the sequence of SEQ ID NO: 82, a LCDR2 comprising the sequence of SEQ ID NO: 83, and a LCDR3 comprising the sequence of SEQ ID NO: 84.

In certain embodiments, the present disclosure provides anti-IL-36R antibodies and antigen-binding fragments thereof comprising a HCDR1 comprising the sequence of SEQ ID NO: 94, a HCDR2 comprising the sequence of SEQ ID NO: 95, a HCDR3 comprising the sequence of SEQ ID NO: 96, and/or a LCDR1 comprising the sequence of SEQ ID NO: 97, a LCDR2 comprising the sequence of SEQ ID NO: 98, and a LCDR3 comprising the sequence of SEQ ID NO: 99.

In certain embodiments, the present disclosure provides anti-IL-36R antibodies and antigen-binding fragments thereof comprising a HCDR1 comprising the sequence of SEQ ID NO: 109, a HCDR2 comprising the sequence of SEQ ID NO: 110, a HCDR3 comprising the sequence of SEQ ID NO: 111, and/or a LCDR1 comprising the sequence of SEQ ID NO: 112, a LCDR2 comprising the sequence of SEQ ID NO: 113, and a LCDR3 comprising the sequence of SEQ ID NO: 114.

In certain embodiments, the present disclosure provides anti-IL-36R antibodies and antigen-binding fragments thereof comprising a HCDR1 comprising the sequence of SEQ ID NO: 124, a HCDR2 comprising the sequence of SEQ ID NO: 125, a HCDR3 comprising the sequence of SEQ ID NO: 126, and/or a LCDR1 comprising the sequence of SEQ ID NO: 127, a LCDR2 comprising the sequence of SEQ ID NO: 128, and a LCDR3 comprising the sequence of SEQ ID NO: 129.

In certain embodiments, the present disclosure provides anti-IL-36R antibodies and antigen-binding fragments thereof comprising a HCDR1 comprising the sequence of SEQ ID NO: 139, a HCDR2 comprising the sequence of SEQ ID NO: 140, a HCDR3 comprising the sequence of SEQ ID NO: 141, and/or a LCDR1 comprising the sequence of SEQ ID NO: 142, a LCDR2 comprising the sequence of SEQ ID NO: 113, and a LCDR3 comprising the sequence of SEQ ID NO: 143.

Table 1 below shows the CDR amino acid sequences of antibodies 5F7, 9A6, 9C12, 101D12, 10F6, 9H11, 1C11, 1A21, 1J3 and 1J22. The CDR boundaries were defined or identified by the convention of Kabat. Table 2 below shows the heavy chain and light chain variable region amino acid sequences of antibodies 5F7, 9A6, 9C12, 101S12, 10F6, 9H11, 2C2Q, 1A21, 1J3 and 1J22.

TABLE 1
CDR amino acid sequences of 10 antibodies

| Antibody | | CDR1 | CDR2 | CDR3 |
|---|---|---|---|---|
| 5F7 | HCDR | SEQ ID NO: 3 | SEQ ID NO: 4 | SEQ ID NO: 5 |
| | | DYYMN | LIRNKAAGYTI | DMTIYDGYYV |
| | | | YYSASVKG | DYALDY |
| | LCDR | SEQ ID NO: 6 | SEQ ID NO: 7 | SEQ ID NO: 8 |
| | | RASQNINIWLS | KASNLHT | QQSQSYPLT |
| 9A6 | HCDR | SEQ ID NO: 19 | SEQ ID NO: 20 | SEQ ID NO: 21 |
| | | SYDVS | VIWTGGGTDY | VYYDPYYAMD |
| | | | NSAFMS | Y |
| | LCDR | SEQ ID NO: 22 | SEQ ID NO: 23 | SEQ ID NO: 24 |
| | | SASSSVSSRYL | GTSNLAS | QQYHSDPLT |
| | | H | | |
| 9C12 | HCDR | SEQ ID NO: 33 | SEQ ID NO: 34 | SEQ ID NO: 35 |
| | | DYHMN | RINPDYGDARY | DGYYVFDY |
| | | | DQKFKG | |
| | LCDR | SEQ ID NO: 36 | SEQ ID NO: 37 | SEQ ID NO: 38 |
| | | RSSKSLLHSDG | RMSNLVS | AQTLEFPLT |
| | | ITYLS | | |
| 10D12 | HCDR | SEQ ID NO: 48 | SEQ ID NO: 49 | SEQ ID NO: 50 |
| | | RYWMH | NINPNIGRANY | PGETTDY |
| | | | NEKFKN | |
| | LCDR | SEQ ID NO: 51 | SEQ ID NO: 52 | SEQ ID NO: 53 |
| | | KSSQSLLYTDG | LVSKLDS | LQSTHYPFT |
| | | KTYLN | | |
| 10F6 | HCDR | SEQ ID NO: 64 | SEQ ID NO: 65 | SEQ ID NO: 66 |
| | | EFTIH | WFYPGSDFIKY | HEDRLSDYSYE |
| | | | NEKFKD | WYFDV |
| | LCDR | SEQ ID NO: 67 | SEQ ID NO: 68 | SEQ ID NO: 69 |
| | | RASENIDSYLA | GATLLAD | QHYYSIPLT |
| 9H11 | HCDR | SEQ ID NO: 79 | SEQ ID NO: 80 | SEQ ID NO: 81 |
| | | HSWMN | WIHLGDGDTY | SDGNYVPYAM |
| | | | YNGKFKG | DY |
| | LCDR | SEQ ID NO: 82 | SEQ ID NO: 83 | SEQ ID NO: 84 |
| | | RASQDISNYLS | YTSRLHS | QQDSEHPWT |
| 1C11 | HCDR | SEQ ID NO: 94 | SEQ ID NO: 95 | SEQ ID NO: 96 |
| | | NYWLH | NINPSNGGTNY | YSTGFAY |
| | | | NEKFKT | |
| | LCDR | SEQ ID NO: 97 | SEQ ID NO: 98 | SEQ ID NO: 99 |
| | | RASESVDTYG | RASNLES | QQSNEDPRT |
| | | ASFMH | | |
| 1A21 | HCDR | SEQ ID NO: 109 | SEQ ID NO: 110 | SEQ ID NO: 111 |
| | | GYFMN | RINPYNGDTFY | LGHWYFDV |
| | | | NQKFKG | |
| | LCDR | SEQ ID NO: 112 | SEQ ID NO: 113 | SEQ ID NO: 114 |
| | | RSSQSIVHSNG | KVSNRFS | FQGSHVPWT |
| | | NTYLE | | |
| 1J3 | HCDR | SEQ ID NO: 124 | SEQ ID NO: 125 | SEQ ID NO: 126 |
| | | SYGMS | TISSYGSYTSYP | PDYYVSTGFDY |
| | | | DTVKG | |
| | LCDR | SEQ ID NO: 127 | SEQ ID NO: 128 | SEQ ID NO: 129 |
| | | RASQSISDYLH | YASQSIS | QNGHSFPFT |
| 1J22 | HCDR | SEQ ID NO: 139 | SEQ ID NO: 140 | SEQ ID NO: 141 |
| | | DYTIH | WFYPGSGGLK | HGNYDGFAY |
| | | | YHEKFKD | |
| | LCDR | SEQ ID NO: 142 | SEQ ID NO: 113 | SEQ ID NO: 143 |
| | | RSSQSLVHSNG | KVSNRFS | SQSTHVPFT |
| | | NTYLH | | |

TABLE 2
Variable region amino acid sequences of 10 antibodies

| Antibody | VH | VL |
|---|---|---|
| 5F7 | SEQ ID NO: 1 | SEQ ID NO: 2 |
| | EVKLVESGGGLVQPGGSLSLSC | DIQMNQSPSSLSASLGDTITI |
| | AASGFAFTDYYMNWVRQPPG | TCRASQNINIWLSWYQQKPG |
| | KALEWLALIRNKAAGYTIYYS | NIPKLFIYKASNLHTGVPSRF |
| | ASVKGRFTISRDNSQSILYLQM | SGSGSGTDFTLTISSLQPEDIA |
| | TALRPEDSATYYCVRDMTIYDG | TYYCQQSQSYPLTFGAGTKL |
| | YYVDYALDYWGQGTSVTVSS | ELK |
| 9A6 | SEQ ID NO: 17 | SEQ ID NO: 18 |
| | QVQLQQSGPGLMAPSQSLSITC | DIVLTQSPAIMSASPGEKVTM |
| | TVSGFSLTSYDVSWIRQSPGKG | TCSASSSVSSRYLHWYQQKS |
| | LEWLGVIWTGGGTDYNSAFMS | GASPKLWIYGTSNLASGVPA |
| | RLSITKDNSKSQVFLKMSSLQT | RFSGSGSGTSYSLTISSVEAE |
| | DDTAIYYCVRVYYDPYYAMDY | DAATYYCQQYHSDPLTFGA |
| | WGQGTSVTVSS | GTKLELK |
| 9C12 | SEQ ID NO: 31 | SEQ ID NO: 32 |
| | EVQLQQSGPELVKPGASVKMS | DIVMTQAAFSNPVSLGTSASI |
| | CKASGYTFTDYHMNWVKRSP | SCRSSKSLLHSDGITYLSWY |
| | GKSLEWIGRINPDYGDARYDQ | LQRPGQSPQLLIYRMSNLVS |
| | KFKGKATLTVDKSLSTAYMQL | GVPDRFSGSGSGTDFTLRISR |
| | NRLTYEDSAVYYCARDGYYVF | VEAEDVGVYYCAQTLEFPLT |
| | DYWGQGSTLTVSS | FGAGTKLELK |
| 10D12 | SEQ ID NO: 46 | SEQ ID NO: 47 |
| | QVQLQQPGTELVKPGASVKLS | DVVMTQTPLILSVTIGQPASI |
| | CKASGYTFTRYWMHWVRQRP | SCKSSQSLLYTDGKTYLNWL |
| | GQGLEWIGNINPNIGRANYNEK | LQRPGQSPKRLIYLVSKLDSG |
| | FKNKATLTVDKSSSTAYMQLSS | VPDRFSGSGSGTDFTLKISRV |
| | LTSEDSAVYYCARPGETTDYW | EAVDLGVYYCLQSTHYPFTF |
| | GQGTTLTVSS | GTGTKLEIK |
| 10F6 | SEQ ID NO: 62 | SEQ ID NO: 63 |
| | QVQLQQSGAELVKPGASVRLS | DIQMTQSPASLSASVGETVTI |
| | CKASGYTFTEFTIHWVKQRSG | TCRASENIDSYLAWYQQKQ |
| | QGLEWIGWFYPGSDFIKYNEKF | GKSPQLLVYGATLLADGVPS |
| | KDKATLTADKSSNTVYMELLR | RFSGSGSGTQYSLKINSLQSE |
| | LTSEDSAVYFCARHEDRLSDYS | DVAEYFCQHYYSIPLTFGAG |
| | YEWYFDVWGAGTAVTVSS | TKLELK |
| 9H11 | SEQ ID NO: 77 | SEQ ID NO: 78 |
| | QVQLQQSGPELVKPGASVKISC | DIVITQTTSSLSASLGDRVTIS |
| | KASGYAFSHSWMNWVKQRPG | CRASQDISNYLSWYQQKPD |
| | KGLEWIGWIHLGDGDTYYNGK | GTVKLLIYYTSRLHSGVPSRF |
| | FKGKATLTADKSSSTAYMQLSS | SGSGSGTDYSLTISSLEQEDI |
| | LTSEDSAVYFCARSDGNYVPYA | ATYFCQQDSEHPWTFGGGTE |
| | MDYWGQGTSVTVSS | LDVR |
| 1C11 | SEQ ID NO: 92 | SEQ ID NO: 93 |
| | EVQVQQSGTELVKPGASVKLS | DIVLTQTTASLAVSLGQRATI |
| | CKASGYTFTNYWLHWVKQRP | SCRASESVDTYGASFMHWY |
| | GQGLEWIGNINPSNGGTNYNE | QQKPGQPPKLLIYRASNLES |
| | KFKTKATLTVDKSSSTAYMQLS | GIPARFSGSGSRTDFTLTVNP |
| | SLTSEDSAVYYCARYSTGFAYW | VEADDVATYYCQQSNEDPRT |
| | GQGTLVTVSA | FGGGTKLEIK |
| 1A21 | SEQ ID NO: 107 | SEQ ID NO: 108 |
| | EVQLQQSGPELVKPGDSVKISC | DIVMTQSTLSLPVSLGDQASI |
| | KASGYSFTGYFMNWVMQSHG | SCRSSQSIVHSNGNTYLEWY |
| | KSLEWIGRINPYNGDTFYNQKF | LQKPGQSPKLLIYKVSNRFS |
| | KGKATLTVDKSSSTAHMELRSL | GVPDRFSGSGSGTDFTLKISR |
| | TSEDSAVYYCAGLGHWYFDV | VEAEDLGVYYCFQGSHVPW |
| | WGTGTTVTVSS | TFGGGTKLEIK |
| 1J3 | SEQ ID NO: 122 | SEQ ID NO: 123 |
| | QVQLEQSGGDLVKPGGSLKLS | DIVITQSTATLSVTPGDRVSLS |
| | CAASGFTFSSYGMSWVRQTPD | CRASQSISDYLHWYQQKSHE |
| | KRLEWVATISSYGSYTSYPDTV | SPRLLIKYASQSISGIPSRFSG |
| | KGRFTISRDNAKNTLYLQMSSL | SGSGSDFTLSINSVEPEDVGV |
| | KSEDTAMYYCARPDYYVSTGF | YYCQNGHSFPFTFGSGTKLEI |
| | DYWGQGTTLTVSS | K |
| 1J22 | SEQ ID NO: 137 | SEQ ID NO: 138 |
| | QVQAQQSGAVLVQPGASVKLS | DIVLTQTPLSLPVSLGDQASI |
| | CKASGYTFTDYTIHWIKQRSGQ | SYRSSQSLVHSNGNTYLHW |
| | GLEWIGWFYPGSGGLKYHEKF | YLQKPGQSPKLLIYKVSNRF |
| | KDKATLTADKSSSTVYMELSRL | SGVPDRFSGSGSGTDFTLKIS |
| | TSEDSAVYFCTRHGNYDGFAY | RVEAEDLGVYFCSQSTHVPF |
| | WGQGTLVTVSA | TFGSGTKLEIK |

Given that each of antibodies 5F7, 9A6, 9C12, 10D12, 10F6, 9H11, 1C11, 1A21, 1J3 and 1J22 can bind to IL-36R and that antigen-binding specificity is provided primarily by the CDR1, CDR2 and CDR3 regions, the HCDR1, HCDR2 and HCDR3 sequences and LCDR1, LCDR2 and LCDR3 sequences of antibodies 5F7, 9A6, 9C12, 101D12, 10F6, 9H11, 1C11, 1A21, 1J3 and 1J22 can be “mixed and matched” (i.e., CDRs from different antibodies can be mixed and matched, but each antibody must contain a HCDR1, HCDR2 and HCDR3 and a LCDR1, LCDR2 and LCDR3) to create anti-IL-36R binding molecules of the present disclosure. IL-36R binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence (s). Likewise, when VL CDR sequences are mixed and matched, the LCDR1, LCDR2 and/or LCDR3 sequence from a particular VL sequence preferably is replaced with a structurally similar CDR sequence (s). It will be readily apparent to a person skilled in the art that novel VH and VL sequences can be created by substituting one or more VH and/or VL CDR region sequences with structurally similar sequences from the CDR sequences disclosed herein for monoclonal antibodies 5F7, 9A6, 9C12, 10D12, 10F6, 9H11, 1C11, 1A21, 1J3 and 1J22.

CDRs are known to be responsible for antigen binding. However, it has been found that not all of the 6 CDRs are indispensable or unchangeable. In other words, it is possible to replace or change or modify one or more CDRs in anti-IL-36R antibodies 5F7, 9A6, 9C12, 10D12, 10F6, 9H11, 1C11, 1A21, 1J3 and 1J22, yet substantially retain the specific binding affinity to IL-36R.

In certain embodiments, the antibodies and antigen-binding fragments thereof provided herein comprise suitable framework region (FR) sequences, as long as the antibodies and antigen-binding fragments thereof can specifically bind to IL-36R. The CDR sequences provided in Table 1 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al. (1987) J. Mol. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

Table 3 below shows the CDR amino acid sequences of humanized antibodies for antibody 5F7, which are designated as 5F7-hu-2, 5F7-hu-3, 5F7-hu-5, 5F7-2a1, 5F7-2a6, 5F7-2a8, 5F7-2c4, 5F7-2d3, 5F7-2g10 and 5F7-2h1. The CDR boundaries were defined or identified by the convention of Kabat. Table 4 below shows the heavy chain and light chain variable region amino acid sequences of humanized antibodies 5F7-hu-2, 5F7-hu-3, 5F7-hu-5, 5F7-2a1, 5F7-2a6, 5F7-2a8, 5F7-2c4, 5F7-2d3, 5F7-2g10 and 5F7-2h1. Table 5 below shows the FR amino acid sequences of 8 humanized antibodies 5F7-hu-2, 5F7-hu-3, 5F7-hu-5, 5F7-2a1, 5F7-2a6, 5F7-2a8, 5F7-2c4, 5F7-2d3, 5F7-2g10 and 5F7-2h1.

TABLE 3
CDR amino acid sequences of 10 humanized antibodies

| Antibody | | CDR1 | CDR2 | CDR3 |
|---|---|---|---|---|
| 5F7-hu-2 | HCDR | SEQ ID NO: 3 | SEQ ID NO: 164 | SEQ ID NO: 5 |
| | | DYYMN | LIRNKAAGYTI | DMTIYDGYYVD |
| | | | YYADSVKG | YALDY |
| | LCDR | SEQ ID NO: 6 | SEQ ID NO: 7 | SEQ ID NO: 153 |
| | | RASQNINIWLS | KASNLHT | LQSQSYPLT |
| 5F7-hu-3 | HCDR | SEQ ID NO: 3 | SEQ ID NO: 173 | SEQ ID NO: 5 |
| | | DYYMN | LIRNKAAGYTI | DMTIYDGYYVD |
| | | | YYSDSVKG | YALDY |
| | LCDR | SEQ ID NO: 6 | SEQ ID NO: 7 | SEQ ID NO: 153 |
| | | RASQNINIWLS | KASNLHT | LQSQSYPLT |
| 5F7-hu-5 | HCDR | SEQ ID NO: 3 | SEQ ID NO: 151 | SEQ ID NO: 5 |
| | | DYYMN | LIRNKAAGYTI | DMTIYDGYYVD |
| | | | YYSAPVKG | YALDY |
| | LCDR | SEQ ID NO: 6 | SEQ ID NO: 7 | SEQ ID NO: 153 |
| | | RASQNINIWLS | KASNLHT | LQSQSYPLT |
| 5F7-2a1 | HCDR | SEQ ID NO: 180 | SEQ ID NO: 151 | SEQ ID NO: 5 |
| | | DYYMH | LIRNKAAGYTI | DMTIYDGYYVD |
| | | | YYSAPVKG | YALDY |
| | LCDR | SEQ ID NO: 152 | SEQ ID NO: 7 | SEQ ID NO: 153 |
| | | RASRNINIWLS | KASNLHT | LQSQSYPLT |
| 5F7-2a6 | HCDR | SEQ ID NO: 3 | SEQ ID NO: 151 | SEQ ID NO: 5 |
| | | DYYMN | LIRNKAAGYTI | DMTIYDGYYVD |
| | | | YYSAPVKG | YALDY |
| | LCDR | SEQ ID NO: 152 | SEQ ID NO: 7 | SEQ ID NO: 153 |
| | | RASRNINIWLS | KASNLHT | LQSQSYPLT |
| 5F7-2a8 | HCDR | SEQ ID NO: 3 | SEQ ID NO: 151 | SEQ ID NO: 5 |
| | | DYYMN | LIRNKAAGYTI | DMTIYDGYYVD |
| | | | YYSAPVKG | YALDY |
| | LCDR | SEQ ID NO: 152 | SEQ ID NO: 7 | SEQ ID NO: 153 |
| | | RASRNINIWLS | KASNLHT | LQSQSYPLT |
| 5F7-2c4 | HCDR | SEQ ID NO: 184 | SEQ ID NO: 151 | SEQ ID NO: 5 |
| | | DYYLR | LIRNKAAGYTI | DMTIYDGYYVD |
| | | | YYSAPVKG | YALDY |
| | LCDR | SEQ ID NO: 152 | SEQ ID NO: 7 | SEQ ID NO: 153 |
| | | RASRNINIWLS | KASNLHT | LQSQSYPLT |
| 5F7-2d3 | HCDR | SEQ ID NO: 3 | SEQ ID NO: 151 | SEQ ID NO: 5 |
| | | DYYMN | LIRNKAAGYTI | DMTIYDGYYVD |
| | | | YYSAPVKG | YALDY |
| | LCDR | SEQ ID NO: 152 | SEQ ID NO: 7 | SEQ ID NO: 153 |
| | | RASRNINIWLS | KASNLHT | LQSQSYPLT |
| 5F7-2g10 | HCDR | SEQ ID NO: 188 | SEQ ID NO: 151 | SEQ ID NO: 5 |
| | | DYYLS | LIRNKAAGYTI | DMTIYDGYYVD |
| | | | YYSAPVKG | YALDY |
| | LCDR | SEQ ID NO: 152 | SEQ ID NO: 7 | SEQ ID NO: 153 |
| | | RASRNINIWLS | KASNLHT | LQSQSYPLT |
| 5F7-2h1 | HCDR | SEQ ID NO: 3 | SEQ ID NO: 151 | SEQ ID NO: 5 |
| | | DYYMN | LIRNKAAGYTI | DMTIYDGYYVD |
| | | | YYSAPVKG | YALDY |
| | LCDR | SEQ ID NO: 152 | SEQ ID NO: 7 | SEQ ID NO: 153 |
| | | RASRNINIWLS | KASNLHT | LQSQSYPLT |

TABLE 4
Variable region amino acid sequences of 10 humanized
antibodies

| Antibody | VH | VL |
|---|---|---|
| 5F7-hu-2 | SEQ ID NO: 162 | SEQ ID NO: 163 |
| | QVQLQESGGGLVKPGGSLRL | EIVMTQSPATLSVSPGERATL |
| | SCAASGFAFTDYYMNWIRQA | SCRASQNINIWLSWYQQKPG |
| | PGKGLEWVSLIRNKAAGYTIY | QAPRLFIYKASNLHTGVPAR |
| | YADSVKGRFTISRDNAKSSLY | FSGSGSGTEFTLTISSLQSEDF |
| | LQMNSLRAEDTAVYYCVRD | AVYYCLQSQSYPLTFGQGTK |
| | MTIYDGYYVDYALDYWGQG | LEIK |
| | TLVTVSS | |
| 5F7-hu-3 | SEQ ID NO: 171 | SEQ ID NO: 172 |
| | QVQLVESGGGLVKPGGSLRL | DIVMTQSPSSVSASVGDRVTI |
| | SCAASGFAFTDYYMNWIRQA | TCRASQNINIWLSWYQQKPG |
| | PGKGLEWVALIRNKAAGYTI | KAPKLFIYKASNLHTGVPSR |
| | YYSDSVKGRFTISRDNAKSSL | FSGSGSGTDFTLTISSLQPED |
| | YLQMNSLRAEDTAVYYCVRD | FATYYCLQSQSYPLTFGQGT |
| | MTIYDGYYVDYALDYWGQG | KLEIK |
| | TLVTVSS | |
| 5F7-hu-5 | SEQ ID NO: 176 | SEQ ID NO: 177 |
| | EVQLVESGGGLVKPGGSLRLS | DIVMTQSPSSVSASVGDRVTI |
| | CAASGFAFTDYYMNWVRQA | TCRASQNINIWLSWYQQKPG |
| | PGKGLEWVALIRNKAAGYTI | KAPKLFIYKASNLHTGVPSR |
| | YYSAPVKGRFTISRDDSKSTL | FSGSGSGTDFTLTISSLQPED |
| | YLQMNSLKTEDTAVYYCVRD | FATYYCLQSQSYPLTFGQGT |
| | MTIYDGYYVDYALDYWGQG | KLEIK |
| | TLVTVSS | |
| 5F7-2a1 | SEQ ID NO: 179 | SEQ ID NO: 150 |
| | EVQLVESGGGLVKPGGSLRLS | DIVMTQSPSSVSASVGDRVTI |
| | CAASGFDFTDYYMHWVRQA | TCRASRNINIWLSWYQQKPG |
| | PGKGLEWVALIRNKAAGYTI | KAPKLFIYKASNLHTGVPSR |
| | YYSAPVKGRFTISRDDSKSTL | FSGSGSGTDFTLTISSLQPED |
| | YLQMNSLKTEDTAVYYCVRD | FATYYCLQSQSYPLTFGQGT |
| | MTIYDGYYVDYALDYWGQG | KLEIK |
| | TLVTVSS | |
| 5F7-2a6 | SEQ ID NO: 182 | SEQ ID NO: 150 |
| | EVQLVESGGGLVKPGGSLRLS | DIVMTQSPSSVSASVGDRVTI |
| | CAASGFDFTDYYMNWVRQA | TCRASRNINIWLSWYQQKPG |
| | PGKGLEWVALIRNKAAGYTI | KAPKLFIYKASNLHTGVPSR |
| | YYSAPVKGRFTISRDDSKSTL | FSGSGSGTDFTLTISSLQPED |
| | YLQMNSLKTEDTAVYYCVRD | FATYYCLQSQSYPLTFGQGT |
| | MTIYDGYYVDYALDYWGQG | KLEIK |
| | TLVTVSS | |
| 5F7-2a8 | SEQ ID NO: 149 | SEQ ID NO: 150 |
| | EVQLVESGGGLVKPGGSLRLS | DIVMTQSPSSVSASVGDRVTI |
| | CAASGFAFGDYYMNWVRQA | TCRASRNINIWLSWYQQKPG |
| | PGKGLEWVALIRNKAAGYTI | KAPKLFIYKASNLHTGVPSR |
| | YYSAPVKGRFTISRDDSKSTL | FSGSGSGTDFTLTISSLQPED |
| | YLQMNSLKTEDTAVYYCVRD | FATYYCLQSQSYPLTFGQGT |
| | MTIYDGYYVDYALDYWGQG | KLEIK |
| | TLVTVSS | |
| 5F7-2c4 | SEQ ID NO: 183 | SEQ ID NO: 150 |
| | EVQLVESGGGLVKPGGSLRLS | DIVMTQSPSSVSASVGDRVTI |
| | CAASGFAFTDYYLRWVRQAP | TCRASRNINIWLSWYQQKPG |
| | GKGLEWVALIRNKAAGYTIY | KAPKLFIYKASNLHTGVPSR |
| | YSAPVKGRFTISRDDSKSTLY | FSGSGSGTDFTLTISSLQPED |
| | LQMNSLKTEDTAVYYCVRD | FATYYCLQSQSYPLTFGQGT |
| | MTIYDGYYVDYALDYWGQG | KLEIK |
| | TLVTVSS | |
| 5F7-2d3 | SEQ ID NO: 185 | SEQ ID NO: 150 |
| | EVQLVESGGGLVKPGGSLRLS | DIVMTQSPSSVSASVGDRVTI |
| | CAASGFNFTDYYMNWVRQA | TCRASRNINIWLSWYQQKPG |
| | PGKGLEWVALIRNKAAGYTI | KAPKLFIYKASNLHTGVPSR |
| | YYSAPVKGRFTISRDDSKSTL | FSGSGSGTDFTLTISSLQPED |
| | YLQMNSLKTEDTAVYYCVRD | FATYYCLQSQSYPLTFGQGT |
| | MTIYDGYYVDYALDYWGQG | KLEIK |
| | TLVTVSS | |
| 5F7-2g10 | SEQ ID NO: 187 | SEQ ID NO: 150 |
| | EVQLVESGGGLVKPGGSLRLS | DIVMTQSPSSVSASVGDRVTI |
| | CAASGFAFTDYYLSWVRQAP | TCRASRNINIWLSWYQQKPG |
| | GKGLEWVALIRNKAAGYTIY | KAPKLFIYKASNLHTGVPSR |
| | YSAPVKGRFTISRDDSKSTLY | FSGSGSGTDFTLTISSLQPED |
| | LQMNSLKTEDTAVYYCVRD | FATYYCLQSQSYPLTFGQGT |
| | MTIYDGYYVDYALDYWGQG | KLEIK |
| | TLVTVSS | |
| 5F7-2h1 | SEQ ID NO: 189 | SEQ ID NO: 150 |
| | EVQLVESGGGLVKPGGSLRLS | DIVMTQSPSSVSASVGDRVTI |
| | CAASGYAFTDYYMNWVRQA | TCRASRNINIWLSWYQQKPG |
| | PGKGLEWVALIRNKAAGYTI | KAPKLFIYKASNLHTGVPSR |
| | YYSAPVKGRFTISRDDSKSTL | FSGSGSGTDFTLTISSLQPED |
| | YLQMNSLKTEDTAVYYCVRD | FATYYCLQSQSYPLTFGQGT |
| | MTIYDGYYVDYALDYWGQG | KLEIK |
| | TLVTVSS | |

TABLE 5
FR amino acid sequences of 10 humanized antibodies

| Antibody | | FR1 | FR2 | FR3 | FR4 |
|---|---|---|---|---|---|
| 5F7-hu-2 | HFR | SEQ ID NO: | SEQ ID NO: | SEQ ID NO: | SEQ ID NO: |
| | | 165 | 166 | 167 | 157 |
| | | QVQLQESGG | WIRQAPGKG | RFTISRDNA | WGQGTLVT |
| | | GLVKPGGSL | LEWVS | KSSLYLQMN | VSS |
| | | RLSCAASGF | | SLRAEDTAV | |
| | | AFT | | YYCVR | |
| | LFR | SEQ ID NO: | SEQ ID NO: | SEQ ID NO: | SEQ ID NO: |
| | | 168 | 169 | 170 | 161 |
| | | EIVMTQSPA | WYQQKPGQ | GVPARFSGS | FGQGTKLEI |
| | | TLSVSPGER | APRLFIY | GSGTEFTLTI | K |
| | | ATLSC | | SSLQSEDFA | |
| | | | | VYYC | |
| 5F7-hu-3 | HFR | SEQ ID NO: | SEQ ID NO: | SEQ ID NO: | SEQ ID NO: |
| | | 174 | 175 | 167 | 157 |
| | | QVQLVESGG | WIRQAPGKG | RFTISRDNA | WGQGTLVT |
| | | GLVKPGGSL | LEWVA | KSSLYLQMN | VSS |
| | | RLSCAASGF | | SLRAEDTAV | |
| | | AFT | | YYCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-hu-5 | HFR | SEQ ID NO: | SEQ ID NO: | SEQ ID NO: | SEQ ID NO: |
| | | 178 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGF | | LKTEDTAVY | |
| | | AFT | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-2a1 | HFR | SEQ ID NO: | SEQ ID NO: | SEQ ID NO: | SEQ ID NO: |
| | | 181 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGF | | LKTEDTAVY | |
| | | DFT | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-2a6 | HFR | SEQ ID NO: | SEQ ID NO: | SEQ ID NO: | SEQ ID NO: |
| | | 181 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGF | | LKTEDTAVY | |
| | | DFT | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-2a8 | HFR | SEQ ID NO: | SEQ ID NO: | SEQ ID NO: | SEQ ID NO: |
| | | 154 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGF | | LKTEDTAVY | |
| | | AFG | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-2c4 | HFR | SEQ ID NO: | SEQ ID NO: | SEQ ID NO: | SEQ ID NO: |
| | | 178 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGF | | LKTEDTAVY | |
| | | AFT | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-2d3 | HFR | SEQ ID NO: | SEQ ID NO: | SEQ ID NO: | SEQ ID NO: |
| | | 186 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGF | | LKTEDTAVY | |
| | | NFT | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-2g10 | HFR | SEQ ID NO: | SEQ ID NO: | SEQ ID NO: | SEQ ID NO: |
| | | 178 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGF | | LKTEDTAVY | |
| | | AFT | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |
| 5F7-2h1 | HFR | SEQ ID NO: | SEQ ID NO: | SEQ ID NO: | SEQ ID NO: |
| | | 190 | 155 | 156 | 157 |
| | | EVQLVESGG | WVRQAPGK | RFTISRDDSK | WGQGTLVT |
| | | GLVKPGGSL | GLEWVA | STLYLQMNS | VSS |
| | | RLSCAASGY | | LKTEDTAVY | |
| | | AFT | | YCVR | |
| | LFR | SEQ ID NO: | SEQ ID NO: | SEQ ID NO: | SEQ ID NO: |
| | | 158 | 159 | 160 | 161 |
| | | DIVMTQSPS | WYQQKPGK | GVPSRFSGS | FGQGTKLEI |
| | | SVSASVGDR | APKLFIY | GSGTDFTLTI | K |
| | | VTITC | | SSLQPEDFA | |
| | | | | TYYC | |

In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1-4, and/or light chain LFR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the present disclosure also provides humanized anti-IL-36R antibodies and antigen-binding fragments thereof comprising a heavy chain HFR1 comprising the sequence of X₆VQLX₇ESGGGLVKPGGSLRLSCAASGX₈X₉FX₁₀ (SEQ ID NO: 195) or a homologous sequence of at least 80% sequence identity thereof, a heavy chain HFR2 comprising the sequence of WX₁₁RQAPGKGLEWVX₁₂ (SEQ ID NO: 196) or a homologous sequence of at least 80% sequence identity thereof, a heavy chain HFR3 comprising the sequence of RFTISRDX₁₃X₁₄KSX₁₅LYLQMNSLX₁₆X₁₇EDTAVYYCVR (SEQ ID NO: 197) or a homologous sequence of at least 80% sequence identity thereof, and a heavy chain HFR4 comprising the sequence of SEQ ID NO: 157 or a homologous sequence of at least 80% sequence identity thereof, wherein X₆ is Q or E; X₇ is Q or V; X₈ is F or Y; X₉ is A, D or N; X₁₀ is T or G; X₁₁ is I or V; X₁₂ is S or A; X₁₃ is N or D; X₁₄ is A or S; X₁₅ is S or T; X₁₆ is R or K; X₁₇ is A or T.

In certain embodiments, the present disclosure also provides humanized anti-IL-36R antibodies and antigen-binding fragments thereof comprising a light chain LFR1 comprising the sequence of X₂₀IVMTQSPX₂₁X₂₂X₂₃SX₂₄SX₂₅GX₂₆RX₂₇TX₂₈X₂₉C (SEQ ID NO: 198) or a homologous sequence of at least 80% sequence identity thereof, a light chain LFR2 comprising the sequence of WYQQKPGX₃₀APX₃₁LFIY (SEQ ID NO: 199) or a homologous sequence of at least 80% sequence identity thereof, a light chain LFR3 comprising the sequence of GVPX₃₂RFSGSGSGTX₃₃FTLTISSLQX₃₄EDFAX₃₅YYC (SEQ ID NO: 200) or a homologous sequence of at least 80% sequence identity thereof, and a light chain LFR4 comprising the sequence of SEQ ID NO: 161 or a homologous sequence of at least 80% sequence identity thereof, wherein X₂₀ is D or E; X₂₁ is S or A; X₂₂ is S or T; X₂₃ is L or V; X₂₄ is A or V; X₂₅ is V or P; X₂₆ is D or E; X₂₇ is V or A; X₂₈ is I or L; X₂₉ is T or S, X₃₀ is Q or K; X₃₁ is K or R; X₃₂ is S or A; X₃₃ is D or E; X₃₄ is S or P; X₃₅ is T or V.

In certain embodiments, the present disclosure also provides humanized anti-IL-36R antibodies and antigen-binding fragments thereof comprising a heavy chain HFR1 comprising a sequence selected from the group consisting of SEQ ID NOs: 165, 174, 178, 181, 154, 178, 186, 178 and 190, a heavy chain HFR2 comprising a sequence selected from the group consisting of SEQ ID NOs: 166, 175 and 155, a heavy chain HFR3 comprising a sequence selected from the group consisting of SEQ ID NOs: 167 and 156, and a heavy chain HFR4 comprising a sequence of SEQ ID NO: 157; and/or a light chain LFR1 comprising a sequence from the group consisting of SEQ ID NO: 168 and 158, a light chain LFR2 comprising a sequence selected from the group consisting of SEQ ID NOs: 169 and 159, a light chain LFR3 comprising a sequence selected from the group consisting of SEQ ID NOs: 170 and 160, and a light chain LFR4 comprising a sequence of SEQ ID NO: 161.

In certain embodiments, the present disclosure also provides humanized anti-IL-36R antibodies and antigen-binding fragments thereof comprising HFR1, HFR2, HFR3, and/or HFR4 sequences contained in a heavy chain variable region selected from a group consisting of: 5F7-hu-2-VH (SEQ ID NO: 162), 5F7-hu-3-VH (SEQ ID NO: 171), 5F7-hu-5-VH (SEQ ID NO: 176), 5F7-2a1-VH (SEQ ID NO: 179), 5F7-2a6-VH (SEQ ID NO: 182), 5F7-2a8-VH (SEQ ID NO: 149), 5F7-2c4-VH (SEQ ID NO: 183), 5F7-2d3-VH (SEQ ID NO: 185), 5F7-2g10-VH (SEQ ID NO: 187), and 5F7-2h1-VH (SEQ ID NO: 189).

In certain embodiments, the present disclosure also provides humanized anti-IL-36R antibodies and antigen-binding fragments thereof comprising LFR1, LFR2, LFR3, and/or LFR4 sequences contained in a light chain variable region selected from a group consisting of: 5F7-hu-2-VL (SEQ ID NO: 163), 5F7-hu-3-VL (SEQ ID NO: 172), 5F7-hu-5-VL (SEQ ID NO: 177), 5F7-2a1-VL/5F7-2a6-VL/5F7-2a8-VL/5F7-2c4-VL/5F7-2d3-VL/5F7-2g10-VL/5F7-2h1-VL (SEQ ID NO: 150).

In certain embodiments, the humanized anti-IL-36R antibodies and antigen-binding fragments thereof provided herein comprise a heavy chain variable domain sequence selected from the group consisting of SEQ ID NO: 162, SEQ ID NO: 171, SEQ ID NO: 176, SEQ ID NO: 179, SEQ ID NO: 182, SEQ ID NO: 149, SEQ ID NO: 183, SEQ ID NO: 185, SEQ ID NO: 187, and SEQ ID NO: 189; and/or a light chain variable domain sequence selected from the group consisting of SEQ ID NO: 163, SEQ ID NO: 172, SEQ ID NO: 177, and SEQ ID NO: 150.

The present disclosure also provides exemplary humanized antibodies of 5F7, including:
1) “5F7-hu-2” comprising the heavy chain variable region of 5F7-hu-2-VH (SEQ ID NO: 162) and the light chain variable region of 5F7-hu-2-VL (SEQ ID NO: 163);
2) “5F7-hu-3” comprising the heavy chain variable region of 5F7-hu-3-VH (SEQ ID NO: 171), and the light chain variable region of 5F7-hu-3-VL (SEQ ID NO: 172);
3) “5F7-hu-5” comprising the heavy chain variable region of 5F7-hu-5-VH (SEQ ID NO: 176), and the light chain variable region of 5F7-hu-5-VL (SEQ ID NO: 177);
4) “5F7-2a1” comprising the heavy chain variable region of 5F7-2a1-VH (SEQ ID NO: 179), and the light chain variable region of 5F7-2a1-VL (SEQ ID NO: 150);
5) “5F7-2a6” comprising the heavy chain variable region of 5F7-2a6-VH (SEQ ID NO: 182), and the light chain variable region of 5F7-2a6-VL (SEQ ID NO: 150);
6) “5F7-2a8” comprising the heavy chain variable region of 5F7-2a8-VH (SEQ ID NO: 149), and the light chain variable region of 5F7-2a8-VL (SEQ ID NO: 150);
7) “5F7-2c4” comprising the heavy chain variable region of 5F7-2c4-VH (SEQ ID NO: 183), and the light chain variable region of 5F7-2c4-VL (SEQ ID NO: 150);
8) “5F7-2d3” comprising the heavy chain variable region of 5F7-2d3-VH (SEQ ID NO: 185), and the light chain variable region of 5F7-2d3-VL (SEQ ID NO: 150);
9) “5F7-2g10” comprising the heavy chain variable region of 5F7-2g10-VH (SEQ ID NO: 187), and the light chain variable region of 5F7-2g10-VL (SEQ ID NO: 150);
10) “5F7-2h1” comprising the heavy chain variable region of 5F7-2h1-VH (SEQ ID NO: 189), and the light chain variable region of 5F7-2h1-VL (SEQ ID NO: 150).

These exemplary humanized anti-IL-36R antibodies retained the specific binding capacity or affinity to IL-36R, and are at least comparable to, or even better than, the parent mouse antibody 5F7 in that aspect. For example, data is provided in Examples 4-16.

In some embodiments, the anti-IL-36R antibodies and antigen-binding fragments provided herein comprise all or a portion of the heavy chain variable domain and/or all or a portion of the light chain variable domain. In one embodiment, the anti-IL-36R antibody or an antigen-binding fragment thereof provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-IL-36R antibodies or the antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-IL-36R antibodies or the antigen-binding fragments thereof provided herein comprises heavy chain constant regions of human IgG1, IgG2, IgG3, or IgG4. In certain embodiments, the light chain constant region comprises Cκ or Cλ. The constant region of the anti-IL-36R antibodies or the antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-IL-36R antibodies and antigen-binding fragments thereof provided herein comprises an Fc region of IgG1 or IgG3 isotype, which could induce ADCC or CDC; or alternatively, a constant region of IgG4 or IgG2 isotype, which has reduced or depleted effector function. In certain embodiments, the anti-IL-36R antibodies or antigen-binding fragments thereof provided herein comprise a wild type human IgG4 Fc region or other wild type human IgG4 alleles. In certain embodiments, the anti-IL-36R antibodies or antigen-binding fragments thereof provided herein comprise a human IgG4 Fc region comprising an S228P mutation. In certain embodiments, the anti-IL-36R antibodies or antigen-binding fragments thereof provided herein comprise the heavy chain constant region with an amino acid sequence as shown in SEQ ID NO: 203. In certain embodiments, the anti-IL-36R antibodies or antigen-binding fragments thereof provided herein comprise a human IgG4 Fc region comprising an M252Y/S254T/T256E (YTE) mutation. In certain embodiments, the anti-IL-36R antibodies or antigen-binding fragments thereof provided herein comprise a human IgG4 Fc region comprising a T307Q/N434A (QA) mutation. In certain embodiments, the anti-IL-36R antibodies or antigen-binding fragments thereof provided herein comprise a human IgG4 Fc region comprising an S228P mutation, and an M252Y/S254T/T256E (YTE) mutation. In certain embodiments, the anti-IL-36R antibodies or antigen-binding fragments thereof provided herein comprise the heavy chain constant region with an amino acid sequence as shown in SEQ ID NO: 201. In certain embodiments, the anti-IL-36R antibodies or antigen-binding fragments thereof provided herein comprise a human IgG4 Fc region comprising an S228P mutation, and a T307Q/N434A (QA) mutation. In certain embodiments, the anti-IL-36R antibodies or antigen-binding fragments thereof provided herein comprise the heavy chain constant region with an amino acid sequence as shown in SEQ ID NO: 204. In certain embodiments, the anti-IL-36R antibodies or antigen-binding fragments thereof provided herein comprise a human IgG4 Fc region comprising a S228P mutation, an M252Y/S254T/T256E (YTE) mutation and a T307Q/N434A (QA) mutation.

In certain embodiments, the antibodies or the antigen-binding fragments thereof provided herein have a specific binding affinity to human IL-36R which is sufficient to provide for diagnostic and/or therapeutic use.

The antibodies or antigen-binding fragments thereof provided herein can be a monoclonal antibody, a polyclonal antibody, a humanized antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-IL-36R antibody or antigen-binding fragment thereof, which does not competes for binding to human IL-36R with an antibody selected from the group consisting of: a) an antibody comprising a heavy chain variable region comprising the sequence of SEQ ID NO: 205, and a light chain variable region comprising the sequence of SEQ ID NO: 206; b) an antibody comprising a heavy chain variable region comprising the sequence of SEQ ID NO: 209, and a light chain variable region comprising the sequence of SEQ ID NO: 210, and wherein the antibody or an antigen-binding fragment thereof of is not BI655130 or REGN14.

“BI655130” as used herein refers to an antibody or antigen binding fragment thereof comprising a heavy chain variable region having an amino acid sequence of SEQ ID NO: 205, and a light chain variable region having an amino acid sequence of SEQ ID NO: 206.

“REGN14” as used herein refers to an antibody or antigen binding fragment thereof comprising a heavy chain variable region having an amino acid sequence of SEQ ID NO: 209, and a light chain variable region having an amino acid sequence of SEQ ID NO: 210.

### Antibody Variants

The antibodies and antigen-binding fragments thereof provided herein also encompass various variants of the antibody sequences provided herein.

In certain embodiments, the antibody variants comprise one or more modifications or substitutions in one or more of the CDR sequences as provided in Tables 1 and 3 above, one or more of the non-CDR sequences of the heavy chain variable region or light chain variable region provided in Tables 2 and 4 above, and/or the constant region (e.g., Fc region). Such variants retain binding specificity to IL-36R of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g., one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variants

Affinity variants of antibodies may contain modifications or substitutions in one or more CDR sequences as provided in Tables 1 and 3 above, one or more FR sequences as provided in Table 5 above, or the heavy or light chain variable region sequences provided in Tables 2 and 4 above. FR sequences can be readily identified by a person skilled in the art based on the CDR sequences in Tables 1 and 3 above and variable region sequences in Tables 2 and 4 above, as it is well-known in the art that a CDR region is flanked by two FR regions in the variable region. The affinity variants retain specific binding affinity to IL-36R of the parent antibody, or even have improved IL-36R specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Tables 1 and 3 above, and variable region sequences provided in Tables 2 and 4 above, one or more amino acid residues may be substituted yet the resulting antibody or antigen-binding fragment still retain the binding affinity or binding capacity to IL-36R, or even have an improved binding affinity or capacity. Various methods known in the art can be used to achieve this purpose. For example, a library of antibody variants (such as Fab or scFv variants) can be generated and expressed with phage display technology, and then screened for the binding affinity to human IL-36R. For another example, computer software can be used to virtually simulate the binding of the antibodies to human IL-36R, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitution(s) in the CDR sequences and/or FR sequences in total.

In certain embodiments, the anti-IL-36R antibodies or antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Tables 1 and 3 above yet retaining the specific binding affinity to IL-36R at a level similar to or even higher than its parent antibody.

In certain embodiments, the anti-IL-36R antibodies or antigen-binding fragments thereof comprise one or more variable region sequences having at least 80% (e.g., at least 85%, 88%, 90%, 91%, 92%, 93%, 94%, 95%, 96%, 97%, 98%, 99%) sequence identity to that (or those) listed in Tables 2 and 4 above yet retaining the specific binding affinity to IL-36R at a level similar to or even higher than its parent antibody. In some embodiments, a total of 1 to 10 amino acids have been substituted, inserted, or deleted in a variable region sequence listed in Tables 2 and 4 above. In some embodiments, the substitutions, insertions, or deletions occur in regions outside the CDRs (e.g., in the FRs).

### Glycosylation Variants

The anti-IL-36R antibodies or antigen-binding fragments thereof provided herein also encompass glycosylation variants, which can be obtained to either increase or decrease the extent of glycosylation of the antibodies or antigen binding fragments thereof.

The antibodies or antigen binding fragments thereof may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

In certain embodiments, the anti-IL-36R antibodies and antigen-binding fragments provided herein comprise a mutation at N297 (e.g., N297A, N297Q, or N297G) to remove the glycosylation site.

### Cysteine-Engineered Variants

The anti-IL-36R antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-IL-36R antibodies or antigen-binding fragments thereof provided herein also encompass Fc variants, which comprise one or more amino acid residue modifications or substitutions at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O., et al., Mol Cancer Ther. 2008, 7(8): 2517-27; Shields R. L. et al., J. Biol. Chem, 2002, 277: 26733-26740; Shinkawa T. et al., J. Biol. Chem, 2003, 278: 3466-3473.

CDC activity of the antibodies or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821; and WO94/29351 concerning other examples of Fc region variants). One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-IL-36R antibodies or antigen-binding fragments thereof provided herein has reduced effector functions, and comprise one or more amino acid substitution(s) in IgG1 at a position selected from the group consisting of: 234, 235, 237, and 238, 268, 297, 309, 330, and 331. In certain embodiments, the anti-IL-36R antibodies or antigen-binding fragments thereof provided herein is of IgG1 isotype and comprise one or more amino acid substitution(s) selected from the group consisting of: N297A, N297Q, N297G, L235E, L234A, L235A, L234F, L235E, P331S, and any combination thereof. In certain embodiments, the anti-IL-36R antibodies or antigen-binding fragments thereof provided herein is of IgG2 isotype, and comprises one or more amino acid substitution(s) selected from the group consisting of: H268Q, V309L, A330S, P331S, V234A, G237A, P238S, H268A, and any combination thereof (e.g., H268Q/V309L/A330S/P331S, V234A/G237A/P238S/H268A/V309L/A330S/P331S). In certain embodiments, the anti-IL-36R antibodies or antigen-binding fragments thereof provided herein is of IgG4 isotype, and comprises one or more amino acid substitution(s) selected from the group consisting of: N297A, N297Q, N297G, L235E, L234A, L235A, M252Y/S254T/T256E, T307Q/N434 and any combination thereof. In certain embodiments, the anti-IL-36R antibodies or antigen-binding fragments thereof provided herein is of IgG2/IgG4 cross isotype. Examples of IgG2/IgG4 cross isotype is described in Rother R P et al., Nat Biotechnol 25:1256-1264 (2007).

In certain embodiments, the anti-IL-36R antibodies and antigen-binding fragments provided herein is of IgG4 isotype and comprises the amino acid substitutions of M252Y/S254T/T256 (YTE). In certain embodiments, the anti-IL-36R antibodies and antigen-binding fragments provided herein is of IgG4 isotype and comprises the amino acid substitutions of T307Q/N434A (QA).

In certain embodiments, the anti-IL-36R antibodies and antigen-binding fragments provided herein is of IgG4 isotype and comprises one or more amino acid substitution(s), for example at the point of 228. In certain embodiments, the anti-IL-36R antibodies and antigen-binding fragments provided herein is of IgG4 isotype and comprises S228P mutation in the Fc region. In certain embodiments, the anti-IL-36R antibodies or antigen-binding fragments thereof provided herein comprise the heavy chain constant region with an amino acid sequence as shown in SEQ ID NO: 203. In certain embodiments, the anti-IL-36R antibodies and antigen-binding fragments provided herein is of IgG4 isotype and comprises the amino acid substitutions of S228P mutation and M252Y/S254T/T256 (YTE) mutation. In certain embodiments, the anti-IL-36R antibodies or antigen-binding fragments thereof provided herein comprise the heavy chain constant region with an amino acid sequence as shown in SEQ ID NO: 201. In certain embodiments, the anti-IL-36R antibodies and antigen-binding fragments provided herein is of IgG4 isotype and comprises the amino acid substitutions of S228P mutation and T307Q/N434A (QA) mutation. In certain embodiments, the anti-IL-36R antibodies or antigen-binding fragments thereof provided herein comprise the heavy chain constant region with an amino acid sequence as shown in SEQ ID NO: 204.

In certain embodiments, the anti-IL-36R antibodies or antigen-binding fragments thereof comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved P K, published by Springer, 2010; Yeung, Y. et al., Cancer Research, 70: 3269-3277 (2010); and Hinton, P. et al., J. Immunology, 176:346-356 (2006).

In certain embodiments, anti-IL-36R antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g. as described in U.S. Pat. No. 5,731,168.

### Antigen-Binding Fragments

Provided herein are also anti-IL-36R antigen-binding fragments. Various types of antigen-binding fragments are known in the art and can be developed based on the anti-IL-36R antibodies provided herein, including for example, the exemplary antibodies whose CDRs are shown in Tables 1 and 3 above, and variable sequences are shown in Tables 2 and 4 above, and their different variants (such as affinity variants, glycosylation variants, Fc variants, cysteine-engineered variants and so on).

In certain embodiments, an anti-IL-36R antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-IL-36R antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the anti-IL-36R antibodies or antigen-binding fragments thereof is bispecific. In certain embodiments, the antibody or antigen-binding fragment thereof is further linked to a second functional moiety having a different binding specificity from said IL-36R antibody, or antigen binding fragment thereof.

In certain embodiments, the bispecific antibodies or antigen-binding fragments thereof provided herein are capable of specifically binding to a second antigen other than IL-36R, or a second epitope on IL-36R. In some embodiments, the second antigen other than IL-36R is selected from the group consisting of IL-17, IL-23, TNF, IL-12, and IL-1.

### Conjugates

In some embodiments, the anti-IL-36R antibodies or antigen-binding fragments thereof further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding, affinity binding, intercalation, coordinate binding, complexation, association, blending, or addition, among other methods. In some embodiments, the antibodies or antigen-binding fragments thereof can be linked to one or more conjugates via a linker.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In some embodiments, the antibody moiety is linked with the conjugate moiety through a chemical bond or a linker. In some embodiments, the antibody moiety and the conjugate moiety is linked using a variety of well-known bifunctional reagents and chemistries suitable for conjugating to proteins. Such reagents include but are not limited to: N-succinimidyl-3-(2-pyridyldithio) propionate (SPDP), succinimidyl-4-(N-maleimidornethyl) cyclohexane-1-carboxylate (SMCC), iminothiolane (IT), bifunctional derivatives of imidoesters (e.g., dimethyl adipimidate HQ), active esters (e.g., disuccinimidyl suberate), aldehydes (e.g., glutaraldehyde), bis-azido compounds bis-(p-azidobenzoyl)-hexane-diamine), bis-diazonium derivatives (e.g., bis-(p-diazoniumbenzoyl)-ethylenediamine), diisocyanates (e.g., toluene-2,6-diisocyanate), and bis-active fluorine compounds (e.g., 1,5-difluoro-2,4-dinitrobenzene).

In certain embodiments, the antibodies or antigen-binding fragments thereof may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g. a polymer such as PEG which extends half-life), a chemotherapeutic agent, a toxin, a radioactive isotope, a lanthanide, a detectable label (e.g. a luminescent label, a fluorescent label, an enzyme-substrate label), a DNA-alkylator, a topoisomerase inhibitor, a tubulin-binder, a purification moiety or other therapeutic agents or drugs.

The therapeutic agents or drugs useful as the conjugate moiety can be those which are useful for treating psoriasis, e.g. GPP (generalized pustular psoriasis), PPP (palmoplantar pustulosis), HS (hidradenitis suppurativa) and the like.

In some embodiments, the conjugate moiety comprises a hormone-based immunosuppressant. In some embodiments, the conjugate moiety comprises glucocorticoids or steroids. Non-limiting exemplary glucocorticoids or steroids include budesonide, flunisolide, triamcinolone acetonide, fluticasone propionate, beclomethasone diproprionate, and ciclesonide.

In some embodiments, the conjugate moiety comprises a therapeutic agent or drug for treating psoriasis. In some embodiments, the conjugate moiety can be a non-biologic agent such as methotrexate, ciclosporin, fumaric acid esters (FAE), hydroxycarbamide, fumarates (such as dimethyl fumarate), retinoids (synthetic forms of vitamin A), glucocorticoids or steroids. In some embodiments, the conjugate moiety can be a biologic agent that interrupts the immune process involved in psoriasis, targeting specific aspects of the immune system contributing to psoriasis. In some embodiments, the biologic agent is a monoclonal antibody or an antigen-binding fragment thereof, targeting anti-IL17, anti-IL12/23, anti-IL23, and/or anti-TNF alpha, etc. Examples of such monoclonal antibody include but are not limited to infliximab, adalimumab, golimumab, certolizumab pegol, ixekizumab, ustekinumab, guselkumab, efalizumab, alefacept, secukinumab, and brodalumab.

In some embodiments, the conjugate moiety comprises a therapeutic agent or drug for treating GPP (generalized pustular psoriasis). In some embodiments, the conjugate moiety comprises a therapeutic agent or a drug such as a glucocorticoid or steroid, etanercept, PUVA, hydroxyurea, dapsone, cyclosporin A, adalimumab, etretinate, isotretinoin, or acitretin.

In some embodiments, the conjugate moiety comprises a therapeutic agent or drug for treating PPP (palmoplantar pustulosis). In some embodiments, the conjugate moiety comprises a therapeutic agent or a drug such as a retinoid, ciclosporin, a tetracycline, colchicine, Tripterygium wilfordii, Tripterygium hypoglaucum hutch, or an anti-interleukin 23 monoclonal antibody (such as guselkumab) or an antigen-binding fragment thereof.

In some embodiments, the conjugate moiety comprises a therapeutic agent or drug for treating HS (hidradenitis suppurativa). In some embodiments, the conjugate moiety comprises a therapeutic agent or a drug such as a corticosteroid, an antibiotic, an anti-androgenic agent, or an anti-inflammatory agent. Examples of antibiotics include but are not limited to rifampicin, clindamycin, tetracycline and minocycline. Examples of anti-androgenic agent include but are not limited to spironolactone, flutamide, cyproterone acetate, ethinylestradiol, finasteride, dutasteride, and metformin. Examples of the anti-inflammatory agent include but are not limited to a TNF inhibitor, such as infliximab, etanercept and adalimumab.

In some embodiments, the conjugate moiety comprises an enzymatically active toxin or a fragment thereof, including but not limited to diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), anti-mitotic agents (e.g. vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioactive isotopes, luminescent labels, chromophoric moieties, digoxigenin, biotin/avidin, DNA molecules or gold for detection. A variety of radioactive isotopes are available for the production of such radio-conjugates. Examples include ²¹¹At, ¹³¹I, ¹²⁵I ⁹⁰Y, ¹⁸⁶Re, ¹⁸⁸Re, ¹⁵³Sm, ²¹²Bi, ³²P, ²¹²Pb, and radioactive isotopes of Lu. In some embodiments, the conjugate moiety may comprise a radioisotope for scintigraphic detection, or a spin label for NMR detection or MRI. Suitable radioisotopes or spin labels can include, as ¹²³I, ¹³¹I, ¹¹¹In, ¹³C, ¹⁹F, ¹⁵N, ¹⁷O, various isotopes of Gd, Mn, and Fe.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer is attached, they can be the same or different molecules.

In certain embodiments, the conjugate moiety can be a purification moiety such as a magnetic bead.

In certain embodiments, the antibodies or antigen-binding fragments thereof provided herein is used as a base for a conjugate.

### Polynucleotides and Recombinant Methods

The present disclosure provides isolated polynucleotides that encode the anti-IL-36R antibodies or antigen-binding fragments thereof provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The isolated polynucleotide that encodes the anti-IL-36R antibodies or antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

The present disclosure provides vectors comprising the isolated polynucleotide provided herein. In certain embodiments, the polynucleotide provided herein encodes the antibodies or antigen-binding fragments thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-TL-36R antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; Yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for anti-IL-36R antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-IL-36R antibodies or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the anti-IL-36R antibodies or antigen-binding fragments thereof and one or more pharmaceutically acceptable carriers.

The present disclosure further provides a pharmaceutical composition comprising the polynucleotides encoding the anti-IL-36R antibodies or antigen-binding fragments thereof, and one or more pharmaceutically acceptable carriers. Antibodies provided herein can also be produced in vivo by delivery of polynucleotides encoding the antibodies or antigen-binding fragments thereof provided herein, such as, for example, in-vitro-transcribed mRNA, or expression vectors. Methods are known in the art for polynucleotide delivery for antibody expression in vivo, see, for example, Rybakova, Y. et al, Molecular Therapy, vol. 27 (8), pp. 1415-1423 (2019); Deal, C. E. et al, Vaccines, 2021, 9, 108.

The present disclosure further provides pharmaceutical compositions comprising an expression vector comprising the polynucleotides encoding the anti-IL-36R antibodies or antigen-binding fragments thereof, and one or more pharmaceutically acceptable carriers.

In certain embodiments, the expression vector comprises a viral vector or a non-viral vector. Examples of viral vectors include, without limitation, adeno-associated virus (AAV) vector, lentivirus vector, retrovirus vector, and adenovirus vector. Examples of non-viral vectors include, without limitation, naked DNA, plasmid, exosome, mRNA, and so on. In certain embodiments, the expression vector is suitable for gene therapy in human. Suitable vectors for gene therapy include, for example, adeno-associated virus (AAV), or adenovirus vector. In certain embodiments, the expression vector comprises a DNA vector or a RNA vector. In certain embodiments, the pharmaceutically acceptable carriers are polymeric excipients, such as without limitation, microspheres, microcapsules, polymeric micelles and dendrimers. The polynucleotides, or polynucleotide vectors of the present disclosure may be encapsulated, adhered to, or coated on the polymer-based components by methods known in the art (see for example, W. Heiser, Nonviral gene transfer techniques, published by Humana Press, 2004; U.S. Pat. No. 6,025,337; Advanced Drug Delivery Reviews, 57(15): 2177-2202 (2005)).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-IL-36R antibody or antigen-binding fragment thereof or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the antibody or an antigen-binding fragment thereof provided herein. In certain embodiments, the present disclosure provides a kit comprising the antibody or an antigen-binding fragment thereof provided herein, and a second therapeutic agent.

In some embodiments, the second therapeutic agent can be those which are useful for treating psoriasis, e.g. GPP (generalized pustular psoriasis), PPP (palmoplantar pustulosis), HS (hidradenitis suppurativa), Behcet's disease, and the like.

In some embodiments, the second therapeutic agent is a hormone-based immunosuppressant. In some embodiments, the second therapeutic agent comprises glucocorticoids or steroids. Non-limiting exemplary glucocorticoids or steroids include budesonide, flunisolide, triamcinolone acetonide, fluticasone propionate, beclomethasone diproprionate, and ciclesonide.

In some embodiments, the second therapeutic agent is a therapeutic agent or drug for psoriasis. In some embodiments, the second therapeutic agent can be a non-biologic agent, such as methotrexate, ciclosporin, fumaric acid esters (FAE), hydroxycarbamide, fumarates (such as dimethyl fumarate), retinoids (synthetic forms of vitamin A), glucocorticoids or steroids. In some embodiments, the second therapeutic agent can be a biologic agent, that interrupt the immune process involved in psoriasis, targeting specific aspects of the immune system contributing to psoriasis. In some embodiments, the biologic agent is a monoclonal antibody targeting anti-IL17, anti-IL12/23, anti-IL23, and/or anti-TNF alpha, etc. Examples of such monoclonal antibody include but are not limited to infliximab, adalimumab, golimumab, certolizumab pegol, ixekizumab, ustekinumab, guselkumab, efalizumab, alefacept, secukinumab, and brodalumab.

In some embodiments, the second therapeutic agent is a therapeutic agent or drug for GPP (generalized pustular psoriasis). In some embodiments, the second therapeutic agent comprises a therapeutic agent or a drug such as glucocorticoid or steroid, etanercept, PUVA, hydroxyurea, dapsone, cyclosporin A, adalimumab, etretinate, isotretinoin, acitretin.

In some embodiments, the second therapeutic agent is a therapeutic agent or drug for PPP (palmoplantar pustulosis). In some embodiments, the second therapeutic agent comprises a therapeutic agent or a drug such as a retinoid, ciclosporin, tetracycline, colchicine, Tripterygium wilfordii, Tripterygium hypoglaucum hutch, an anti-interleukin 23 monoclonal antibody (such as guselkumab).

In some embodiments, the second therapeutic agent is a therapeutic agent or drug for HS (hidradenitis suppurativa). In some embodiments, the second therapeutic agent comprises a therapeutic agent or a drug such as corticosteroid, an antibiotic, an anti-androgenic agent, an anti-inflammatory agent. Examples of antibiotics include but are not limited to rifampicin, clindamycin, tetracycline and minocycline. Examples of anti-androgenic agent include but are not limited to spironolactone, flutamide, cyproterone acetate, ethinylestradiol, finasteride, dutasteride, and metformin. Examples of anti-inflammatory agent include but are not limited to a TNF inhibitor, such as infliximab, etanercept and adalimumab.

In some embodiments, the second therapeutic agent is a therapeutic agent or drug for Behcet's disease. In some embodiments, the second therapeutic agent comprises a therapeutic agent or a drug such as topical drugs, corticosteroids, methotrexate, colchicine, thalidomide, azathioprine, cyclophosphamide, cyclosporine, mycophenolate mofetil and anti-tumor necrosis factor antagonists. Examples of anti-tumor necrosis factor antagonist include but are not limited to infliximab, etanercept and apremilast.

In some embodiments, the second therapeutic agent is a drug targeting IL-17, IL-23, TNF, IL-12, IL-1, etc.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Chimeric Antigen Receptor (CAR) Composition

The present disclosure also provides chimeric antigen receptors (CARs) comprising an anti-IL-36R antigen binding domain as provided herein and a T-cell activation domain. Chimeric antigen receptors (CARs) are engineered chimeric receptors that combine an antigen-binding domain of an antibody with one or more signaling domains for T cell activation. Immune cells such as T cells and Nature Killer (NK) cells can be genetically engineered to express CARs. T cells expressing a CAR are referred to as CAR-T cells. CAR can mediate antigen-specific cellular immune activity in the T cells, enabling the CAR-T cells to eliminate cells (e.g. tumor cells) expressing the targeted antigen. In one embodiment, binding of the CAR-T cells provided herein to IL-36R expressed on cells such as cancer cells, results in proliferation and/or activation of said CAR-T cells, wherein said activated CAT-T cells can release cytotoxic factors, e.g. perforin, granzymes, and granulysin, and initiate cytolysis and/or apoptosis of the cancer cells.

In some embodiments, the T-cell activation domain of the CAR comprises a co-stimulatory signaling domain and a TCR signaling domain, which can be linked to each other in a random or in a specified order, optionally with a short peptide linker having a length of, for example, between 2 and 10 amino acids (e.g. glycine-serine doublet linker).

In some embodiment, the CAR further comprises a transmembrane domain. When expressed in cells, the anti-IL-36R antigen binding domain is extracellular, and the T-cell activation domain is intracellular.

In certain embodiments, the CAR comprises an anti-IL-36R antigen binding domain, a transmembrane domain, a costimulatory signaling region, and a TCR signaling domain, wherein the antigen binding domain specifically binds to IL-36R and comprises an antigen-binding fragment of the antibodies provided herein.

### 1. Antigen Binding Domain

In some embodiments, the anti-IL-36R antigen binding domain of the CAR comprises one or more CDR sequences as provided herein, one or more heavy chain variable domains or light chain variable domains provided herein, or one or more antigen-binding fragment derived from any of the anti-IL-36R antibodies provided herein.

In some embodiments, it is beneficial for the antigen binding domain to be derived from the same species in which the CAR will ultimately be used in. For example, for use in humans, it may be beneficial to have the antigen binding domain used in the CAR derived from a human antibody or a humanized antibody. In some embodiments, the antigen binding domain comprises a single chain variable fragment (scFv). In some embodiment, the antigen binding domain may exist in a variety of other forms including, for example, Fv, Fab, and (Fab′)₂, as well as bi-functional (i.e. bi-specific) hybrid antibody fragments (e.g., Lanzavecchia et al., Eur. J. Immunol. 17, 105 (1987)). In certain embodiments, the antigen binding domain comprises a Fab or a scFv.

### 2. Transmembrane Domain

In certain embodiments, the CAR comprises a transmembrane domain fused to the extracellular antigen-binding domain of the CAR. In one embodiment, the transmembrane domain can be selected such that it is naturally associated with one of the domains in the CAR. In some instances, the transmembrane domain can be selected or modified to avoid binding to transmembrane domains of other members of the T cell receptor complex.

The transmembrane domain of the CAR provided herein may be derived from transmembrane domains of any natural membrane-bound or transmembrane protein, such as, for example, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, and CD154. In some embodiments, the transmembrane domain of the CAR can also use a variety of human hinges such as human Ig (immunoglobulin) hinge.

Alternatively, the transmembrane domain of the CAR provided herein may be synthetic, for example, comprising predominantly hydrophobic residues such as leucine and valine. In one embodiment, a triplet of phenylalanine, tryptophan and valine is included at each end of a synthetic transmembrane domain. Optionally, a short oligo- or polypeptide linker, between 2 and 10 amino acids in length may form the linkage between the transmembrane domain and the intracellular signaling domain of the CAR. A glycine-serine doublet provides a particularly suitable linker.

### 3. TCR Signaling Domain

The T-cell activation domain of the CARs provided herein comprises a TCR signaling domain. The TCR signaling domain can activate the T cell which expresses the CAR, to exert at least one of the normal TCR effector functions of a T cell, for example, cytolytic activity or helper activity including the secretion of cytokines. The TCR signaling domain can be either full-length of a natural intracellular signal transduction domain, or a fragment thereof sufficient to transduce the TCR effector function signal.

Exemplary intracellular signaling domains useful in the CARs provided herein include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

The TCR signaling domain that acts in a stimulatory manner may contain signaling motifs which are known as immunoreceptor tyrosine-based activation motifs or ITAMs. Examples of ITAM containing TCR signaling domains useful in the CAR provided herein include those derived from TCR zeta, FcR gamma, FcR beta, CD3 gamma, CD3 delta, CD3 epsilon, CD5, CD22, CD79a, CD79b, and CD66d. In certain embodiments, the TCR signaling domain comprises a cytoplasmic signaling sequence derived from CD3-zeta.

### 4. Co-Stimulatory Signaling Region

In certain embodiments, the T-cell activation domain of the CARs provided herein may further comprise a co-stimulatory signaling region. Co-stimulatory signaling region acts in an antigen-independent manner to mediate TCR activation, and can be derived from a co-stimulatory molecule required for an efficient response of lymphocytes to an antigen. Exemplary co-stimulatory molecules include, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

### 5. Polynucleotide Sequence Encoding the CAR

In one aspect, the present disclosure further provides nucleic acid sequences encoding the CAR provided herein, comprising a first polynucleotide sequence encoding the antigen binding domain of the CAR provided herein, and optionally a second polynucleotide sequence encoding the transmembrane domain and the T-cell activation domain provided herein. In some embodiments, the sequence encoding the antigen binding domain is operably linked to the sequence encoding the transmembrane domain and the T-cell activation domain. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

In one aspect, the present disclosure provides vectors comprising the nucleic acid sequence encoding the CAR provided herein. In some embodiments, the vector is retroviral and lentiviral vector construct expressing the CAR of the present disclosure which can be directly transduced into a cell, or RNA construct that can be directly transfected into a cell.

In one aspect, the present disclosure provides isolated cells which comprises the nucleic acid sequence encoding the CAR and/or express the CAR provided herein.

In certain embodiments, the cell comprising the nucleic acid encoding the CAR or expressing the CAR is selected from the group consisting of a T cell, a NK cell, a cytotoxic T lymphocyte (CTL), and a regulatory T cell. In one embodiment, the cell comprising the nucleic acid encoding the CAR or expressing the CAR exhibits an antitumor immunity when the antigen binding domain of the CAR binds to its corresponding antigen. The cytotoxic lymphocytes will preferably be autologous cells, although heterologous cells or allogenic cells can be used. As used herein, “autologous” means any material derived from the same individual to whom it is later to be re-introduced into the individual.

In one aspect, the present disclosure further provides methods for stimulating a T cell-mediated immune response to an IL-36R-expressing cell or tissue in a subject, the method comprising administering to the subject an effective amount of a cell genetically modified to express the CAR provided herein.

In one aspect, the present disclosure further provides methods for treating a mammal having a disease, disorder or condition associated with an elevated expression of IL-36R, comprising administering to the mammal an effective amount of a cell genetically modified to express the CAR provided herein, thereby treating the mammal. In certain embodiments, the cell is an autologous T cell. In certain embodiments, the mammal has been diagnosed with the disease, disorder or condition associated with an elevated expression of IL-36R.

### Methods of Use

In another aspect, methods are provided to treat a disease, disorder or condition in a subject that would benefit from modulation of IL-36R activity. In another aspect, methods are provided to treat an IL-36R related disease or disorder in a subject in need thereof. In another aspect, methods are provided to treat a disease, disorder or condition that is responsive to IL-36R inhibition in a subject in need thereof.

In some embodiments, the method comprises administering to the subject a therapeutically effective amount of the antibody or antigen-binding fragment thereof provided herein, or the polynucleotide encoding the antibody or antigen-binding fragment thereof provided herein and/or the pharmaceutical composition provided herein. In certain embodiments, the subject is human.

It is known that IL-36R play a role in the pathogenesis of several diseases such as psoriasis etc. For example, experimental results have shown that IL-36 (α, β, and γ) are elevated in serum and lesional skin in psoriasis patients and correlated with disease activity, and IL-36Ra deficiency or over-expression of IL-36 agonist ligands can lead to pustular psoriasis. IL-36R signaling is a key driver in pustular psoriasis. IL-36R signaling plays central role in hidradenitis suppurativa. Increased levels of IL-36 (α, β, and γ) mRNA and widespread expression of IL-36R in lesional skin of ichthyosis patients.

In some embodiments, the IL-36R related disease or disorder is an IL-36R-positive disease or disorder. In some embodiments, the subject to be treated has been identified as having an IL-36R-positive disease or disorder. In some embodiments, the IL-36R related disease, disorder or condition is responsive to IL-36R inhibition. In some embodiments, the IL-36R related disease, disorder or condition is associated with dysregulation of IL-36R mediated signaling, or more specifically, associated with up-regulated IL-36R signaling.

In some embodiments, the disease or disorder is associated with cells dysregulation of IL-36R mediated signaling. In some embodiments, the dysregulation of IL-36R mediated signaling includes dysregulation of IL-36 (e.g. IL-36α, IL-36β, or IL-36γ), IL-36R antagonist and/or IL-38. In some embodiments, the dysregulation of IL-36R mediated signaling includes over activation of IL-36 (e.g. IL-36α, IL-36β, or IL-36γ), or over suppression of IL-36R antagonist or over suppression of IL-38, compared with control level (e.g., the level in a healthy subject).

In some embodiments, the disease or disorder is an inflammatory disease, an autoimmune disease, a respiratory disease, a metabolic disorder, or cancer.

In some embodiments, the inflammatory disease is selected from the group consisting of: allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, and other allergies. In some embodiments, the inflammatory disease is selected from the group consisting of allergic inflammation of the skin, lungs, and gastrointestinal tract, atopic dermatitis (also known as atopic eczema), asthma (allergic and non-allergic), and epithelial-mediated inflammation.

In some embodiments, the autoimmune disease is selected from the group consisting of: multiple sclerosis, asthma, type 1 diabetes mellitus, rheumatoid arthritis, scleroderma, Crohn's disease, psoriasis vulgaris (commonly referred to as psoriasis), hidradenitis suppurativa, generalized pustular psoriasis (GPP), palmo-plantar pustulosis (PPP), inflammatory bowel disease, psoriatic arthritis, systemic lupus erythematosus (SLE), ulcerative colitis, ankylosing spondylitis atopic dermatitis and acne vulgaris. In some embodiments, the method provided herein is useful to treat pustular psoriasis, generalized pustular psoriasis, palmo-plantar pustulosis (PPP), psoriasis vulgaris, atopic dermatitis and acne vulgaris. In some embodiments, the autoimmune disease is selected from the group consisting of psoriasis vulgaris (commonly referred to as psoriasis), hidradenitis suppurativa, generalized pustular psoriasis (GPP), palmo-plantar pustulosis (PPP), inflammatory bowel disease, atopic dermatitis, acne vulgaris, and Behcet's disease.

In some embodiments, the respiratory disease is selected from the group consisting of asthma, cystic fibrosis, emphysema, chronic obstructive pulmonary disease (COPD), and acute respiratory distress syndrome.

In some embodiments, the metabolic disease is selected from the group consisting of obesity, type 2 diabetes, atherosclerosis, and cardiovascular disease.

In some embodiments, the cancer can be any type of cancer known in the art, including but not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, leukemia, lymphoma, and Merkel cell carcinoma.

In some embodiments, the disease or disorder is selected from the group consisting of psoriasis, pustular psoriasis, hidradenitis suppurativa, ichthyosis, inflammatory bowel disease (IBD), atopic dermatitis, acne vulgaris, and Behcet's disease.

The presence and/or amount of IL-36R in an interested biological sample can be indicative of whether the subject from whom the biological sample is derived could likely respond to an anti-IL-36R antibody. Various methods can be used to determine the presence and/or amount of IL-36R in a test biological sample from the subject. For example, the test biological sample can be exposed to anti-IL-36R antibody or antigen-binding fragment thereof, which binds to and detects the expressed IL-36R protein. Alternatively, IL-36R can also be detected at nucleic acid expression level, using methods such as qPCR, reverse transcriptase PCR, microarray, serial analysis of gene expression (SAGE), fluorescence in situ hybridization (FISH), and the like. In some embodiments, the test sample is derived from an epithelial tissue. In certain embodiments, presence or up-regulated level of the IL-36R in the test biological sample indicates likelihood of responsiveness. The term “up-regulated” as used herein, refers to an overall increase of no less than 10%, 15%, 20%, 25%, 30%, 35%, 40%, 45%, 50%, 55%, 60%, 65%, 70%, 75%, 80% or greater, in the expression level of IL-36R in the test sample, as compared to the IL-36R expression level in a reference sample as detected using the same method. The reference sample can be a control sample obtained from a healthy or non-diseased individual, or a healthy or non-diseased sample obtained from the same individual from whom the test sample is obtained.

In certain embodiments, the IL-36R related disease, disorder or condition include, but are not limited to, inflammatory diseases, autoimmune diseases, respiratory diseases, metabolic disorders, and cancer.

In certain embodiments, the inflammatory disease is selected from the group consisting of allergic inflammation of the skin, lungs, and gastrointestinal tract, atopic dermatitis (also known as atopic eczema), asthma (allergic and non-allergic), and epithelial-mediated inflammation. In some embodiments, the autoimmune disease is selected from the group consisting of psoriasis vulgaris (commonly referred to as psoriasis), hidradenitis suppurativa, generalized pustular psoriasis (GPP), palmo-plantar pustulosis (PPP), inflammatory bowel disease, atopic dermatitis and Acne Vulgaris. In some embodiments, the respiratory disease is selected from the group consisting of asthma, cystic fibrosis, emphysema, chronic obstructive pulmonary disease (COPD), and acute respiratory distress syndrome. In some embodiments, the metabolic disease is selected from the group consisting of obesity, type 2 diabetes, atherosclerosis, and cardiovascular disease. In some embodiments, the cancer can be any type of cancer known in the art, including but not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, leukemia, lymphoma, and Merkel cell carcinoma.

In some embodiments, the disease or disorder is selected from the group consisting of psoriasis, pustular psoriasis, hidradenitis suppurativa, ichthyosis, inflammatory bowel disease (IBD), atopic dermatitis, Acne Vulgaris, and Behcet's disease.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development.

Dosages may be proportionally reduced or increased by a person skilled in the art (e.g., physician or veterinarian) as indicated by these and other circumstances or requirements.

In certain embodiments, the antibody or antigen-binding fragment provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies or antigen-binding fragments thereof provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments thereof provided herein may be administered alone or in combination a therapeutically effective amount of a second therapeutic agent. For example, the antibodies or antigen-binding fragments thereof disclosed herein may be administered in combination with a second therapeutic agent.

In some embodiments, the second therapeutic agent can be those which are useful for treating psoriasis, e.g. GPP (generalized pustular psoriasis), PPP (palmoplantar pustulosis), HS (hidradenitis suppurativa), Behcet's disease, and the like.

In some embodiments, the second therapeutic agent is a hormone-based immunosuppressant. In some embodiments, the second therapeutic agent comprises glucocorticoids or steroids. Non-limiting exemplary glucocorticoids or steroids include budesonide, flunisolide, triamcinolone acetonide, fluticasone propionate, beclomethasone diproprionate, and ciclesonide.

In some embodiments, the second therapeutic agent is a therapeutic agent or drug for psoriasis. In some embodiments, the second therapeutic agent can be a non-biologic agent, such as methotrexate, ciclosporin, fumaric acid esters (FAE), hydroxycarbamide, fumarates (such as dimethyl fumarate), retinoids (synthetic forms of vitamin A), glucocorticoids or steroids. In some embodiments, the second therapeutic agent can be a biologic agent, that interrupt the immune process involved in psoriasis, targeting specific aspects of the immune system contributing to psoriasis. In some embodiments, the biologic agent is a monoclonal antibody targeting anti-IL17, anti-IL12/23, anti-IL23, and/or anti-TNF alpha, etc. Examples of such monoclonal antibody include but are not limited to infliximab, adalimumab, golimumab, certolizumab pegol, ixekizumab, ustekinumab, guselkumab, efalizumab, alefacept, secukinumab, and brodalumab.

In some embodiments, the second therapeutic agent is a therapeutic agent or drug for GPP (generalized pustular psoriasis). In some embodiments, the second therapeutic agent comprises a therapeutic agent or a drug such as glucocorticoid or steroid, etanercept, PUVA, hydroxyurea, dapsone, cyclosporin A, adalimumab, etretinate, isotretinoin, acitretin.

In some embodiments, the second therapeutic agent is a therapeutic agent or drug for PPP (palmoplantar pustulosis). In some embodiments, the second therapeutic agent comprises a therapeutic agent or a drug such as a retinoid, ciclosporin, tetracycline, colchicine, Tripterygium wilfordii, Tripterygium hypoglaucum hutch, an anti-interleukin 23 monoclonal antibody (such as guselkumab).

In some embodiments, the second therapeutic agent is a therapeutic agent or drug for HS (hidradenitis suppurativa). In some embodiments, the second therapeutic agent comprises a therapeutic agent or a drug such as corticosteroid, an antibiotic, an anti-androgenic agent, an anti-inflammatory agent. Examples of antibiotics include but are not limited to rifampicin, clindamycin, tetracycline and minocycline. Examples of anti-androgenic agent include but are not limited to spironolactone, flutamide, cyproterone acetate, ethinylestradiol, finasteride, dutasteride, and metformin. Examples of anti-inflammatory agent include but are not limited to a TNF inhibitor, such as infliximab, etanercept and adalimumab.

In some embodiments, the second therapeutic agent is a therapeutic agent or drug for Behcet's disease. In some embodiments, the second therapeutic agent comprises a therapeutic agent or a drug such as topical drugs, corticosteroids, methotrexate, colchicine, thalidomide, azathioprine, cyclophosphamide, cyclosporine, mycophenolate mofetil and anti-tumor necrosis factor antagonists. Examples of anti-tumor necrosis factor antagonist include but are not limited to infliximab, etanercept and apremilast.

In some embodiments, the second therapeutic agent is a drug targeting IL-17, IL-23, TNF, IL-12, IL-1, etc.

In certain of these embodiments, an antibody or antigen-binding fragment thereof provided herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition. However, an antibody or antigen-binding fragment thereof administered “in combination” with another therapeutic agent does not have to be administered simultaneously with or in the same composition as the agent. An antibody or antigen-binding fragment thereof administered prior to or after another agent is considered to be administered “in combination” with that agent as the phrase is used herein, even if the antibody or antigen-binding fragment and the second agent are administered via different routes. Where possible, additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57^{th} edition (November 2002)) or protocols well known in the art.

In another aspect, the present disclosure further provides methods of modulating IL-36R activity in IL-36R-positive cells, comprising exposing the IL-36R-positive cells to the antibodies or antigen-binding fragments thereof provided herein.

In another aspect, the present disclosure provides methods of detecting the presence or amount of IL-36R in a sample, comprising contacting the sample with the antibody or antigen-binding fragment thereof provided herein, and determining the presence or the amount of IL-36R in the sample.

In another aspect, the present disclosure provides a method of diagnosing an IL-36R related disease, disorder or condition in a subject, comprising a) contacting a sample obtained from the subject with the antibody or an antigen-binding fragment thereof provided herein; b) determining the presence or amount of IL-36R in the sample; and c) correlating the presence or the amount of IL-36R to existence or status of the IL-36R related disease, disorder or condition in the subject.

In another aspect, the present disclosure provides kits comprising the antibody or antigen-binding fragment thereof provided herein, optionally conjugated with a detectable moiety, which is useful in detecting an IL-36R related disease, disorder or condition. The kits may further comprise instructions for use.

In another aspect, the present disclosure also provides use of the antibody or antigen-binding fragment thereof provided herein in the manufacture of a medicament for treating, preventing or alleviating an IL-36R related disease, disorder or condition in a subject, in the manufacture of a diagnostic reagent for diagnosing an IL-36R related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Antibody Generation

### 1.1 Immunization and Antibody Screening

Mice from various strains (C57BL/6, BALB/c, SJL and CD-1) were immunized with recombinant human IL-36R-his protein (extracellular domain, from 1^{st} to 337^{th} amino acid residue of SEQ ID NO: 211). Those which generated a strong titer response were selected for single B cell isolation. B cells from spleen and lymph nodes were isolated and enriched with microbeads. The B cells which recognize hIL-36R were stained and isolated by FACS. The immunoglobin heavy chain and light chain sequences of these B cells were cloned and recombinantly expressed. These monoclonal antibodies then were taken into rescreening for hIL-36R binding and signaling blocking. The variable region sequences of selected anti-IL-36R antibodies were listed in Tables 1 and 2 in the present disclosure.

### 1.2 Expression of Benchmark Antibodies

IL36R antibodies BI655130, ANB019 and REGN14 was used as benchmark. Sequences of benchmark antibodies were acquired from patents: U.S. Ser. No. 10/550,189, US20200017592A1, U.S. Ser. No. 10/526,410B2 or IMGT information system (code 10845). The coding DNA sequences were synthesized and cloned into pcDNA3.1 vector to construct antibody expression plasmids. The expression plasmids were transfected into CHO-s cells with ExpiCHO™ Expression System (Gibco, A29133). After 14 days, the supernatants were collected, and the antibodies were purified by affinity chromatography with a protein A column.

Variable sequences of benchmark antibodies were listed as below:

VH of BI655130 (SEQ ID NO: 205):
QVQLVQSGAEVKKPGASVKVSCKASGYSFTSSWIHWVKQAPGQGLE
WMGEINPGNVRTNYNENFRNKVTMTVDTSISTAYMELSRLRSDDTAVYY
CTVVFYGEPYFPYWGQGTLVTVSS
VL of BI655130 (SEQ ID NO: 206):
QIVLTQSPGTLSLSPGERATMTCTASSSVSSSYFHWYQQKPGQAPRL
WIYRTSRLASGVPDRFSGSGSGTDFTLTISRLEPEDAATYYCHQFHRSP
LTFGAGTKLEIK
VH of ANB019 (SEQ ID NO: 207):
QVQLVQSGAEVKKPGASVKVSCKASGYTFTNYWMNWVRQAPRQG
LEWMGMFHPTGDVTRLNQKFKDRVTMTRDTSTSTVYMELSSLRSEDTAV
YYCARTTSMIIGGFAYWGQGTLVTVSS
VL of ANB019 (SEQ ID NO: 208):
DIVMTQTPLSLSVTPGQPASISCRSSKSLLHRNAITYFYWYLHKPGQP
PQLLIYQMSNLASGVPDRFSGSGSGTDFTLKISRVEAEDVGVYYCAQN
LELPLTFGGGTKVEIK
VH of REGN14 (SEQ ID NO: 209):
EVQLVESGGDLVQPGRSLRLSCAASGFTFDDYAMHWVRQAPGKGLE
WVSVISWNSDVIAYSDSVKGRFTISRDNAKNSLYLQMNSLRTEDTALYY
CTKGHKWSFFDYWGQGTLVTVSS
VL of REGN14 (SEQ ID NO: 210):
EIVLTQSPATLSLSPGERATLSCRASQSISSYLAWYQQKPGQAPRLLIF
NVANRATDIPARFSGSGSGTDFTLTISSLEPEDFAVYYCQQRSNWPLTF
GGGTKVEIK

### Example 2. IL-36R Binding Test by Biolayer Interferometry (BLI)

Antibodies identified in Example 1 were diluted with kinetics buffer (PBS pH 7.4, 0.1% BSA+0.01% Tween-20) to a concentration of 100 nM. Recombinant hIL-36R protein (Acrobio, Cat #IL2-H52H6) was diluted with kinetics buffer to get a serials of concentration gradients: 500 nM, 250 nM, 125 nM and 0 nM as reference control well. Antibodies was immobilized onto Protein A biosensor after balance. A baseline was detected for 60 seconds. And then antibody-antigen association was detected for 180 seconds to get the Kon factor data. Followed by dissociation in kinetic buffer for 180 seconds to get the Koff factor data. The regeneration of biosensors was in buffer 10 mM glycine, pH2.0. All the kinetics data were collected at 30° C. Data were acquired with Gator Bioanalysis System.

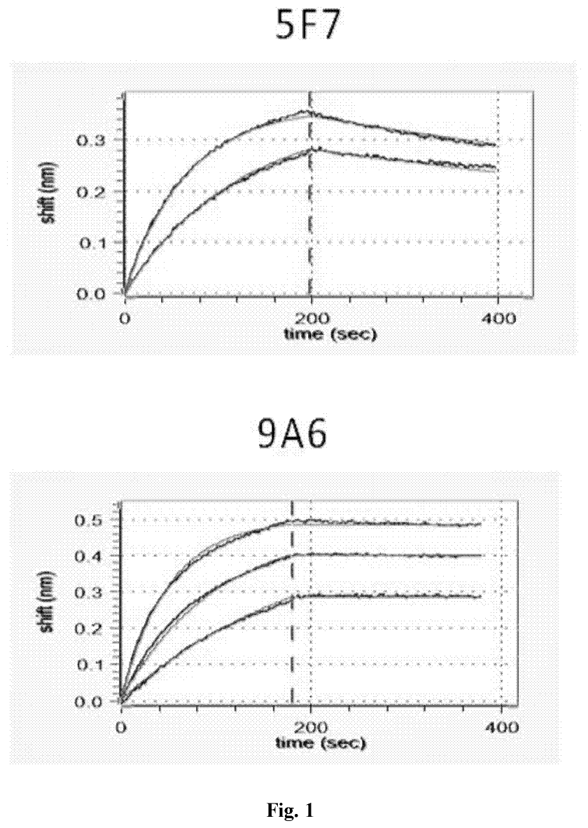

As shown in FIG. 1 and Table 6, chimeric antibodies 5F7 and 9A6 bind hIL-36R with a KD value 1.46E-08 M and 2.05E-11 M, respectively.

TABLE 6
Binding affinity of IL-36R antibodies

| clone | kon(1/s) | koff(1/Ms) | KD(M) |
|---|---|---|---|
| 5F7 | 5.91E+04 | 0.000861 | 1.46E−08 |
| 9A6 | 3.97E+04 | 8.14E−07 | 2.05E−11 |
| 9C12 | 0.000795 | 7.94E+04 | 1.00E−08 |
| 10D12 | 0.000321 | 7.98E+04 | 4.03E−09 |
| 10F6 | 0.000137 | 1.52E+05 | 9.01E−10 |
| 9H11 | 5.79E−07 | 9.17E+04 | 6.31E−12 |
| 1C11 | 0.000872 | 6.38E+04 | 1.37E−08 |
| 1A21 | 4.01E−05 | 7.49E+04 | 5.36E−10 |
| 1J3 | 4.37E−05 | 7.32E+04 | 5.97E−10 |
| 1J22 | 0.000747 | 6.80E+04 | 1.10E−08 |

A reporter cell line of Jurkat-IL36R-IL1Racp-NFkB-luc cells was used to evaluate the blocking activity of anti-IL-36R antibodies of Table 1.

As shown in FIG. 18, 5F7 and 9A6 show the highest blocking activity among all anti-IL-36R antibodies of Table 1.

### Example 3. Blocking Activity Evaluation by IL36R Reporter Assay

A reporter cell line, Jurkat-IL36R-IL1Racp-NFkB-luc cells, was used to evaluate the blocking activity of anti-IL-36R antibodies. Jurkat-IL36R-IL1Racp-NFkB-luc cells were collected and seeded into 96 well white plate at a concentration of 50000 cells/25 ul per well. 50 ul serially diluted antibodies were added and mixed with the cells and incubated at 37° C. for 30 min. Then 25 ul/well 2 ng/ml IL-36 α ligands were added and mixed with cells then incubated at 37° C. for 4.5h. After that, 50 ul/well One-Glu reagent was added into the plate and the luminescence signal was read with Tecan Spark.

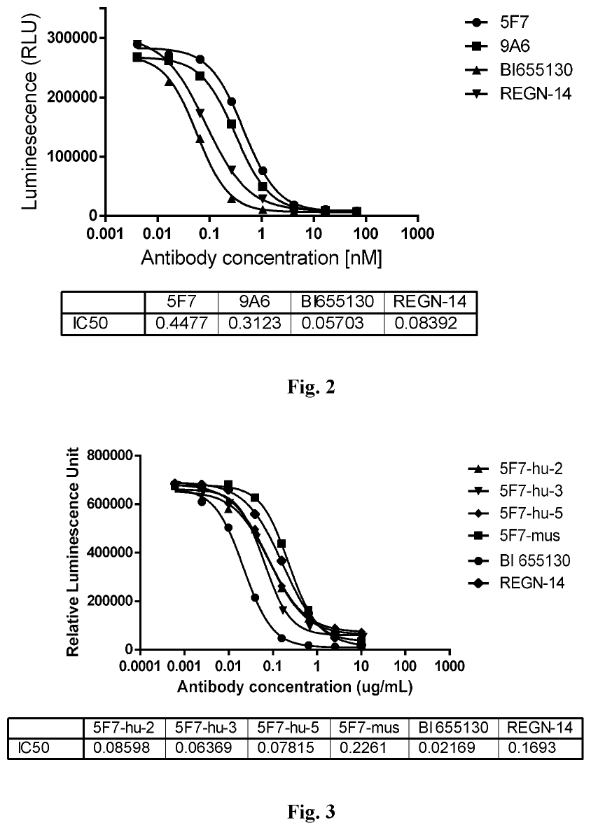

As shown in FIG. 2, chimeric antibodies 5F7 and 9A6 blocked the ligand IL-36a induced IL-36R signaling with an IC₅₀ 0.4477 nM and 0.3123 nM, respectively.

### Example 4. Antibody Humanization

The antibody clone 5F7 was chosen for humanization. Firstly, IgBLAST from NCBI was used to choose the most appropriate human frameworks to graft rodent CDRs. Variable regions with high amino acid sequence identity to the rodent variable regions (homology matching or best fit) were used. Secondly, 5F7 mAb was humanized by grafting the three CDRs from the light chain variable region into a human VL that was as homologous as possible to the mouse antibody VL. Similarly, their three CDRs from the heavy chain variable region were grafted into a human VH that was as homologous as possible to the mouse antibody. Furthermore, in the framework region of the selected human variable regions, a few amino acid residues were changed into the amino acid residues that were present in the mouse variable regions (the so-called back mutations). Humanized sequences of 5F7 were listed in Tables 3-5 of the present disclosure. Activity of humanized 5F7 were evaluated by BLI and IL-36R reporter assay. Experiment procedures are as described in Examples 2 and 3.

As shown in Table 7 and FIG. 3, the binding and blocking activity of 5F7 original molecular was reproduced after humanization.

TABLE 7
Binding affinity of humanized 5F7

| antibody | koff(1/s) | kon(1/Ms) | KD(M) |
|---|---|---|---|
| 5F7-hu-2 | 0.000233 | 3.40E+04 | 6.86E−09 |
| 5F7-hu-3 | 0.000194 | 4.27E+04 | 4.55E−09 |
| 5F7-hu-5 | 0.000294 | 4.67E+04 | 6.28E−09 |

### Example 5. Antibody Affinity Maturation

To enhance the binding affinity of the humanized 5F7 antibodies, a combined CDR1 walking randomization and rational design-based approach was used for affinity maturation. Firstly, mutations were introduced in CDR-H1 and CDR-L1 by overlap PCR method which using oligonucleotides containing the NNK codon at the mutation sites, and prior to selection, DNA sequencing was performed to confirm that the clones from the library were not biased. Secondly, the selection experiments were performed three times independently at different concentrations of 2 μg/mL, 1 μg/mL and 0.5 μg/mL of soluble IL-36R-his. After three rounds of selection, the amino acid sequences of clones randomly selected from last round were analyzed, then the enriched clones from 0.5 μg/mL soluble IL-36R-his concentrations were obtained. Then one light chain and 7 heavy chains of the enriched clones above were cloned into pCDNA3.1+ with hIgG4 constant region sequence and expressed in CHOs cells. Wherein, the heavy chain constant region was with an S228P mutation. Variable region sequences of these clones were listed in Table 4 of the present disclosure. The binding and blocking activity of these clones were evaluated by Gator Bioanalysis System and IL-36R reporter assay.

Amino acid sequence of heavy chain constant
region (SEQ ID NO: 203):
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKR
VESKYGPPCPPCPAPEFLGGPSVFLFPPKPKDTLMISRTPEVTCVVVDV
SQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVD
KSRWQEGNVFSCSVMHEALHNHYTQKSLSLSLGK
Amino acid sequence of light chain constant
region (SEQ ID NO: 202):
RTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSS
PVTKSFNRGEC

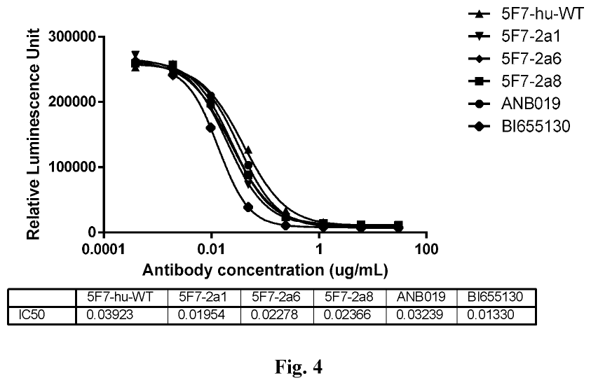

As shown in Table 8 and FIG. 4, 5F7 mutants with higher affinity were successfully isolated. And the blocking activity of 5F7 was also elevated after maturation.

TABLE 8
Binding affinity of 5F7 after maturation (tested by BLI)

| Clone | Kon (1/Ms) | Koff (1/s) | KD (M) |
|---|---|---|---|
| 5F7-2a1 | 3.25E+04 | NA | <1E−10 |
| 5F7-2a6 | 3.98E+04 | NA | <1E−10 |
| 5F7-2a8 | 4.71E+04 | NA | <1E−10 |
| 5F7-2c4 | 203 | 0.000418 | 2.06E−06 |
| 5F7-2d3 | 3.59E+04 | 8.83E−05 | 2.46E−09 |
| 5F7-2g10 | 2.89E+04 | 1.84E−05 | 6.38E−10 |
| 5F7-2h1 | 4.10E+04 | NA | <1E−10 |

### Example 6. Binding Affinity Test by SPR

To evaluate binding affinity of antibodies to hIL-36R, the binding affinity of antibodies were tested by surface plasmon resonance (SPR) technology with Biacore T200. The assay was performed at 25° C. and the running buffer was HBS-EP+. Diluted antibodies were captured on the sensor chip through Fc capture method. Reference antibodies BI655130, ANB019, and REGN14 were made by recombinant methods. The heavy chain variable sequence and light chain variable sequence are set forth in SEQ ID Nos 205 and 206, respectively, for BI655130; and are set forth in SEQ ID Nos 207 and 208, respectively, for ANB019; set forth in SEQ ID Nos 209 and 210, respectively, for REGN14. hIL-36R was used as the analyte, followed by injecting running buffer as dissociation phase.

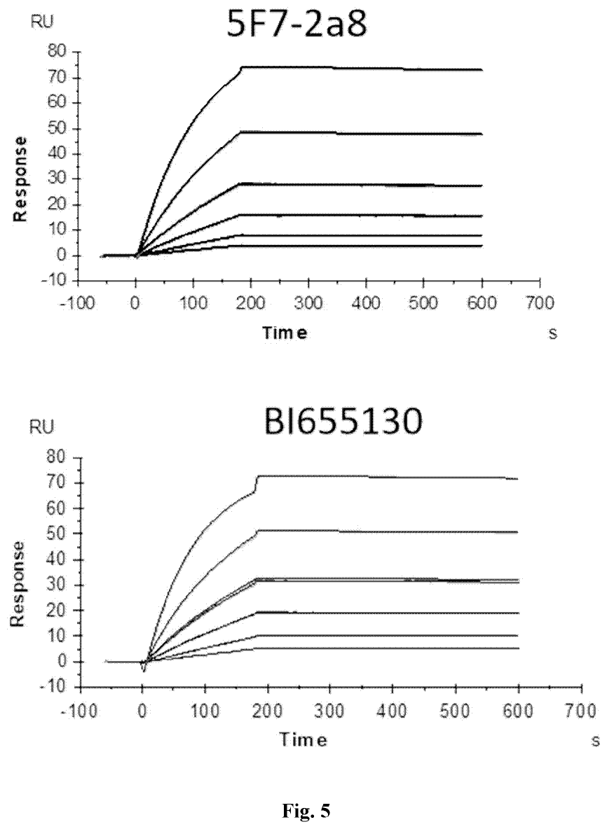

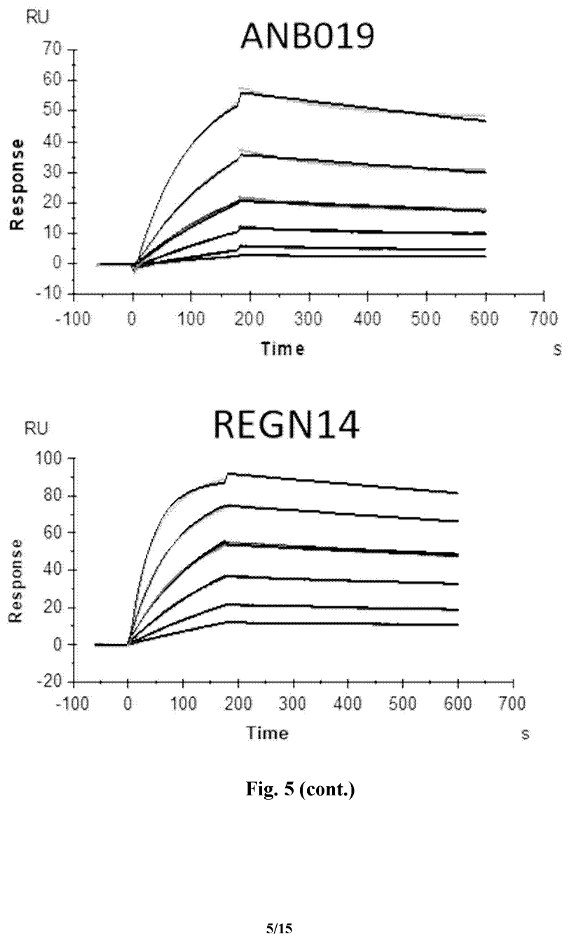

As shown in FIG. 5 and Table 9, 5F7-2a8 binds with hIL-36R with a K_{D} value of 1.52×10⁻⁹ M.

TABLE 9
Binding affinity of antibodies to hIL-36R (tested by SPR)

| | | | | Rmax | Chi2 |
|---|---|---|---|---|---|
| Antibody | ka (1/Ms) | kd (1/s) | KD (M) | (RU) | (RU2) |
| 5F7-2a8 | 2.95E+04 | 4.48E−05 | 1.52E−09 | 91.62 | 0.0256 |
| BI655130 | 3.92E+04 | 3.66E−05 | 9.35E−10 | 81.75 | 0.0518 |
| ANB019 | 3.35E+04 | 4.44E−04 | 1.32E−08 | 68.13 | 0.178 |
| REGN14 | 7.56E+04 | 2.83E−04 | 3.74E−09 | 93.92 | 0.241 |

### Example 7. Antibody-hIL36R Binding Test by ELISA

Recombinant hIL36R protein was coated at a concentration of 1 μg/ml 100 ul per well in ELISA plate overnight. After wash, the plate was blocked with PBS containing 1% BSA+1% normal goat serum+0.05% Tween20. After that, 100 ul serially diluted antibodies were added into the plate and incubated for 1 h. The specific binding to hIL36R of antibodies was detected by HRP linked anti-hIgG antibody.

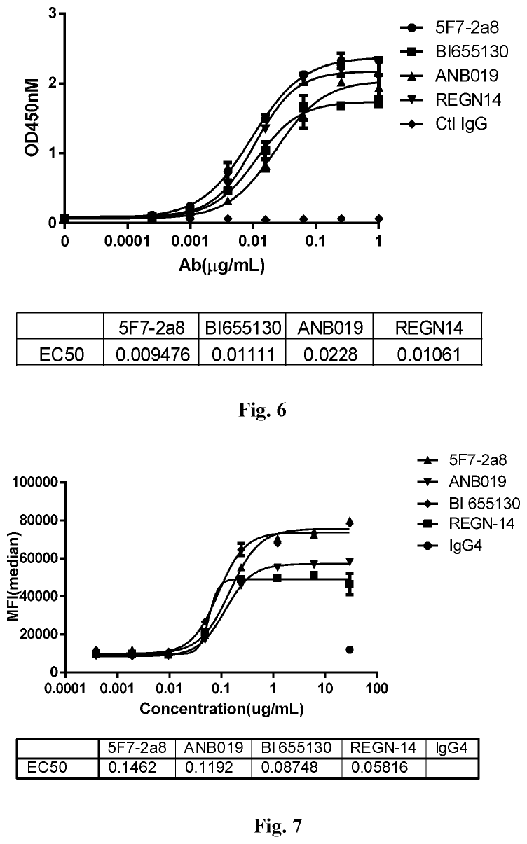

As shown in FIG. 6, 5F7-2a8 binds recombinant hIL-36R-his with an EC₅₀ of 0.00947 ug/ml.

### Example 8. Binding to Membrane IL-36R

To evaluate the binding ability of antibodies to cell membrane IL-36R of various species, HEK293 cells were transfected with pcDNA3.1-hIL36R, pcDNA3.1-cynoIL36R or pcDNA3.1-musIL36R plasmids with Lipofectamine2000 (Invitrogen, Cat #11668-019). After 48 h, the transfected cells were subjected to FACS analysis for anti-IL-36R antibodies binding. Cells were collected and seeded into 96 well plate at a concentration of 100000 cells/50 ul/well. 50 ul serially diluted antibodies were added to the cells and incubate at 4° C. for 1 h. After that, cells were washed twice with PBS and 100 ul diluted secondary antibody (FITC labeled anti-hIgG) was added into the cells and incubated at 4° C. for 30 min. After incubation, cells were washed twice with PBS and analyzed by flow cytometry.

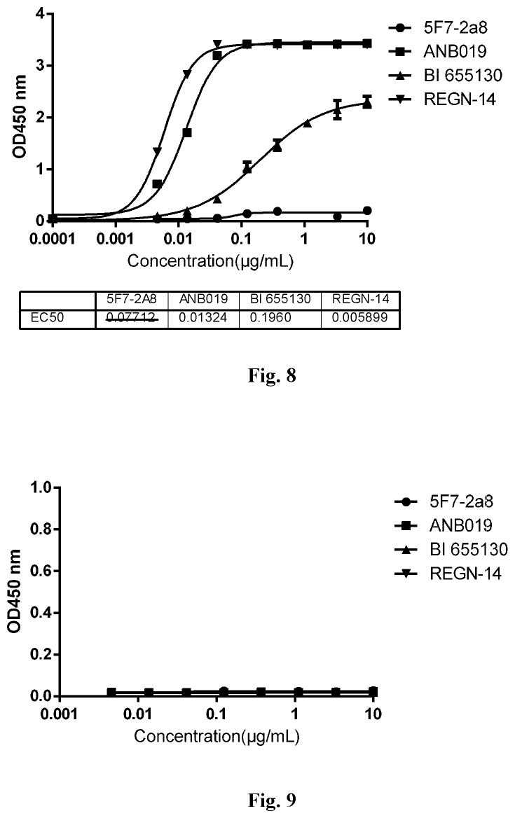

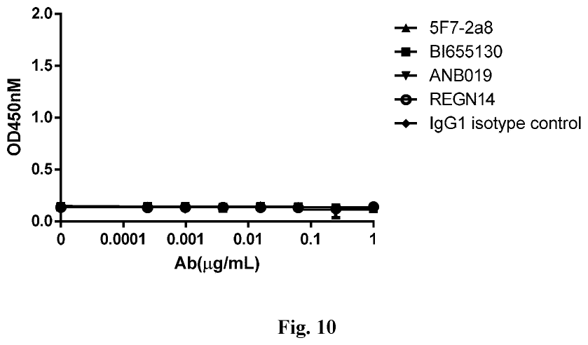

As shown in FIG. 7, 5F7-2a8 binds with membrane hIL-36R with an EC50 of 0.1462 ug/ml. As shown in FIG. 8 and FIG. 9, 5F7-2a8 has no cross reactivity with cynomolgus or mouse IL-36R.

### Example 9. Cross Reactivity Test to Cynomolgus and Mouse IL-36R

To test whether 5F7 bind to recombinant cyno/mouse IL-36R protein or not, antibodies were diluted with kinetics buffer (PBS pH 7.4, 0.1% BSA+0.01% Tween-20) to a concentration of 100 nM. Recombinant cynomolgus IL36R protein (Acrobio, Cat #IL2-C52H5) or mouse IL-36R (R&D, Cat #2354-RP) was diluted with kinetics buffer to get a serials of concentration gradients: 100 nM, 50 nM, 25 nM, 12.5 nM and 0 nM as reference control well. Antibodies was immobilized onto Protein A biosensor after balance. Cyno or mouse IL-36R was used as the analyte, followed by injecting running buffer as dissociation phase.

As shown in Table 10, 5F7-hu-3 has no cross reactivity with cynomolgus or mouse IL-36R.

TABLE 10
Cross reactivity to cynomolgus and mouse IL-36R

| | KD (M) | |
|---|---|---|
| Antibody | cynomolgus | mouse |
| 5F7-hu-3 | No binding | No binding |
| BI655130 | >1E−05M | No binding |
| REGN1412 | 7.26E−09M | No binding |

### Example 10. Binding Specificity

To test the binding specificity of 5F7-2a8, IL1R1 is the most homology related IL-1R family member to IL-36R. Recombinant hIL1R1 protein was used to test whether 5F7-2a8 bind with IL1R1 or not. hIL1R1 protein was coated at a concentration of 1 g/ml 100 ul per well in ELISA plate overnight. Then the plate was blocked with PBS containing 1% BSA+1% normal goat serum+0.05% Tween20. After that, 100 ul serially diluted antibodies were added into the plate and incubated for 1 h. The specific binding to hIL1R1 of antibodies was detected with HRP linked anti hIgG antibody.

As shown in FIG. 10, 5F7-2a8 has no binding activity with hIL1R1.

### Example 11. Antibody Block IL-36α/β/γ Induced IL-8 Release in A431 Cells

To evaluate the activity of anti-IL-36R antibody to inhibit IL-8 release in A431 cells, A431 cells were collected and seeded into 96 well plate at a concentration of 50000 cells/50 ul/well. 100 ul serially diluted antibodies were added to the cells and incubate at 37° C. for 30 min. After that, 50 ul 300 ng/ml IL-36α or 100 ng/mL IL-36β or 100 ng/mL IL-36γ was added into the plate and incubated at 37° C. for 24 h. After incubation, supernatants were transferred to 96 well V-bottom plate and centrifuged at 1500 g for 5 min. The cell free supernatants were carefully collected and stored at −70° C. freezer. The IL-8 concentration in the supernatants were detected with IL-8 ELSIA kit (DAKEWE, Cat #1110802).

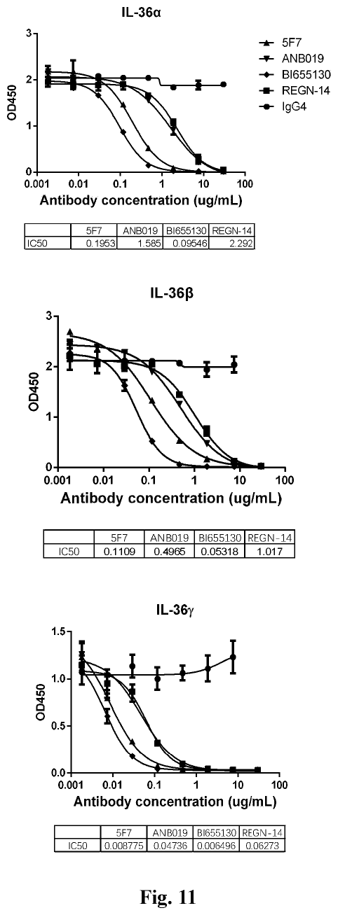

As shown in FIG. 11, 5F7-2a8 blocks IL-36α, IL-36β or IL-36γ induced IL-8 release in A431 cells with an IC50 of 0.1953, 0.1109, and 0.008775 ug/ml respectively. It can be seen that 5F7-2a8 potently inhibit the IL-36α induced IL-8 production in A431 cells. The inhibition activity of 5F7-2a8 is comparable to BI655130, but is much more potent than that of ABN019 or REGN14.

### Example 12. Antibody Block IL-36α Induced IL-8 Release in HDF Cells

To evaluate the activity of anti-IL-36R antibody to inhibit IL-8 release in primary human dermal fibroblast (HDF) cells, HDF cells were collected and seeded into 96 well plate at a concentration of 5000 cells/50 ul/well. 100 ul serially diluted antibodies were added to the plate and incubate at 37° C. for 30 min. After that, 50 ul 300 ng/ml IL-36α was added into the cells and incubated at 37° C. for 24 h. After incubation, supernatants were transferred to 96 well V-bottom plate and centrifuged at 1500 g for 5 min. The cell free supernatants were carefully collected and stored at −70° C. freezer. The IL-8 concentration in the supernatants were detected with IL-8 ELSIA kit (DAKEWE, Cat #1110802).

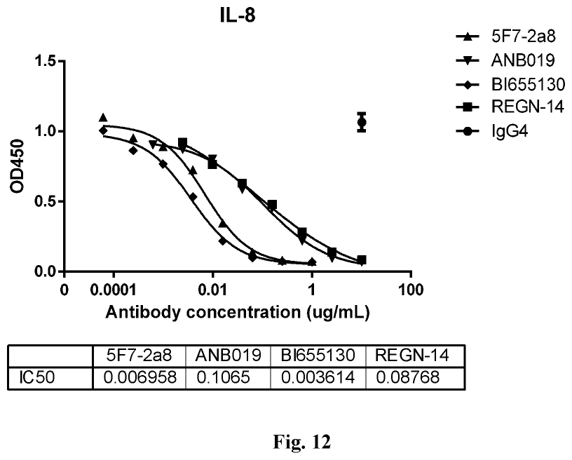

As shown in FIG. 12, 5F7-2a8 blocks IL-36α induced IL-8 release in HDF cells with an IC50 of 0.006958 ug/ml. It can be seen that 5F7-2a8 potently inhibit the IL-36α induced IL-8 production in HDF cells. The inhibition activity of 5F7-2a8 is comparable to BI655130, but is much more potent than that of ABN019 or REGN14.

### Example 13. Antibody Block IL-36α/β/γ Induced IL-8/IL-6/TNF-a Release in HEK Cells

To evaluate the activity of anti-IL-36R antibody to inhibit IL-8 release in primary human epithelial keratinocytes (HEK) cells, procedure of stimulation and blocking of HEK cells as described in part 11. Concentrations of IL-8, IL-6 and TNF-a were detected with Multi-Analyte Flow Assay Kit (Biolegend) as manufacture's instruction.

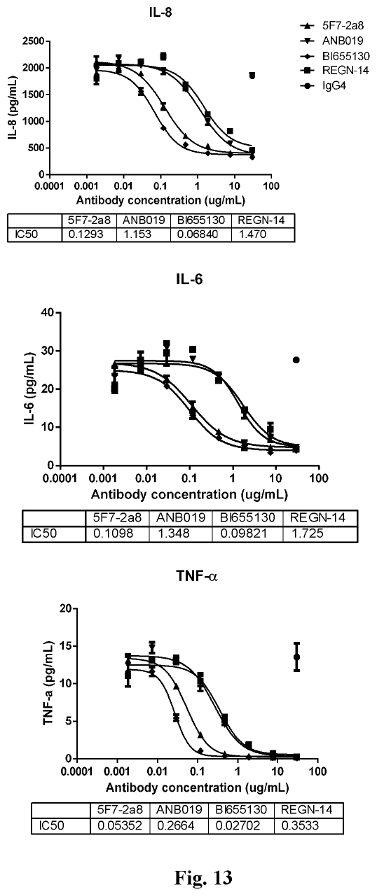

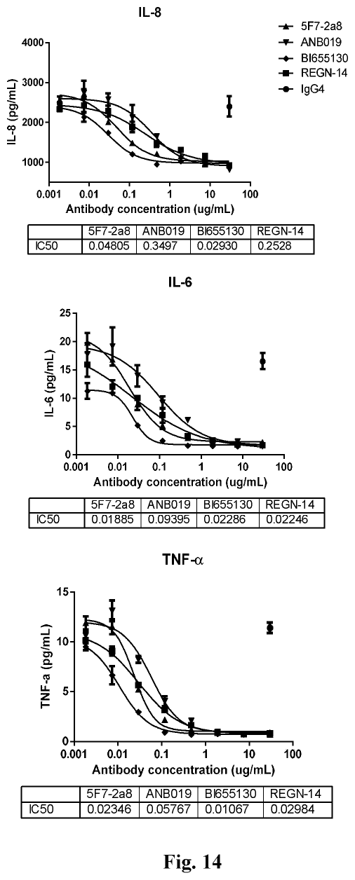

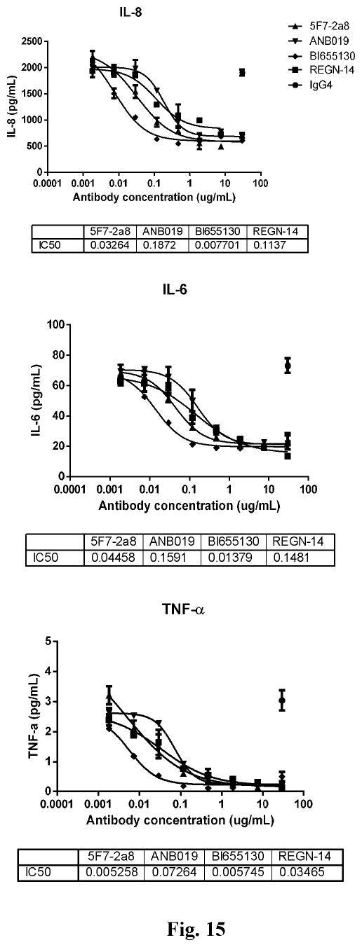

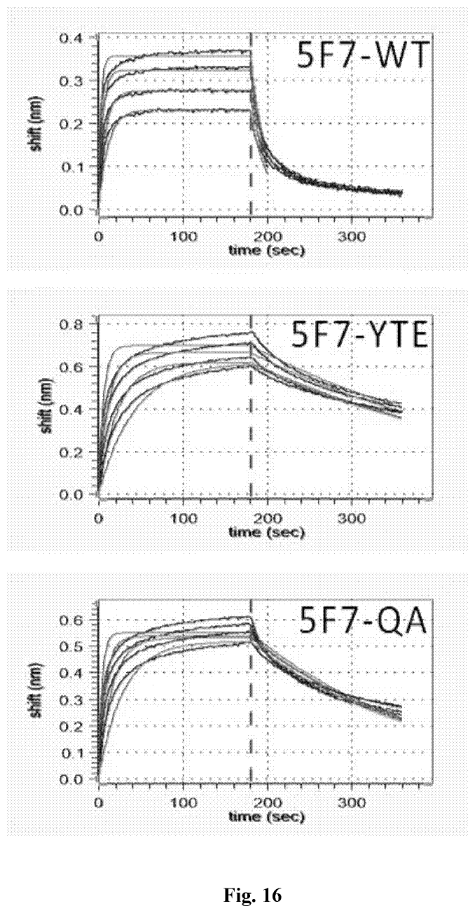

As shown in FIG. 13, 5F7-2a8 blocks IL-36α induced IL-8/IL-6/TNF-a release in HEK cells with an IC50 of 0.1293, 0.1098 and 0.05352 ug/ml, respectively. As shown in FIG. 15, 5F7-2a8 blocks IL-36β induced IL-8/IL-6/TNF-a release in HEK cells with an IC50 of 0.04805, 0.01885 and 0.02346 ug/ml, respectively. As shown in FIG. 16, 5F7-2a8 blocks IL-36γ induced IL-8/IL-6/TNF-a release in HEK cells with an IC50 of 0.03264, 0.04458 and 0.005258 ug/ml, respectively.

It can be seen that 5F7-2a8 potently inhibit the IL-36 (a//) induced IL-8, IL-6, and TNFα production in HEK cells. The inhibition activity of 5F7-2a8 is comparable to BI655130, but is much more potent than that of ABN019 or REGN14.

### Example 14. Fc Mutations to Pro-Long Antibody Half Life

To pro-long the half-life of 5F7-2a8, mutations that enhance antibody-FcRn binding were introduced into IgG4 Fc domain. M252Y/S254T/T256E (YTE) mutations were introduced in 5F7-2a8-YTE molecule, and T307Q/N434A (QA) mutations were introduced into 5F7-2a8-QA molecule. The heavy chain constant region sequence of 5F7-2a8-WT with wild type Fc was showed as SEQ ID NO: 203. The heavy chain constant region of 5F7-2a8-YTE was showed as SEQ ID NO: 201. The heavy chain constant region of 5F7-2a8-QA was showed as SEQ ID NO: 204.

Amino acid sequence of heavy chain constant
region of 5F7-2a8-YTE (SEQ ID NO: 201):
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKR
VESKYGPPCPPCPAPEFLGGPSVFLFPPKPKDTLYITREPEVTCVVVDV
SQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVD
KSRWQEGNVFSCSVMHEALHNHYTQKSLSLSLGK
Amino acid sequence of heavy chain constant
region of 5F7-2a8-QA (SEQ ID NO: 204):
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKR
VESKYGPPCPPCPAPEFLGGPSVFLFPPKPKDTLMISRTPEVTCVVVDV
SQEDPEVQFNWYVDGVEVHNAKTKPREEQFNSTYRVVSVLQVLHQDWLN
GKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVD
KSRWQEGNVFSCSVMHEALHAHYTQKSLSLSLGK.

The light chain constant region sequence of 5F7-2a8-WT, 5F7-2a8-YTE and 5F7-2a8-QA are identical as SEQ ID NO: 202. The binding affinity of antibodies to hFcRn was evaluated with Gator Bio-analysis system. Briefly, recombinant hFcRn-his/B2M heterodimer protein (Acrobio, Cat #FCM-H82W4) was loaded to anti-his sensor, serially diluted antibodies were used as analyte. Association and dissociation were performed in pH6.0 KD buffer.

As shown in FIG. 16 and Table 11, the binding affinity of 5F7-2a8-YTE and 5F7-2a8-QA were increased about 10 times compared with 5F7-2a8-WT.

TABLE 11
Antibody binding affinity to hFcRn

| | koff(1/s) | kon(1/Ms) | KD(M) |
|---|---|---|---|
| 5F7-WT | 0.0507 | 3.01E+05 | 1.68E−07 |
| 5F7-YTE | 0.00277 | 1.80E+05 | 1.54E−08 |
| 5F7-QA | 0.00398 | 2.90E+05 | 1.37E−08 |

### Example 15. PK Analysis of 5F7-2a8-YTE and 5F7-2a8-QA

To evaluate the pharmacokinetics of 5F7-2a8-YTE and 5F7-2a8-QA in cynomolgus and hFcRn transgenic mouse, for PK test in cynomolgus, 2 animals (1 male and 1 female) in each group were administrated by single dose i.e. infusion at a dosage of 5 mg/kg. Serum was collected at the following time points after drug administration: 0 h, 0.5h, 2 h, 8h, 24 h, 48h, 3d, 4d, 7d, 14d, 21d, 28d, 35d, 42d, 49d. Serum antibody concentrations were detected by ELISA. Briefly, recombinant hIL36R protein was coated at a concentration of 1 μg/ml 100 ul per well in ELISA plate overnight. After wash, the plate was blocked with PBS containing 1% BSA+1% normal goat serum+0.05% Tween20. After that, 100 ul appropriately diluted serums or certain concentrations of antibodies were added into the plate and incubated for 1 h. Then the binding of antibodies was detected with HRP linked anti-hIgG antibody. The serum drug concentrations were calculated with standard curve that from certain concentrations samples.

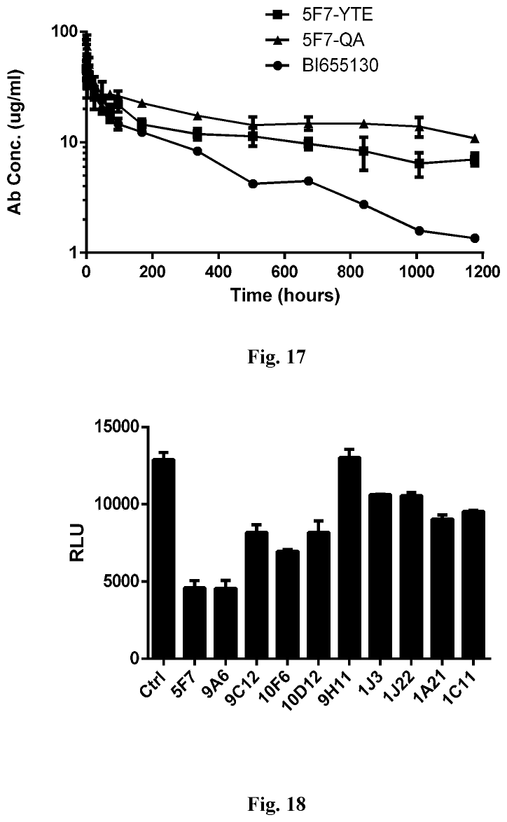

As shown in FIG. 17, Table 12 and Table 13, half-life of 5F7-2a8-YTE and 5F7-2a8-QA were significantly pro-longed in cynomolgus monkey compared with benchmark antibody which Fc without FcRn binding enhancement.

TABLE 12
PK test results in cynomolgus monkey

| | | 5F7-YTE | | | 5F7-QA | | | BI655130 | | |
|---|---|---|---|---|---|---|---|---|---|---|
| PK Parameter | unit | Cyno 1 | Cyno 2 | Mean | Cyno 1 | Cyno 2 | Mean | Cyno 1 | Cyno 2 | Mean |
| t½ | h | 1114.534 | 684.215 | 754.876 | 623.478 | 885.760 | 787.077 | 273.261 | 104.728 | 187.786 |
| Tmax | h | 0.5 | 0 | 0 | 0 | 2 | 2 | 0.5 | 2 | 0.5 |
| Cmax | μg/ml | 44.584 | 52.428 | 46.513 | 77.444 | 88.024 | 74.625 | 46.302 | 57.364 | 48.641 |
| AUC 0-t | μg/ml*h | 11813.450 | 10669.839 | 11241.644 | 15385.166 | 16535.243 | 15960.204 | 7542.024 | 3183.386 | 5879.557 |
| AUC 0-inf_obs | μg/ml*h | 28388.416 | 16963.178 | 20326.408 | 28466.653 | 35736.380 | 32748.187 | 8627.248 | 5042.706 | 6252.442 |
| MRT 0-inf_obs | h | 1577.719 | 876.614 | 1048.786 | 984.435 | 1303.732 | 1183.625 | 378.998 | 158.061 | 250.799 |
| Cl_obs | ml/day/kg | 4.227 | 7.074 | 5.904 | 4.215 | 3.358 | 3.664 | 13.909 | 23.797 | 19.193 |
| Vss_obs | mL/kg | 277.881 | 258.387 | 257.986 | 172.910 | 182.410 | 180.716 | 219.652 | 156.723 | 200.561 |

What is claimed is:

1. An anti-IL-36R antibody or antigen-binding fragment thereof, comprising heavy chain complementary determining region 1 (HCDR1), HCDR2 and HCDR3, wherein
the HCDR1 comprises an amino acid sequence of DYYX₁X₂ (SEQ ID NO: 191), SEQ ID NO: 19, 33, 48, 64, 79, 94, 109, 124 or 139;
the HCDR2 comprises an amino acid sequence of LIRNKAAGYTIYYX₃X₄ X₅VKG (SEQ ID NO: 192), SEQ ID NO: 20, 34, 49, 65, 80, 95, 110, 125 or 140; and
the HCDR3 comprises an amino acid sequence of SEQ ID NO: 5, 21, 35, 50, 66, 81, 96, 111, 126 or 141;
wherein, X₁ is M or L; X₂ is N, H, S or R; X₃ is S or A; X₄ is A or D; X₅ is S or P.

2. An anti-IL-36R antibody or antigen-binding fragment thereof, comprising light chain complementary determining region 1 (LCDR1), LCDR2 and LCDR3, wherein
the LCDR1 comprises an amino acid sequence of RASX₁₈NINIWLS (SEQ ID NO: 193), SEQ ID NO: 22, 36, 51, 67, 82, 97, 112, 127 or 142;
the LCDR2 comprises an amino acid sequence of SEQ ID NO: 7, 23, 37, 52, 68, 83, 98, 113, 128 or 113; and
the LCDR3 comprises an amino acid sequence of X₁₉QSQSYPLT (SEQ ID NO: 194), SEQ ID NO: 24, 38, 53, 69, 84, 99, 114, 129 or 143;
wherein, X₁₈ is Q or R; X₁₉ is Q or L.

3. An anti-IL-36R antibody or antigen-binding fragment thereof, comprising heavy chain complementary determining region 1 (HCDR1), HCDR2 and HCDR3, and/or light chain complementary determining region 1 (LCDR1), LCDR2 and LCDR3, wherein
(a) the HCDR1 comprises an amino acid sequence of DYYX₁X₂ (SEQ ID NO: 191),
the HCDR2 comprises an amino acid sequence of LIRNKAAGYTIYY X₃X₄X₅VKG (SEQ ID NO: 192),
the HCDR3 comprises an amino acid sequence of SEQ ID NO: 5,
the LCDR1 comprises an amino acid sequence of RASX₁₈NINIWLS (SEQ ID NO: 193),
the LCDR2 comprises an amino acid sequence of SEQ ID NO: 7, and
the LCDR3 comprises an amino acid sequence of X₁₉QSQSYPLT (SEQ ID NO: 194),
wherein, X₁ is M or L; X₂ is N, H, S or R; X₃ is S or A; X₄ is A or D; X₅ is S or P; X₁₈ is Q or R; X₁₉ is Q or L;
(b) the HCDR1 comprises an amino acid sequence of SEQ ID NO: 19,
the HCDR2 comprises an amino acid sequence of SEQ ID NO: 20,
the HCDR3 comprises an amino acid sequence of SEQ ID NO: 21,
the LCDR1 comprises an amino acid sequence of SEQ ID NO: 22,
the LCDR2 comprises an amino acid sequence of SEQ ID NO: 23, and
the LCDR3 comprises an amino acid sequence of SEQ ID NO: 24;
(c) the HCDR1 comprises an amino acid sequence of SEQ ID NO: 33,
the HCDR2 comprises an amino acid sequence of SEQ ID NO: 34,
the HCDR3 comprises an amino acid sequence of SEQ ID NO: 35,
the LCDR1 comprises an amino acid sequence of SEQ ID NO: 36,
the LCDR2 comprises an amino acid sequence of SEQ ID NO: 37, and
the LCDR3 comprises an amino acid sequence of SEQ ID NO: 38;
(d) the HCDR1 comprises an amino acid sequence of SEQ ID NO: 48,
the HCDR2 comprises an amino acid sequence of SEQ ID NO: 49,
the HCDR3 comprises an amino acid sequence of SEQ ID NO: 50,
the LCDR1 comprises an amino acid sequence of SEQ ID NO: 51,
the LCDR2 comprises an amino acid sequence of SEQ ID NO: 52, and
the LCDR3 comprises an amino acid sequence of SEQ ID NO: 53;
(e) the HCDR1 comprises an amino acid sequence of SEQ ID NO: 64,
the HCDR2 comprises an amino acid sequence of SEQ ID NO: 65,
the HCDR3 comprises an amino acid sequence of SEQ ID NO: 66,
the LCDR1 comprises an amino acid sequence of SEQ ID NO: 67,
the LCDR2 comprises an amino acid sequence of SEQ ID NO: 68, and
the LCDR3 comprises an amino acid sequence of SEQ ID NO: 69;
(f) the HCDR1 comprises an amino acid sequence of SEQ ID NO: 79,
the HCDR2 comprises an amino acid sequence of SEQ ID NO: 80,
the HCDR3 comprises an amino acid sequence of SEQ ID NO: 81,
the LCDR1 comprises an amino acid sequence of SEQ ID NO: 82,
the LCDR2 comprises an amino acid sequence of SEQ ID NO: 83, and
the LCDR3 comprises an amino acid sequence of SEQ ID NO: 84;
(g) the HCDR1 comprises an amino acid sequence of SEQ ID NO: 94,
the HCDR2 comprises an amino acid sequence of SEQ ID NO: 95,
the HCDR3 comprises an amino acid sequence of SEQ ID NO: 96,
the LCDR1 comprises an amino acid sequence of SEQ ID NO: 97,
the LCDR2 comprises an amino acid sequence of SEQ ID NO: 98, and
the LCDR3 comprises an amino acid sequence of SEQ ID NO: 99;
(h) the HCDR1 comprises an amino acid sequence of SEQ ID NO: 109,
the HCDR2 comprises an amino acid sequence of SEQ ID NO: 110,
the HCDR3 comprises an amino acid sequence of SEQ ID NO: 111,
the LCDR1 comprises an amino acid sequence of SEQ ID NO: 112,
the LCDR2 comprises an amino acid sequence of SEQ ID NO: 113, and
the LCDR3 comprises an amino acid sequence of SEQ ID NO: 114;
(i) the HCDR1 comprises an amino acid sequence of SEQ ID NO: 124,
the HCDR2 comprises an amino acid sequence of SEQ ID NO: 125,
the HCDR3 comprises an amino acid sequence of SEQ ID NO: 126,
the LCDR1 comprises an amino acid sequence of SEQ ID NO: 127,
the LCDR2 comprises an amino acid sequence of SEQ ID NO: 128, and
the LCDR3 comprises an amino acid sequence of SEQ ID NO: 129; or
(j) the HCDR1 comprises an amino acid sequence of SEQ ID NO: 139,
the HCDR2 comprises an amino acid sequence of SEQ ID NO: 140,
the HCDR3 comprises an amino acid sequence of SEQ ID NO: 141,
the LCDR1 comprises an amino acid sequence of SEQ ID NO: 142,
the LCDR2 comprises an amino acid sequence of SEQ ID NO: 113, and
the LCDR3 comprises an amino acid sequence of SEQ ID NO: 143.

4. The antibody or antigen-binding fragment thereof of claim 1, wherein
the HCDR1 comprises an amino acid sequence of SEQ ID NO: 3, 180, 184 or 188,
the HCDR2 comprises an amino acid sequence of SEQ ID NO: 4, 151, 164 or 173,
the HCDR3 comprises an amino acid sequence of SEQ ID NO: 5,
the LCDR1 comprises an amino acid sequence of SEQ ID NO: 6 or 152,
the LCDR2 comprises an amino acid sequence of SEQ ID NO: 7, and
the LCDR3 comprises an amino acid sequence of SEQ ID NO: 8 or 153.

5. The antibody or antigen-binding fragment thereof of claim 1, wherein
(a) the HCDR1 comprises an amino acid sequence of SEQ ID NO: 3; the HCDR2 comprises an amino acid sequence of SEQ ID NO: 4; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 8;
(b) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153;
(c) the HCDR1 comprises an amino acid sequence of SEQ ID NO: 3; the HCDR2 comprises an amino acid sequence of SEQ ID NO: 164; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 153;
(d) the HCDR1 comprises an amino acid sequence of SEQ ID NO: 3; the HCDR2 comprises an amino acid sequence of SEQ ID NO: 173; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 153;
(e) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 6; the LCDR2 comprises an amino acid sequence of SEQ ID NO: 7; and the LCDR3 comprises an amino acid sequence of SEQ ID NO: 153;
(f) the HCDR1 comprises an amino acid sequence of SEQ ID NO: 180; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153;
(g) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153;
(h) the HCDR1 comprises an amino acid sequence of SEQ ID NO: 184; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153;
(i) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153;
(j) the HCDR1 comprises an amino acid sequence of SEQ ID NO: 188; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153; or
(k) the HCDR1 comprises an amino acid sequence of SEQ ID NO: 3; the HCDR2 comprises an amino acid sequence of SEQ ID NO: 151; the HCDR3 comprises an amino acid sequence of SEQ ID NO: 5; the LCDR1 comprises an amino acid sequence of SEQ ID NO: 152; the LCDR2 comprises an amino acid sequence of SEQ ID NO: 7; and the LCDR3 comprises an amino acid sequence of SEQ ID NO: 153.

6. The antibody or antigen-binding fragment thereof of claim 1, further comprising one or more of heavy chain framework region 1 (HFR1), HFR2, HFR3 and HFR4, and/or one or more of light chain framework region 1 (LFR1), LFR2, LFR3 and LFR4, wherein
(a) the HFR1 comprises an amino acid sequence of X₆VQLX₇ESGGGLV KPGGSLRLSCAASGX₈X₉FX₁₀ (SEQ ID NO: 195), or a homologous sequence of at least 85% sequence identity thereof,
the HFR2 comprises an amino acid sequence of WX₁₁RQAPGKGLEW VX₁₂ (SEQ ID NO: 196), or a homologous sequence of at least 85% sequence identity thereof,
the HFR3 comprises an amino acid sequence of RFTISRDX₁₃X₁₄KSX₁₅ LYLQMNSLX₁₆X₁₇EDTAVYYCVR (SEQ ID NO: 197), or a homologous sequence of at least 85% sequence identity thereof,
the HFR4 comprises an amino acid sequence of SEQ ID NO: 157, or
a homologous sequence of at least 85% sequence identity thereof,
the LFR1 comprises an amino acid sequence of X₂₀IVMTQSPX₂₁X₂₂X₂₃ SX₂₄SX₂₅GX₂₆RX₂₇TX₂₈X₂₉C (SEQ ID NO: 198), or a homologous sequence of at least 85% sequence identity thereof,
the LFR2 comprises an amino acid sequence of WYQQKPGX₃₀APX₃₁L FIY (SEQ ID NO: 199), or a homologous sequence of at least 85% sequence identity thereof,
the LFR3 comprises an amino acid sequence of GVPX₃₂RFSGSGSGTX₃₃FTLTISSLQX₃₄EDFAX₃₅YYC (SEQ ID NO: 200), or a homologous sequence of at least 85% sequence identity thereof, and
the LFR4 comprises an amino acid sequence of SEQ ID NO: 161, or a homologous sequence of at least 85% sequence identity thereof,
wherein, X₆ is Q or E; X₇ is Q or V; X₈ is F or Y; X₉ is A, D or N; X₁₀ is T or G; X₁₁ is I or V; X₁₂ is S or A; X₁₃ is N or D; X₁₄ is A or S; X₁₅ is S or T; X₁₆ is R or K; X₁₇ is A or T; X₂₀ is D or E; X₂₁ is S or A; X₂₂ is S or T; X₂₃ is L or V; X₂₄ is A or V; X₂₅ is V or P; X₂₆ is D or E; X₂₇ is V or A; X₂₈ is I or L; X₂₉ is T or S, X₃₀ is Q or K; X₃₁ is K or R; X₃₂ is S or A; X₃₃ is D or E; X₃₄ is S or P; X₃₅ is T or V.

7. The antibody or antigen-binding fragment thereof of any of the preceding claims, comprising a heavy chain variable region (VH) comprising an amino acid sequence selected from the group consisting of SEQ Id NOs: 1, 17, 31, 46, 62, 77, 92, 107, 122, 137, 149, 162, 171, 176, 179, 182, 183, 185, 187, and 189, or a homologous sequence thereof having at least 80% sequence identity.

8. The antibody or antigen-binding fragment thereof of any of the preceding claims, comprising a light chain variable region (VL) comprising an amino acid sequence selected from the group consisting of SEQ Id NOs: 2, 18, 32, 47, 63, 78, 93, 108, 123, 138, 150, 163, 172, and 177, or a homologous sequence thereof having at least 80% sequence identity.

9. The antibody or antigen-binding fragment thereof of any of the preceding claims, comprising a heavy chain variable region (VH) comprising an amino acid sequence selected from the group consisting of SEQ Id NOs: 1, 149, 162, 171, 176, 179, 182, 183, 185, 187, and 189, or a homologous sequence thereof having at least 80% sequence identity.

10. The antibody or antigen-binding fragment thereof of any of the preceding claims, comprising a light chain variable region (VL) comprising an amino acid sequence selected from the group consisting of SEQ Id NOs: 2, 150, 163, 172, and 177, or a homologous sequence thereof.

11. The antibody or antigen-binding fragment thereof of any of the preceding claims, further comprising one or more amino acid residue substitutions or modifications yet retains binding specificity to human IL-36R.

12. The antibody or an antigen-binding fragment thereof of claim 11, wherein at least one of the substitutions or modifications is in one or more of the complementarity determining region (CDR) sequences of the heavy chain variable region or light chain variable region.

13. The antibody or an antigen-binding fragment thereof of claim 11, wherein at least one of the substitutions or modifications is in one or more of the non-CDR sequences of the heavy chain variable region or light chain variable region.

14. The antibody or an antigen-binding fragment thereof of any one of the preceding claims, further comprising an Fc region, optionally an Fc region of human immunoglobulin (Ig), or optionally an Fc region of human IgG.

15. The antibody or an antigen-binding fragment thereof of claim 14, wherein the Fc region is derived from human IgG4.

16. The antibody or an antigen-binding fragment thereof of claim 15, wherein the Fc region derived from human IgG4 comprises mutations of M252Y/S254T/T256 (YTE), or T307Q/N434A (QA).

17. The antibody or an antigen-binding fragment thereof of claim 15, comprising a heavy chain constant region comprising an amino acid sequence selected from the group consisting of SEQ Id NOs: 201, 203 and 204.

18. The antibody or an antigen-binding fragment thereof of claim 16, comprising a light chain constant region comprising an amino acid sequence of SEQ ID NO: 202.

19. The antibody or an antigen-binding fragment thereof of any one of the preceding claims, which is humanized.

20. The antibody or an antigen-binding fragment thereof of any one of the preceding claims, which is a monoclonal antibody, a bispecific antibody, a multi-specific antibody, a recombinant antibody, a chimeric antibody, a labeled antibody, a bivalent antibody, an anti-idiotypic antibody or a fusion protein.

21. The antibody or an antigen-binding fragment thereof of any one of the preceding claims, which is a diabody, a Fab, a Fab′, a F(ab′)2, a Fd, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

22. The antibody or an antigen-binding fragment thereof of any one of the preceding claims, which does not bind to cynomolgus IL-36R or mouse IL-36R.

23. The antibody or an antigen-binding fragment thereof of any one of the preceding claims, which does not bind to human IL-1R1.

24. The antibody or an antigen-binding fragment thereof of any one of the preceding claims, having one or more binding properties to human IL-36R selected from the group consisting of:
a) having a binding affinity to recombinant human IL-36R protein with a K_{d} of no more than 2E-08 M (preferably no more than 1E-08 M, for example, no more than 9E-09M, 8E-09M, 7E-09M, 6E-09M, 5E-09M, 4E-09M, 3E-09M, 2E-09M, or 1E-10 M) as measured by Biolayer interferometry (BLI);
b) having a binding affinity to recombinant human IL-36R protein with a K_{d} of no more than 1E-08 M (for example, no more than 9E-09M, 8E-09M, 7E-09M, 6E-09M, 5E-09M, 4E-09M, 3E-09M, 2E-09M, or 1E-10 M;) as measured by Surface Plasmon Resonance (SPR);
c) having a binding affinity to recombinant human IL-36R protein with an EC₅₀ of no more than 0.1 μg/mL (for example, 0.09 μg/mL, 0.08 μg/mL, 0.07 μg/mL, 0.06 g/mL, 0.05 μg/mL, 0.04 μg/mL, 0.03 μg/mL, 0.02 μg/mL, 0.01 μg/mL, or 0.005 g/mL) as measured by enzyme linked immunosorbent assay (ELISA); or
d) having a binding affinity to membrane human IL-36R with an EC₅₀ of no more than 0.5 μg/mL (for example, 0.4 μg/mL, 0.3 μg/mL, 0.2 μg/mL, 0.19 μg/mL, 0.18 μg/mL, 0.17 μg/mL, 0.16 μg/mL, 0.15 μg/mL, or 0.1 μg/mL) as measured by Fluorescence activated Cell Sorting (FACS).

25. The antibody or antigen-binding fragment thereof of any one of the preceding claims, capable of blocking IL-36R signaling induced by an IL-36R agonist (preferably IL-36α), as measured by IL-36R reporter assay.

26. The antibody or antigen-binding fragment thereof of any one of the preceding claims, capable of inhibiting IL-36R agonist-induced IL-8 release in a cell, wherein the IL-36R agonist includes IL-36α, IL-36β, and/or IL-36γ.

27. The antibody or antigen-binding fragment thereof of any one of the preceding claims, having the ability of inhibiting IL-36R agonist-induced IL-6 release in a cell, wherein the IL-36R agonist includes IL-36α, IL-36β, and/or IL-36γ.

28. The antibody or antigen-binding fragment thereof of any one of the preceding claims, having the ability of inhibiting IL-36R agonist-induced TNF-α release in a cell, wherein the IL-36R agonist includes IL-36α, IL-36β, and/or IL-36γ.

29. The antibody or an antigen-binding fragment thereof of any one of the preceding claims, which is linked to one or more conjugate moieties.

30. The antibody or an antigen-binding fragment thereof of claim 29, wherein the conjugate moiety comprises an agent for detection or isolation, such as clearance-modifying agent, a luminescent label, a fluorescent label, an enzyme-substrate label, or a purification moiety.

31. An isolated polynucleotide encoding the antibody or an antigen-binding fragment thereof of any one of claims 1-30.

32. A vector comprising the isolated polynucleotide of claim 31.

33. A host cell comprising the vector of claim 32.

34. A pharmaceutical composition, comprising:
(i) the antibody or an antigen-binding fragment thereof of any one of claims 1-30, or the polynucleotide encoding the antibody or antigen-binding fragment thereof of any one of claims 1-30, or the recombinant immune cell of claim 35 or 36; and
(ii) one or more pharmaceutically acceptable carriers.

35. The pharmaceutical composition of claim 34, further comprising an additional therapeutic agent.

36. The pharmaceutical composition of claim 35, wherein the additional therapeutic agent is an agent for treating a disease or disorder that is responsive to IL-36R inhibition.

37. A method of expressing the antibody or an antigen-binding fragment thereof of any one of claims 1-30, comprising culturing the host cell of claim 33 under the condition at which the vector of claim 32 is expressed.

38. A method of treating, preventing or alleviating a disease or disorder that is responsive to IL-36R inhibition in a subject, comprising administering to the subject a therapeutically effective amount of the antibody or an antigen-binding fragment thereof of any one of claims 1-30, or the polynucleotide encoding the antibody or antigen-binding fragment thereof of any one of claims 1-30, and/or the pharmaceutical composition of any one of claim 34-36.

39. The method of claim 38, wherein the disease or disorder is associated with dysregulation of IL-36R mediated signaling.

40. The method of claim 39, wherein the dysregulation of IL-36R mediated signaling includes dysregulation of IL-36 (IL-36α, IL-36β, or IL-36γ), IL-36R antagonist and/or IL-38.

41. The method of claim 39 or 40, wherein the dysregulation of IL-36R mediated signaling includes over activation of IL-36 (IL-36α, IL-36β, or IL-36γ), or over suppression of IL-36R antagonist and IL-38, compared with control level.

42. The method of claim 41, wherein the control level is the level in a healthy subject.

43. The method of claim 38, wherein the disease or disorder is selected from the group consisting of: inflammatory disease, an autoimmune disease, a respiratory disease, a metabolic disorder, and cancer.

44. The method of claim 43, wherein the inflammatory disease is selected from the group consisting of: allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, and other allergies.

45. The method of claim 44, wherein the inflammatory disease is selected from the group consisting of: allergic inflammation of the skin, lungs, and gastrointestinal tract, atopic dermatitis (also known as atopic eczema), asthma (allergic and non-allergic), and epithelial-mediated inflammation.

46. The method of claim 43, wherein the autoimmune disease is selected from the group consisting of: multiple sclerosis, asthma, type 1 diabetes mellitus, rheumatoid arthritis, scleroderma, Crohn's disease, psoriasis vulgaris (commonly referred to as psoriasis), hidradenitis suppurativa, generalized pustular psoriasis (GPP), palmo-plantar pustulosis (PPP), inflammatory bowel disease, psoriatic arthritis, systemic lupus erythematosus (SLE), ulcerative colitis, ankylosing spondylitis, atopic dermatitis, acne vulgaris, and Behcet's disease.

47. The method of claim 46, wherein the autoimmune disease is selected from the group consisting of: psoriasis vulgaris (commonly referred to as psoriasis), hidradenitis suppurativa, generalized pustular psoriasis (GPP), palmo-plantar pustulosis (PPP), inflammatory bowel disease, atopic dermatitis, acne vulgaris, and Behcet's disease.

48. The method of claim 43, wherein the respiratory disease is selected from the group consisting of: asthma, cystic fibrosis, emphysema, chronic obstructive pulmonary disease (COPD), and acute respiratory distress syndrome.

49. The method of claim 43, wherein the metabolic disease is selected from the group consisting of: obesity, type 2 diabetes, atherosclerosis, and cardiovascular disease.

50. The method of claim 43, wherein the cancer can be any type of cancer known in the art, including but not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, leukemia, lymphoma, and Merkel cell carcinoma.

51. The method of claim 38, wherein the disease or disorder is selected from the group consisting of: psoriasis, pustular psoriasis, hidradenitis suppurativa, ichthyosis, inflammatory bowel disease (IBD), atopic dermatitis, acne vulgaris, and Behcet's disease.

52. The method of any one of claims 38-51, wherein the subject is human.

53. The method of any one of claims 38-52, wherein the administration is via oral, nasal, intravenous, subcutaneous, sublingual, or intramuscular administration.

54. A method of detecting the presence or amount of IL-36R in a sample, comprising contacting the sample with the antibody or an antigen-binding fragment thereof of any one of claims 1-30, and determining the presence or the amount of IL-36R in the sample.

55. The method of claim 54, further comprising a step of determining whether IL-36R is over-expressed in the cells in the sample.

56. Use of the antibody or an antigen-binding fragment thereof of any one of claims 1-30, and/or the pharmaceutical composition of any one of claims 34-36 in the manufacture of a medicament for treating, preventing or alleviating a disease or disorder that is responsive to IL-36R inhibition.

57. A chimeric antigen receptor (CAR) comprising an antigen binding domain, a transmembrane domain, and a TCR signaling domain, wherein the antigen binding domain specifically binds to IL-36R and comprises an antigen binding fragment of any of claims 1-30.

58. The CAR of claim 57, wherein the CAR further comprises a costimulatory domain.

59. A nucleic acid sequence encoding the chimeric antigen receptor (CAR) of claim 57 or 58.

60. A cell comprising the nucleic acid sequence of claim 59.

61. The cell genetically modified to express the CAR of claim 57 or 58.

62. A vector comprising the nucleic acid sequence of claim 59.

63. A method for stimulating a T cell-mediated immune response to an IL-36R-expressing cell or tissue in a mammal, the method comprising administering to the mammal an effective amount of a cell genetically modified to express the CAR of claim 57 or 58.

64. A method of treating a mammal having an IL-36R related disease or condition, comprising administering to the mammal an effective amount of a cell of claim 61, thereby treating the mammal.

65. The method of claim 64, wherein the cell is an autologous T cell.

66. The method of claim 64, wherein the mammal is a human subject.

67. The method claim 64, wherein the mammal is identified as having an IL-36R positive cell, or a cell with IL-36R signaling up-regulated.
